(19) United States
(12) Patent Application Publication
(10) Pub. No.: US 2015/0305974 A1
(43) Pub. Date: Oct. 29, 2015

(54) METHODS AND DEVICES FOR TREATING HYPERTENSION

(71) Applicant: Sympara Medical, Inc., San Francisco, CA (US)

(72) Inventors: Kevin Joe Ehrenreich, San Francisco, CA (US); Randolf von Oepen, Aptos, CA (US); William Rehlich, San Francisco, CA (US); Kelly Justin McCrystle, Menlo Park, CA (US)

(21) Appl. No.: 14/694,767

(22) Filed: Apr. 23, 2015

Related U.S. Application Data
(60) Provisional application No. 61/983,682, filed on Apr. 24, 2014.
(60) Provisional application No. 62/014,644, filed on Jun. 19, 2014.
(60) Provisional application No. 62/023,730, filed on Jul. 11, 2014.

(51) Int. Cl. A61H 23/02; A61B 5/00; A61H 23/04; A61B 5/022

(52) U.S. Cl. CPC A61H 23/0236; A61B 5/02208; A61B 5/6833; A61H 23/0245; A61H 23/04; A61B 5/6843; A61B 2562/0219; A61H 2230/045; A61H 2230/065; A61H 2230/305

(57) ABSTRACT

Devices, systems and methods are described which control blood pressure and nervous system activity by stimulating baroreceptors. By selectively and controllably activating baroreceptors and/or nerves, the present disclosure, according to some embodiments reduces blood pressure and alters the sympathetic nervous system; thereby minimizing deleterious effects on the heart, vasculature and other organs and tissues. A baroreceptor activation device or other sensory activation device is positioned near a dermal bone to provide the treatment.

### INCORPORATION BY REFERENCE TO RELATED APPLICATIONS

This application claims the benefit under 35 U.S.C. §119(e) as a nonprovisional application of U.S. Provisional Application No. 61/983,682, filed Apr. 24, 2014, U.S. Provisional Application No. 62/014,644, filed Jun. 19, 2014, and U.S. Provisional Application No. 62/023,730, filed Jul. 11, 2014. The disclosure of each of the aforementioned applications in this paragraph is incorporated by reference herein in its entirety.

### BACKGROUND

1. Field of the Invention

This disclosure relates generally in some aspects to methods and devices for the treatment of hypertension. More specifically, methods and devices which treat hypertension using devices disposed extra corporally.

2. Description of the Related Art

Hypertension, or high blood pressure, affects millions of people every day and is a serious health hazard. Hypertension is associated with an elevated risk for heart attack, heart failure, arterial aneurysms, kidney failure and stroke. There are many factors that may affect blood pressure, such as: salt intake, obesity, occupation, alcohol intake, smoking, pregnancy, stimulant intake, sleep apnea, genetic susceptibility, decreased kidney perfusion, arterial hardening and medication(s). Many times people are unaware that they suffer from hypertension until it is discovered during a medical check-up with their health care practitioner (HCP), or worse, it is discovered when they are hospitalized for a hypertension related condition such as a heart attack or stroke.

Blood pressure is controlled by a complex system within the body, one component of this system is known as the arterial baroreflex (ABR). The baroreflex is the fastest autonomic reflex responding to changes in blood pressure. The baroreceptor nerve endings are embedded in vessels throughout the circulatory system and encode both mean pressure and rate of change of pressure as a frequency. Centers in the brainstem process spikes in the frequency information, integrating it with other information and providing a signal to the sinoatrial (SA) pacemaking node of the heart via efferent fibers in the vagus nerve. When blood pressure becomes too high, the resulting vagal nerve signal triggers the release of acetylcholine at the SA node of the heart, slowing the heart rate and thus lowering the blood pressure.

Concentrations of baroreceptors are located in the transverse aortic arch and the carotid sinuses of the left and right internal carotid arteries. The baroreceptors found within the aortic arch monitor the pressure of blood delivered to the systemic circuit, and the baroreceptors within the carotid arteries monitor the pressure of the blood being delivered to the brain.

As described above, the arterial baroreceptors are stretch receptors that are stimulated by distortion of the arterial wall when pressure changes. The baroreceptors can identify the changes in the average blood pressure or the rate of change in pressure with each arterial pulse. Action potentials triggered in the baroreceptor endings are then conducted to the brainstem where central terminations (synapses) transmit this information to neurons within the solitary nucleus. Reflex responses from such baroreceptor activity can trigger increases or decreases in the heart rate. Arterial baroreceptor (ABR) sensory endings are simple, splayed nerve endings that lie in the tunica adventitia of the artery. An increase in the mean arterial pressure increases depolarization of these sensory endings, which results in action potentials. These action potentials are conducted to the solitary nucleus in the central nervous system by axons and have a reflex effect on the cardiovascular system through autonomic neurons.

At normal resting blood pressures, baroreceptors discharge at approximately 1 out of every 3 heart beats. If blood pressure falls, the arteries retract in diameter and the baroreceptor firing rate decreases with the drop in blood pressure and the brain sends a signal to the heart to increase blood pressure by increasing heart rate. The brain also sends a signal to dilate the arteries and veins of the periphery. Signals from the carotid baroreceptors are sent via the glossopharyngeal nerve (cranial nerve IX). Signals from the aortic baroreceptors travel through the vagus nerve (cranial nerve X). Arterial baroreceptors inform reflexes about arterial blood pressure.

The arterial baroreflex system is a dynamic system that is capable of adapting to ever changing situations. The ABR is the reason why we do not pass out when moving from a seated to standing position. In this instance the ABR senses a change in blood pressure and accommodates the change by sending the appropriate signal to regulate blood pressure. The ABR system also performs an essential function to regulate blood pressure during exercise, wherein during exercise your heart rate increases as well as your blood pressure, however, at a certain point during exercise the ABR will intervene, allowing the heart rate to further increase but not allowing the blood pressure to further increase.

As stated above, hypertension currently affects a large and growing population. Currently treatments for hypertension range from prescribed lifestyle changes and the use of pharmaceutical products. Within the past couple of years, new surgical therapies are emerging. These surgical therapies either lead to the implantation of a device for stimulating a patient's carotid baroreceptor or to the disconnection of the nerves of the renal arteries.

If prescribed lifestyle changes do not address a patient's hypertension, their HCP will typically prescribe drug therapy to treat their hypertension. There are multiple classes of pharmaceutical products that can be utilized to treat hypertension. These include vasodilators to reduce the blood pressure and ease the workload of the heart, diuretics to reduce fluid overload, inhibitors and blocking agents of the body's neurohormonal responses, and other medicaments or medications. Many times, a HCP will prescribe one or more of these products to a patient to be taken in combination in order to lower their blood pressure. However, the use of pharmaceutical products is not without their risks. Many of these products carry warnings of potential side effects. Additionally, each patient may respond differently to the products, therefore multiple office visits may be required before the right dosage and type of pharmaceutical products are selected, which leads to greater health care costs. Further still there are a number of patients who either do not respond to medication, refuse to take medication, or over time the medication no longer provides a therapeutic effect. Recently, new clinical trial data has drawn correlations between the use of diuretic pharmaceutical products to treat high blood pressure and the incident or occurrence of diabetes within the patient.

For patients who do not respond to drug therapy, there are medical devices and treatments that can be utilized to treat high blood pressure. Some of these devices involve invasive surgical procedures including the implantation of a permanent medical device within a patient's artery to impart a force at a specific location within the artery which then may cause a lowering of blood pressure. However, these devices are relatively new or are still under development and have not been proven over a long period of time. Also, since the device is a permanent implant, there is always the possibility of complications during the implantation process or infections related to the implantation.

As described above, another type of invasive medical device is an electrical signal generating implant, where electrodes are placed adjacent to the carotid artery. With this process, the surgeon must be careful not to sever any of the nerves while implanting the device. If the nerves are severed, then the device will not function properly and may lead to long term health complications for the patient. However, even more troubling is that the patient has now permanently lost one of the body's key concentrations of baroreceptors for controlling blood pressure naturally, which may lead to complications later, which are currently unknown. Additionally, the implant device requires regular battery replacement, which to do so requires another invasive surgical procedure and also increased cost to the medical system and user.

Another type of invasive medical device and procedure being developed is the use of ablation catheter to denervate the carotid body, specifically the chemoreceptors of the carotid body. Similar to the device and procedure described above, this device permanently causes a disconnection between the chemoreceptors and the nervous system/brain. The long term effects are unknown, additionally, other nerves may be destroyed or disconnected during the procedure which may lead to other side effects.

Another type of invasive medical procedure to treat hypertension being developed is to use an ablation catheter placed within the renal artery, where a series of energy pulses are performed to ablate (sever) the nerves surrounding the artery, thereby effectively disconnecting the nerves of the kidney from the body. This procedure results in a permanent and non-reversible change to the patient's nervous system, this procedure is being referred to as renal nerve ablation or renal denervation. The long term effects of such a permanent treatment are unknown at this time as this approach is relatively new on the market. Recently published data has shown that not all patients respond to this surgical procedure, that is after the procedure, some of the patients show little to no changes in their blood pressure. This may be concerning as now these patients have had their renal arteries permanently disconnected from the nervous system leading to their kidneys, which may lead to long term effects which are unknown at this time. Additionally, the costs associated with an invasive medical procedure are not insignificant, only to prove that the procedure had no effect, thus, instead of potentially lowering the cost of treatment for these patients, the cost of treating their hypertension was significantly added to.

Additionally, the recently published data also shows that patients who respond to renal denervation may still remain hypertensive. Thus, the renal denervation procedure may not be a “cure,” instead it may be seen as an adjunctive therapy, as such these patients may remain on drug therapies or are recommended to remain on drug therapy after having undergone renal denervation.

Yet another invasive surgical approach to address hypertension is a combination of a device and a pharmaceutical product, wherein a catheter with a needle disposed near its distal end are placed within the renal artery. Once in position, a liquid pharmaceutical product is injected into the wall of the artery or into the area surrounding the wall of the artery, whereby the pharmaceutical product is designed to chemically ablate the renal nerves. Here again, this treatment procedure is considered to be a permanent alteration of the nerve traffic between the brain and kidney, whereby the nerves are permanently severed. Long term efficacy of the severing of the renal nerves is unknown. Additionally, long term effects of the procedure are also unknown.

Human skin acts as the protective barrier between our internal body systems and the outside world. Our skin in combination with our bodies nerves provides for the ability to perceive touch sensations and gives our brains a wealth of information about the environment around us, such as temperature, pain, and pressure. Without such a nervous system, we wouldn't be able to feel our feet hitting the floor when we walked, we wouldn't sense when something sharp cut us, and we wouldn't feel the warmth of the sun on our skin.

Human skin is composed of several layers. The very top layer is the epidermis and is the layer of skin you can see. In Latin, the prefix “epi-” means “upon” or “over,” thus the epidermis is the layer upon which the dermis is disposed (the dermis is the second layer of skin). The epidermis, made of dead skin cells, is waterproof and serves as a protective wrap for the underlying skin layers and the rest of the body. It contains melanin, which protects against the sun's harmful rays and also gives skin its color. When you are in the sun, the melanin builds up to increase its protective properties, which also causes the skin to darken. The epidermis also contains very sensitive cells called touch receptors that give the brain a variety of information about the environment the body is in.

The second layer of skin is the dermis. The dermis contains hair follicles, sweat glands, sebaceous (oil) glands, blood vessels, nerve endings, and a variety of touch receptors. The dermis' primary function is to sustain and support the epidermis by diffusing nutrients to it and replacing the skin cells that are shed off the upper layer of the epidermis. New cells are formed at the junction between the dermis and epidermis, and they slowly push their way towards the surface of the skin so that they can replace the dead skin cells that are shed. Oil and sweat glands eliminate waste produced at the dermis level of the skin by opening their pores at the surface of the epidermis and releasing the waste.

The bottom skin layer is the subcutaneous tissue which is composed of fat and connective tissue. The layer of fat acts as an insulator and helps regulate body temperature. It also acts as a cushion to protect underlying tissue from damage when you bump into things. The connective tissue keeps the skin attached to the muscles and tendons underneath.

Our sense of touch is controlled by a huge network of nerve endings and touch receptors disposed within the skin which is known as the somatosensory system. This system is responsible for all the sensations we feel; cold, hot, smooth, rough, pressure, tickle, itch, pain, vibrations, and more. Within the somatosensory system, there are four main types of receptors; mechanoreceptors, thermoreceptors, nociceptors, and proprioceptors.

It is important to understand how specialized receptors adapt to a change in stimulus (anything that touches the skin and causes sensations such as hot, cold, pressure, tickle, etc.). A touch receptor is considered rapidly adapting if it responds to a change in stimulus very quickly. This means that it can sense right away when the skin is touching an object and when it stops touching that object. However, rapidly adapting receptors can't sense the continuation and duration of a stimulus touching the skin (how long the skin is touching an object). These receptors best sense vibrations occurring on or within the skin. A touch receptor is considered slowly adapting if it does not respond to a change in stimulus very quickly. These receptors are very good at sensing the continuous pressure of an object touching or indenting the skin but are not very good at sensing when the stimulus started or ended.

Mechanoreceptors are receptors which perceive sensations such as pressure, vibrations, and texture. There are four known types of mechanoreceptors whose only function is to perceive indentions and vibrations of the skin: Merkel's disks, Meissner's corpuscles, Ruffini's corpuscles, and Pacinian corpuscles.

The most sensitive mechanoreceptors, Merkel's disks and Meissner's corpuscles, are found in the very top layers of the dermis and epidermis and are generally found in non-hairy skin such as the palms, lips, tongue, soles of feet, fingertips, eyelids, and the face. Merkel's disks are slowly adapting receptors and Meissner's corpuscles are rapidly adapting receptors so your skin can perceive both when you are touching something and how long the object is touching the skin.

Located deeper in the dermis and along joints, tendons, and muscles are Ruffini's corpuscles and Pacinian corpuscles. These mechanoreceptors can feel sensations such as vibrations traveling down bones and tendons, rotational movement of limbs, and the stretching of skin.

Another type of receptors are thermoreceptors, as their name suggests, these receptors perceive sensations related to the temperature of objects the skin feels. They are found in the dermis layer of the skin. There are two basic categories of thermoreceptors: hot and cold receptors.

Cold receptors start to perceive cold sensations when the surface of the skin drops below 95° F. They are most stimulated when the surface of the skin is at 77° F. and are no longer stimulated when the surface of the skin drops below 41° F. This is why your feet or hands start to go numb when they are submerged in icy water for a long period of time.

Hot receptors start to perceive hot sensations when the surface of the skin rises above 86° F. and are most stimulated at 113° F. But beyond 113° F., pain receptors take over to avoid damage being done to the skin and underlying tissues.

Thermoreceptors are found all over the body, but cold receptors are found in greater density than heat receptors. The highest concentration of thermoreceptors can be found in the face and ears.

Another type of receptor are pain receptors, commonly known as nociceptors, “Noci-” in Latin means “injurious” or “hurt.” These receptors detect pain or stimuli that can or does cause damage to the skin and other tissues of the body. There are over three million pain receptors throughout the body, found in skin, muscles, bones, blood vessels, and some organs. They can detect pain that is caused by mechanical stimuli (cut or scrape), thermal stimuli (burn), or chemical stimuli (poison from an insect sting).

These receptors cause a feeling of sharp pain to encourage you to quickly move away from a harmful stimulus such as a broken piece of glass or a hot stove stop. They also have receptors that cause a dull pain in an area that has been injured to encourage you not to use or touch that limb or body part until the damaged area has healed. While it is never a pleasant experience to activate these receptors that cause pain, these receptors play an important part in keeping the body safe from serious injury or damage by sending these early warning signals to the brain.

Another receptor type are proprioceptors, the word “proprius” means “one's own” and is used in the name of these receptors because they sense the position of the different parts of the body in relation to each other and the surrounding environment. Proprioceptors are found in tendons, muscles, and joint capsules. This location in the body allows these special cells to detect changes in muscle length and muscle tension. Without proprioceptors, we would not be able to do fundamental things such as feeding or clothing ourselves.

While many receptors have specific functions to help us perceive different touch sensations, almost never are just one type active at any one time. When drinking from a freshly opened can of soda, your hand can perceive many different sensations just by holding it. Thermoreceptors are sensing that the can is much colder than the surrounding air, while the mechanoreceptors in your fingers are feeling the smoothness of the can and the small fluttering sensations inside the can caused by the carbon dioxide bubbles rising to the surface of the soda. Mechanoreceptors located deeper in your hand can sense that your hand is stretching around the can, that pressure is being exerted to hold the can, and that your hand is grasping the can. Proprioceptors are also sensing the hand stretching as well as how the hand and fingers are holding the can in relation to each other and the rest of the body.

None of the sensations described above and felt by the somatosensory system would make any difference if these sensations could not reach the brain. The nervous system of the body takes up this important task. Neurons, which are specialized nerve cells that are the smallest unit of the nervous system, receive and transmit messages with other neurons so that messages can be sent to and from the brain. This allows the brain to communicate with the body. When your hand touches an object, the mechanoreceptors in the skin are activated, and they start a chain of events by signaling to the nearest neuron that they touched something. This neuron then transmits this message to the next neuron which gets passed on to the next neuron and on it goes until the message is sent to the brain. Now the brain can process what your hand touched and send messages back to your hand via this same pathway to let the hand know if the brain wants more information about the object it is touching or if the hand should stop touching it.

Vibration experiments have been conducted to test the effects of vibration, the results of such an experiment were published in 1961 in the Journal of Physiol. (1961), 159 pp 391-409, entitled “Response of Pacinian Corpuscles to Sinousoidal Vibration, by M. Sato. In this experiment it was proven that vibrations can excite the nervous system similar to utilization of electrical stimulation.

Other experiments have shown that the 1st Node of Ranvier gaps can be excited by either mechanical transduction or acoustic stimulation. The 1st Node of Ranvier gaps are gaps formed between myelin sheaths between different cells.

In a 1967 publication entitled “The Relative Sensitivity to Vibration of Muscle Receptors of the Cat,” M. C. Brown, I. Engberger and P. B. C. Matthews, Journal Physiol. (1967), 192 PP 773-800, the authors tested vibrations and concluded that vibratory effects persist as long as the vibration continues. Additionally, the authors cited another publication, 1966 Matthews, “Reflex excitation of the soleus muscle of the decerebrate cat caused by vibration applied to tendon” where vibration, was applied to a non-contracting muscle, provides a way of selectively activating nearly all of the nerve fibers from the primary endings to discharge repetitively. In contrast to electrical stimulation, vibration provides for a more likely activation.

Electrical stimulation will stimulate those nerves which are located in the close proximity to the electrical source, however, electrical stimulation will seek the lowest resistance pathway and is typically localized to the area of application. In contrast, vibrational stimulation carries the benefit of exciting afferent fibers at a distance from the location of the application of the vibration.

In 2000 a publication by Alfrey entitled “Characterizing the Afferent Limb of the Baroreflex” Rice University, Houston Tex., April 2000, UMI Microform 99-69-223. The author concluded that the baroreflex is the fastest autonomic reflex responding to changes in blood pressure. Baroreceptor nerve endings embedded in vessels throughout the circulatory system encode both mean pressure and rate of change of pressure as a frequency-modulated train of action potentials (spikes). Centers in the brainstem process the spike train information, integrating it with information from higher centers and providing a signal to the sinoatrial (SA) pacemaking node of the heart via efferent fibers in the vagus nerve. When blood pressure becomes too high, the resulting vagal signal triggers the release of acetylcholine at the SA node of the heart slowing heart rate and thus lowering blood pressure.

In another paper, published in 2004 by Syntichaki et al., entitled “Genetic Models of Mechanotransduction: The Nematode Caenorhabditis elegans” Physol Rev. 84: 1097-1153, 2004 10.1152/physrev.0043.2003, it was found that all vertebrates respond to similar mechanosensory stimuli, therefore it's likely that two humans would have similar response to the same wavelengths or frequencies.

It is further contemplated that the device and method(s) disclosed herein can be beneficial for other therapeutic uses such as for the treatment of sleep apnea and/or snoring.

Further still, in accordance with the present disclosure, according to some embodiments and treatment methods disclosed herein, a user or the therapy providing system described herein may additionally have a feeling of calmness or a soothing/warmth feeling during or after the use of the therapy providing device in accordance with the present disclosure, according to some embodiments.

Lastly, while there are number of different therapies available on the market and new therapies emerging, there are patient populations that cannot be treated through the use of the existing drugs or devices.

One such population is patients who develop high blood pressure during pregnancy. Health care practitioners are generally hesitant to prescribed pharmaceutical products in these situations as there may be unknown side effects to the mother and unborn child. Furthermore, many hypertensive pharmaceutical products have not been properly tested for use during pregnancy; therefore, there is much hesitancy on behalf of the prescribing physician to use such drug products due to potential untested side-effects as well as potential litigation arising from a side-effect. Pregnancy induced hypertension, gestational hypertension or preeclampsia may not be a permanent condition and may be resolve after delivery. Therefore, the use of permanent therapies, such as renal denervation, may not be warranted in this situation. Additionally, surgical procedures are not generally recommended during pregnancy.

There is yet another hypertensive population emerging in today's world is the hypertensive adolescent such as a child under 18 years of age. Over the past 30 years, the number of adolescent hypertensives has risen to a rate of over 3.7% diagnosed hypertensive and 3.4% diagnosed pre-hypertensive. Only 1 in 4 adolescents are currently diagnosed. Many of the currently available pharmaceutical products have not been tested on an adolescent population, therefore, as described above, many physicians are hesitant to prescribe drug therapies due to unknown side effects or long term effects they may have. Furthermore, the adolescent population poses yet another difficulty in that they are still developing and undergoing puberty and bone growth. Therefore, there is a need for a non-invasive, non-pharmaceutical solution to address this growing patient population.

Thus, it would be desirable to provide improved methods, devices and systems for artificial and selective activation of a patient's baroreflex or nervous system in order to achieve a variety of therapeutic objectives, including the control of hypertension, renal function, heart failure, and the treatment of other cardiovascular disorders. It would be particularly desirable if such methods and systems were non-invasive, reversible, safe and/or external to the patient.

### SUMMARY

In accordance with the present disclosure, according to some embodiments there is provided a device for treatment of hypertension, comprising, a housing, the housing have a proximal end and a distal end; and a driver assembly within the housing, the driver assembly electrically coupled to an energy source, the energy source disposed within the housing.

In accordance with the present disclosure, according to some embodiments there is provided a device for imparting energy to a patient, comprising, a housing, the housing having a proximal end, a distal end and defining a volume therebetween; a driver assembly is disposed within the volume of the housing; an energy source coupled to the driver assembly; and an electronics module coupled to the driver assembly and the energy source, wherein the electronics module controls the driver assembly.

In accordance with the present disclosure, according to some embodiments there is provided a device for treating hypertension, the device comprising, a housing, the housing having a proximal surface and a distal surface, wherein the housing further includes a mounting system, the mounting system including a first member and a second member, the first member associated with the housing and the second member configured to be received by tissue; and a driver assembly within the housing.

In accordance with the present disclosure, according to some embodiments this is provided a device for imparting energy to a patient, the device comprising: a housing, the housing having a first surface and a second surface, the surfaces defining a volume therebetween, wherein the housing further includes a mounting system, the mounting system including a first member and a second member, the first member associated with the housing and the second member configured to be received by tissue; a driver assembly disposed within the volume of the housing; an energy source coupled to the driver assembly; and an electronics module coupled to the driver assembly and the energy source.

In accordance with the present disclosure, according to some embodiments there is provided a method of providing therapy, the method comprising: applying a therapy applying device to a collar bone of a patient; and activating a driver assembly within the therapy applying device.

In accordance with the present disclosure, according to some embodiments, there is provided a device for modulating blood pressure comprising a vibration source configured to apply vibratory energy to the clavicle through the skin surface and a bandage. In some embodiments, the bandage comprises at least a first layer and a second layer and at least one of the layers includes an aperture formed therethrough and sized to receive a portion of the vibration source. In some embodiments, the bandage is adapted to adhesively attach to a skin surface over, for instance, a clavicle of a subject. In some embodiments, at least one of the layers of the bandage comprises at least one biologically active compound. In some embodiments, the biologically active compound is spaced apart from the vibration source forming a biologically compound-free area on the skin when the bandage is attached to the skin surface.

In accordance with the present disclosure, according to some embodiments there is also provided a device for modulating blood pressure comprising a vibration source configured to apply vibratory energy to the clavicle through the skin surface and a bandage. In some embodiments, the bandage comprises at least a first layer and a second layer and at least one of the layers includes an aperture formed therethrough and sized to receive a portion of the vibration source. In some embodiments, the bandage is adapted to adhesively attach to a skin surface over, for instance, a clavicle of a subject. In some embodiments, the first layer and second layer of the bandage comprise an adhesive forming a discontinuous layer comprising an adhesive-free zone.

In accordance with the present disclosure, according to some embodiments there is provided a method for modulating blood pressure, comprising positioning a device comprising a vibration source and at least one biologically-active compound on the skin surface of a patient; activating the vibration source such that the vibration source modulates the patient's baroreceptors, thereby decreasing the blood pressure of the patient; and absorbing the biologically-active compound transdermally through the skin surface of the patient.

### BRIEF DESCRIPTION OF THE DRAWINGS

FIG. 1 is an exemplary embodiment of the therapy system in accordance with the present disclosure;

FIG. 2 is an isometric view of a therapy providing device in accordance with the present disclosure, according to some embodiments;

FIGS. 3A-3D are exemplary illustrations of housings of the therapy providing device in accordance with the present disclosure, according to some embodiments;

FIG. 4A is a top view of a housing of the therapy providing device in accordance with the present disclosure, according to some embodiments;

FIG. 4B is a cross-sectional view of the housing of FIG. 4A taken about line B-B;

FIG. 4C is a cross-sectional view of another housing in accordance with the present disclosure, according to some embodiments;

FIG. 5A is an exploded view of a therapy providing device in accordance with the present disclosure, according to some embodiments;

FIGS. 5B and 5C illustrate an additional embodiment of a therapy providing device in accordance with the present disclosure, according to some embodiments;

FIG. 5D illustrates an additional embodiment of a housing in accordance with the present disclosure, according to some embodiments;

FIG. 5E illustrates an additional embodiment of a housing in accordance with the present disclosure, according to some embodiments;

FIG. 5F illustrates an additional embodiment of a housing in accordance with the present disclosure, according to some embodiments;

FIG. 5G illustrates an additional embodiment of a housing in accordance with the present disclosure, according to some embodiments;

FIG. 5H illustrates an additional embodiment of a housing in accordance with the present disclosure, according to some embodiments;

FIG. 5I illustrates an additional embodiment of a housing in accordance with the present disclosure, according to some embodiments;

FIG. 5J illustrates an additional embodiment of a housing in accordance with the present disclosure, according to some embodiments;

FIG. 5K illustrates an additional embodiment of a housing in accordance with the present disclosure, according to some embodiments;

FIG. 5L illustrates an additional embodiment of a housing in accordance with the present disclosure, according to some embodiments;

FIG. 5M illustrates an additional embodiment of a housing in accordance with the present disclosure, according to some embodiments;

FIG. 5N illustrates an additional embodiment of a housing in accordance with the present disclosure, according to some embodiments;

FIGS. 5P and 5Q illustrates an additional embodiment of a housing in accordance with the present disclosure, according to some embodiments;

FIGS. 5R and 5S illustrates an additional embodiment of a housing in accordance with the present disclosure, according to some embodiments;

FIGS. 5T and 5U illustrates an additional embodiment of a housing in accordance with the present disclosure, according to some embodiments;

FIG. 5V illustrates an additional embodiment of a housing in accordance with the present disclosure, according to some embodiments;

FIGS. 5W and 5X illustrates an additional embodiment of a housing in accordance with the present disclosure, according to some embodiments;

FIG. 5Y illustrates an exploded view of an additional embodiment of a therapy providing device in accordance with the present disclosure, according to some embodiments;

FIG. 5Z illustrates the flexible arm member of the embodiment of FIG. 5Y;

FIG. 5AA is a top view of a flexible arm in accordance with the present disclosure, according to some embodiments;

FIG. 5BB is a bottom view of the flexible arm of FIG. 5AA;

FIG. 5CC is a side view of the flexible arm of FIG. 5AA;

FIG. 6 is an isometric view of a circuit board in accordance with the present disclosure, according to some embodiments;

FIG. 7 is a plan view of a haptic speaker in accordance with the present disclosure, according to some embodiments;

FIG. 8 is an isometric view of an electroactive polymer transducer in accordance with the present disclosure, according to some embodiments;

FIG. 9 illustrates a cross-sectional view of the electroactive polymer transducer of FIG. 8 in communication with a driver;

FIG. 10 is a plan view of an alternative embodiment of an electroactive polymer transducer in accordance with the present disclosure, according to some embodiments;

FIG. 11 is an isometric view of a charging/base station in accordance with the present disclosure, according to some embodiments;

FIG. 12 is a side view of the therapy providing device of the present disclosure, according to some embodiments in combination with a CPAP mask assembly;

FIG. 13A is a bottom view illustrating a therapy device including an adhesive mounting system;

FIG. 13B illustrates the therapy device of FIG. 13A as disposed on a user;

FIGS. 14A and 14B illustrates and alternative mounting arrangement for the therapy device of the present disclosure, according to some embodiments;

FIG. 14C is a cross-sectional view of the mounting system of FIGS. 14A and 14B;

FIGS. 15A and 15B illustrate another mounting arrangement for the therapy device of the present disclosure, according to some embodiments;

FIGS. 16A and 16B illustrate a magnetic mounting system in accordance with the present disclosure, according to some embodiments;

FIGS. 16C and 16D illustrate a bandage mounting system in accordance with the disclosure;

FIG. 16E illustrates another bandage mounting system in accordance with the disclosure;

FIG. 16F illustrates a bandage in accordance with the present disclosure, according to some embodiments;

FIG. 16G illustrates a bandage in accordance with the present disclosure, according to some embodiments, illustrating conducting elements;

FIG. 16H illustrates an embodiment of a bandage having additional apertures formed therein;

FIG. 16I illustrates an embodiment of another bandage design in accordance with the disclosure;

FIG. 16J illustrates an embodiment of another bandage design in accordance with the disclosure;

FIGS. 16K and 16L illustrate and embodiment of another bandage design in accordance with the disclosure;

FIG. 16M illustrates an embodiment of another bandage design in accordance with the disclosure;

FIG. 16N illustrates an embodiment of another bandage design in accordance with the disclosure;

FIG. 16O illustrates an embodiment of another bandage design in accordance with the disclosure;

FIG. 16P illustrates an embodiment of another bandage design in accordance with the disclosure;

FIG. 16Q illustrates an embodiment of another bandage in accordance with the present application;

FIG. 16R illustrates an embodiment of another bandage in accordance with the present application;

FIG. 16S illustrates an embodiment of another bandage in accordance with the present application;

FIG. 16T illustrates an embodiment of another bandage in accordance with the present application;

FIG. 16U is a cross-sectional view of the bandage of FIG. 16T;

FIG. 16V illustrates an embodiment of another bandage in accordance with the present application;

FIG. 16W illustrates an embodiment of another bandage in accordance with the present application;

FIG. 16X is a cross-sectional view of the bandage of FIG. 16W;

FIG. 16AA is a bottom view of an embodiment of a bandage in accordance with the present disclosure, according to some embodiments;

FIG. 16BB is a bottom view of an embodiment of a bandage in accordance with the present disclosure, according to some embodiments;

FIG. 16CC is a bottom view of an embodiment of a bandage in accordance with the present disclosure, according to some embodiments;

FIG. 16DD illustrates an embodiment of another bandage design in accordance with the present disclosure, according to some embodiments;

FIG. 16EE illustrates an embodiment of another bandage design in accordance with the present disclosure, according to some embodiments;

FIG. 16FF illustrates a therapy device using a mounting system compatible with the bandage design of FIG. 16DD as disposed on a user;

FIG. 16GG illustrates the bandage design of FIG. 16DD as disposed on a user;

FIGS. 17 and 18 illustrate embodiments of support structures for use with the present application;

FIGS. 19A-19C illustrate another housing in accordance with the present disclosure, according to some embodiments, the housing configured to be received about a user's shoulders;

FIGS. 19D-19H illustrate alternative housing embodiments in accordance with the present disclosure, according to some embodiments, the housing configured to be embodied as multiple pieces;

FIGS. 20A and 20B illustrate alternative clothing mounting arrangements for the therapy device of the present disclosure, according to some embodiments;

FIG. 21A illustrates an exemplary embodiment of a computing device in accordance with the present disclosure, according to some embodiments;

FIG. 21B illustrates and exemplary screen view of a program displayed on the exemplary computing device of FIG. 21A in accordance with the present disclosure, according to some embodiments;

FIG. 22 illustrates a flow diagram for a software program in accordance with the present disclosure, according to some embodiments;

FIG. 23A illustrates a therapeutic frequency curve for the therapy provided by an exemplary embodiment of the present disclosure, according to some embodiments;

FIG. 23B illustrates a timed therapy sequence in accordance with an embodiment of the present disclosure, according to some embodiments;

FIG. 24A illustrates a patient's blood pressure reading over a twenty-four hour period, showing a hypertensive patient; and

FIG. 24B illustrates the blood pressure of the patient of FIG. 24A after receiving therapy in accordance with the device and methods of the present disclosure, according to some embodiments.

### DETAILED DESCRIPTION

The following detailed description illustrates embodiments of the disclosure by way of example and not by way of limitation. The description clearly enables one skilled in the art to make and use the disclosure, describes several embodiments, adaptations, variations, alternatives, and uses of the disclosure, including what is presently believed to be the best mode of carrying out the disclosure.

This written description uses examples to disclose the disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

In accordance with the present disclosure, according to some embodiments there is provided devices and methods for the treatment of hypertension. The device of the present disclosure, according to some embodiments is configured to be detachably attached to a user, wherein the device is aligned with a bone of the user's body. Once affixed to the patient, the device can be activated either: manually or remotely, through a wireless or wired connection, through the use of a software program running on a computing device or a software program running on a processor within the device. The activation may be timed to coincide with a patient's sleep pattern, such that therapy is provided by the device to the patient in the evening and again in the morning prior to the patient waking up. It is believed that providing therapy during a sleep cycle is beneficial to some embodiments of the present invention. Further still, the therapy providing device may “learn” the user's sleep cycle, and therefore automatically adjust the therapy start time as well as the duration of the therapy cycle or adjust/alter the pause time between the two or more therapy cycles when two or more therapy cycles are desired. Further still, the therapy providing device may be remotely activated by another person, for example a health care provider.

Additionally, the therapy providing device may be coupled to a wired or wireless remote, such as fitness tracking band (FITBIT, etc.), a watch worn by the user, a tablet such as an IPAD, or a cell phone such as an iPHONE, such that the band, watch or phone may be utilized to activate the therapy providing device. The therapy providing device may be activated remotely by the charging base 570, either through a button press on the charging base 570 or in response to a program running on a processor of the charging base 570 or in response to a remote signal received by the charging base 570.

In accordance with embodiments of the present disclosure the device is detachably attached to a patient's tissue and is intended to engage a portion of the patient's skeletal frame or thorax, particularly the clavicle area and clavicle. It shall be understood that although the present disclosure, according to some embodiments is described in reference to the collar bone or clavicle, it shall be understood that this should not be limiting in any manner. As described above, in a preferred embodiment the methods and devices of the present disclosure, according to some embodiments utilize the clavicle. However, the methods and devices of the present disclosure, according to some embodiments may be utilized with other dermal bones such as the skull, jawbone, knee cap (patella) or non-dermal bones such as the wrist bone, ribs, scapula. Methods and devices of the present disclosure, according to some embodiments can also be used on any portion of the body such as on the thorax or any other portion of the body containing somatosensory sensors such as proprioceptors, nociceptors, mechanoreceptors or thermoreceptors. Dermal bones are unique in that dermal bone does not form from cartilage first and then calcify. Dermal bone is formed within the dermis and it grows by accretion only; that is, the outer portion of the bone is deposited by osteocytes. Dermal bones have been utilized to transmit sound for other devices such as in hearing aids.

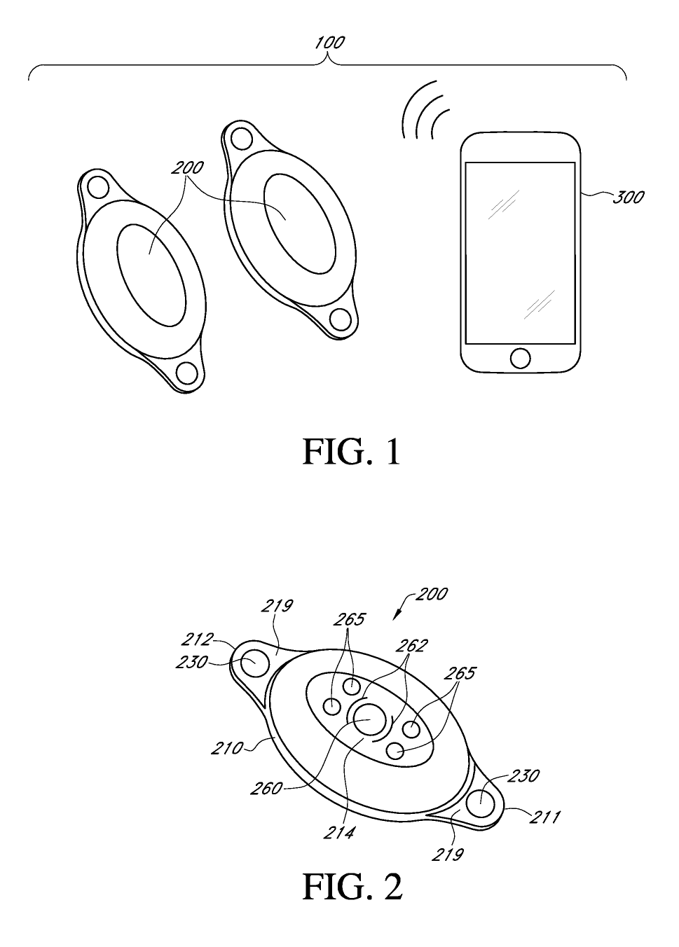

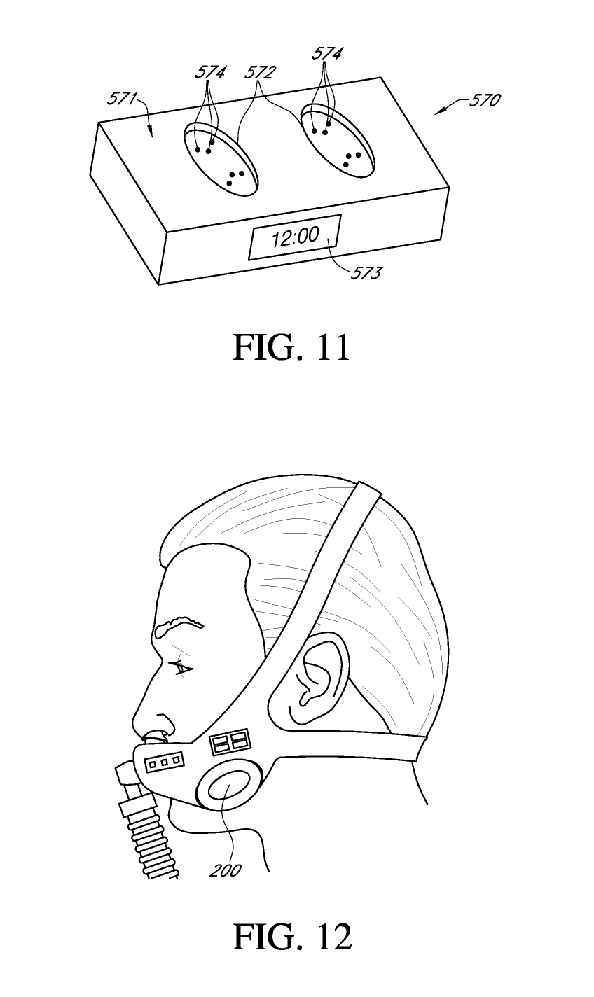

Referring now to FIG. 1, there is shown the therapy providing system 100 in accordance with the present disclosure, according to some embodiments. As shown in FIG. 1, the therapy providing system 100 in accordance with the present disclosure, according to some embodiments may include a pair of therapy providing devices 200 and optionally a computing device 300. The therapy providing system 100 may further include a charging/storage system 570 as will be described in detail below with reference to FIG. 11. Additionally, the therapy providing device 200 may further include an integrated or separate attachment system to detachably attach the system 100 to a user's skin as will be described in greater detail below.

As shown in FIG. 1, the computing device 300 in one aspect is configured to communicate with the therapy providing device 200 through a wireless communication protocol such as through the use of WIFI, BLUETOOTH, ZIGBEE, RFID, NFC, ANT+, cellular, infrared or other known wireless communication protocols. Alternatively, the computing device 300 and the therapy providing devices 200 may be communicatively coupled together using a physical connection such as an electrical wire, a plurality of electrical wires, electrical cable, fiber optic or using other known physical connections capable of transmitting signals, including data and/or power, between the therapy providing devices 200. As shown in FIG. 1, it is contemplated that the methods of use in accordance with the present disclosure, according to some embodiments would utilize two therapy providing devices 200 as shown. If two therapy providing devices 200 are utilized, they are intended to be disposed on a user about or adjacent to the user's left and right clavicle. The therapy providing devices may be disposed along the length of the user's clavicle, i.e. horizontal, or may be disposed generally perpendicular to the user's clavicles. In accordance with the present disclosure, according to some embodiments a single therapy providing device 200 may be utilized for treatment according to the present disclosure, according to some embodiments, or multiple therapy providing devices 200 may be utilized for therapy. When two or more therapy providing devices 200 are utilized, the devices 200 can be communicatively coupled together utilizing a physical connection or utilize a wireless connection such as those described above.

Referring now to FIG. 2, there is shown an isometric view of a therapy providing device 200. As shown in FIG. 2, the therapy providing device 200 includes a housing 210. The housing 210 defined by a proximal end 212 and a distal end 211, and first surface 214 and a second surface 215 (not shown), the proximal end, distal end and first and second surfaces and defining a volume therebetween, wherein the volume includes additional structures and components as will be described below. As shown in FIG. 2, the first surface 214 may include a power button 260, at least one LED indicator light 262, and at least one pair of charging pins 265. The multiple charging pins 265 may be included on the therapy providing device 200, whereas the multiple pins 265 are disposed symmetrically about an axis (not shown) passing through the power button 260. Placement of the charging pins 265 about an axis extending through the power button 260 allows for the therapy providing device 200 to be placed within a charger without care as to orientation as each side of the therapy providing device 200 includes charging pins 265, such that the therapy providing device 200 will engage the changing pins in the charging station in either orientation. Additional details with regard to a charging station/base will be described in greater detail below with reference to FIG. 11. Additionally, the housing 210 may further include magnets 230 or a metallic material disposed within recesses formed in the first and second ends 211,212 of the housing 210. Alternatively, the magnets 230 may be integrally formed with the housing 210 during a manufacturing process such as injection molding, machining or 3D printing or other additive manufacturing process. Further still, the first and second ends 211,212 of the housing may be embodied as separate flexible arm components which are configured to be associated with the housing, wherein the flexible arms 219 are configured to include magnets or metallic members. Such an embodiment will be described in further detail below.

The housing 210 may be formed of multiple pieces which may then be assembled using known assembly methods such as glue, ultrasonic welding, heat welding, rotational welding, snap-fit construction, use of fasteners such as screws or pins, or the like. In accordance with the disclosure, the housing 210 may be formed of two pieces or multiple pieces, wherein one section of the housing 210 includes all sides except the second surface 215, thereby forming a shell into which the components can be disposed, then the second surface 215 could be attached to the other portion of the housing 210 to form the therapy providing device 200. The housing 210 may be constructed of biocompatible materials such as polymers, plastics, fabrics, ceramics, or metals. The housing 210 may be formed using manufacturing processes such as machining, injection molding, rotary molding, 3-d printing, vacuum forming, deep drawing or the like. In accordance with the disclosure, the materials utilized in construction of the housing 210 of the therapy providing device 200 shall be chosen such that the materials have good biocompatibility as it is intended that the therapy providing device 200 may be placed in skin contact during use, where in certain usages the skin contact may be for a prolonged time.

Further still, it is contemplated that the therapy providing device 200 may be wrapped or encased with a biocompatible membrane. An example of a suitable membrane is available from 3M and sold under the tradename of TEGADERM.

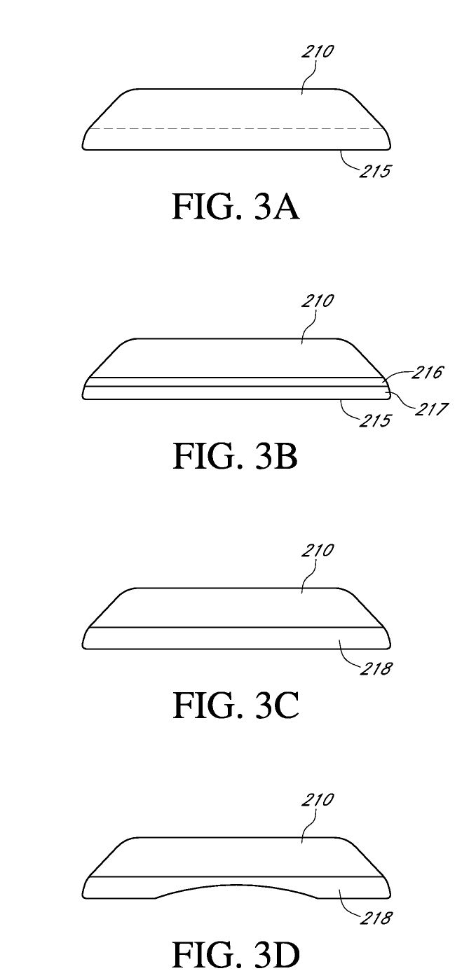

Referring now to FIGS. 3A-3D there is shown an exemplary embodiment of the second surface 215 in accordance with the present disclosure, according to some embodiments. As shown in FIG. 3A, the second surface 215 may be formed as a planar surface. Referring now to FIG. 3B, in this figure, the second surface 215 is formed of multiple pieces, wherein one component 216 is configured to be received by the other portion of the housing and the second component 217 is configured to be received by a user's tissue, this portion 217 may be formed of a more pliable or conformable material than the first component 216, wherein the more pliable material 217 may conform or shape to the user's anatomy more readily. In accordance with the disclosure, the second component 217 may be formed of a compliant material such as and open or closed cell foam material, such that when the therapy providing device 200 is disposed upon a user for therapy, the compliant foam surface conforms to the user's anatomy. Additionally, the materials selected may be chosen such that they are anti-microbial and/or anti-bacterial.

Referring now to FIGS. 3C and 3D there is shown another exemplary embodiment wherein the second surface 218 is shown having a first thickness, wherein the material of which the first surface 218 is formed is selected such that the material may be shaped or contoured to be received by a patient's skin, particularly in an area adjacent the patient's clavicle. The shaped surface may be in the form of a concave shape. Further still, the material 218 of FIGS. 3C and 3D may be selected such that the material defines a deformable structure, such that when the housing is placed over the patient's clavicle the housing conforms to the patient's anatomy as shown in FIG. 3D. In yet another embodiment, a portion of the housing may be custom formed to each individual user through the application of heat, whereby the housing or a portion of the housing is heated and then pressed onto the patient, the heated portion of the housing conforming to the patient's anatomy, or heated and molded by through an application of force. Further still, the housing may be manufactured in response to the patient's anatomy, in this embodiment, the patient's body is scanned with a 3D scanner or multiple 2D pictures are taken and processed to create a 3D model of the patient's anatomy. The portion of the housing of the therapy providing device 200 may then be custom machined, printed or fabricated utilizing this data.

In another aspect, the second component 217 may be embodied in the form of a flexible membrane in which an expandable foam material or other types of expandable materials may be injected into. In use, the therapy providing device would be placed on the user in a chosen location, the expandable material could then be injected into the flexible membrane while the therapy providing device is held against the user. As the foam expands and cures, the second component 217 would take the shape of the user's anatomy, thereby providing a customized fit. The foam in a cured state may be flexible or rigid. Alternatively, the second component, being fabricated as a flexible membrane may be filed with a biocompatible or non-biocompatible material, examples of such materials may be saline or silicone so that the second component 217 of the housing 210 readily conforms to the user's anatomy.

Lastly, it is further contemplated that the housing includes an enlarged or thickened surface that can be ground or machined away to conform to the patient's anatomy. Further still, a mold may be taken of the patient's anatomy, whereby a housing can then be manufactured from the mold taken from the user's anatomy, thereby customizing the fit of the therapy providing device to each user.

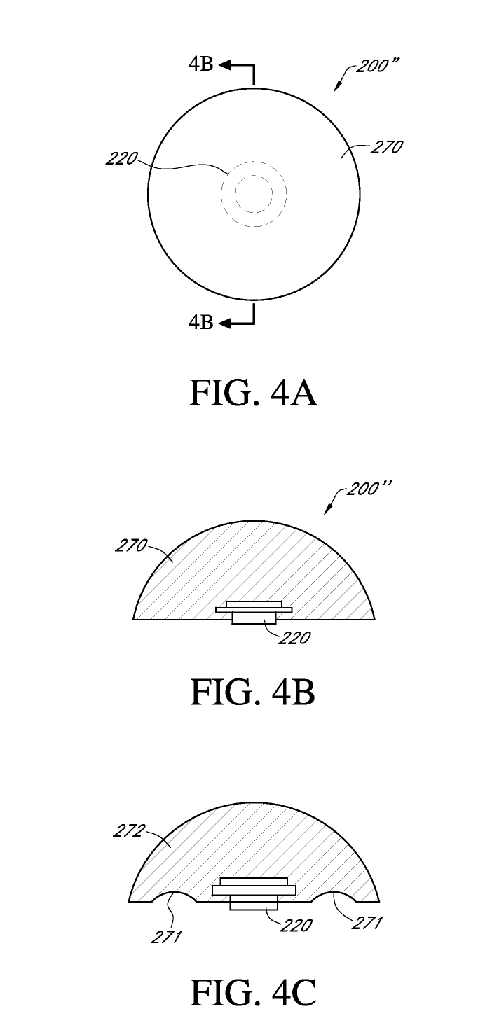

Referring now to FIGS. 4A-C there are shown additional housing designs in accordance with the present disclosure, according to some embodiments. As shown in FIG. 4A the alternative housing is formed in a generally circular fashion, wherein the housing contains additional components as will be described in greater detail below. Also as shown in FIG. 4A, the housing 270 may further include a wire or cable connection extending from the housing 270 as described above. Referring now to FIG. 4B, there is shown a cross-sectional view of the housing 270 of the therapy providing device 200″ of FIG. 4A taken about line B-B of FIG. 4A. As shown in the cross-sectional view, the alternative housing 270 is formed having a generally convex shape. Referring now to FIG. 4C there is shown a cross-sectional view of yet another alternative embodiment of a housing 272, in this embodiment the housing 272 has a generally convex shape as previously described, however, in this embodiment the housing 272 includes concave portions 271. In use, the housing 272 is placed on a user, adjacent to the user's clavicle, wherein a force can be applied to the housing 272 adjacent to each concave portion 271 forcing air out of the concave portions 271, thereby causing a vacuum to be formed thereby suctioning the housing 272 to the user's tissue. It is contemplated that the housings 270 and 272 shown in FIGS. 4A-4C may be constructed of a biocompatible flexible materials, such that the housing conforms to the user's anatomy when placed thereupon. Examples of suitable materials of which the housings 270, 272 may be formed from are: silicone, urethanes, rubber, latex, stainless steel, glass filled nylon, plastic, ceramics, TPU, TPE, gels, viscoelastic material, epoxies and the like.

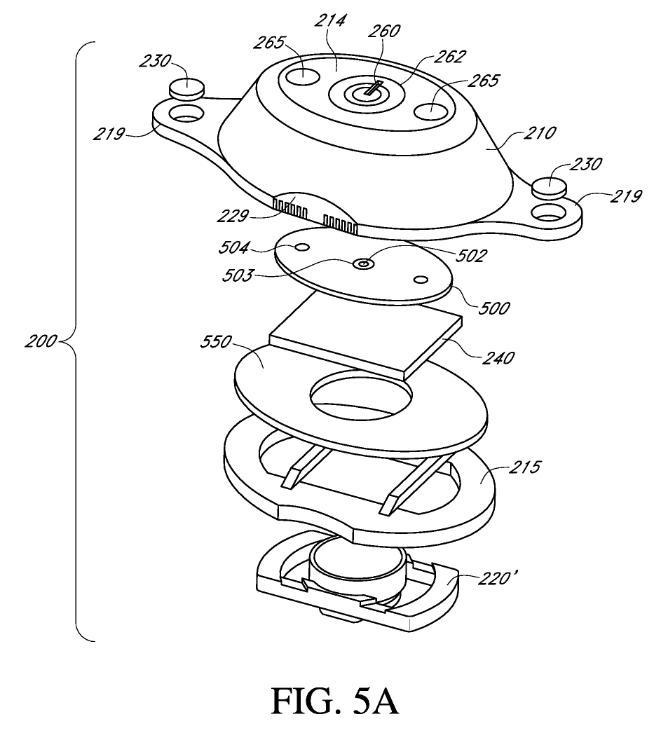

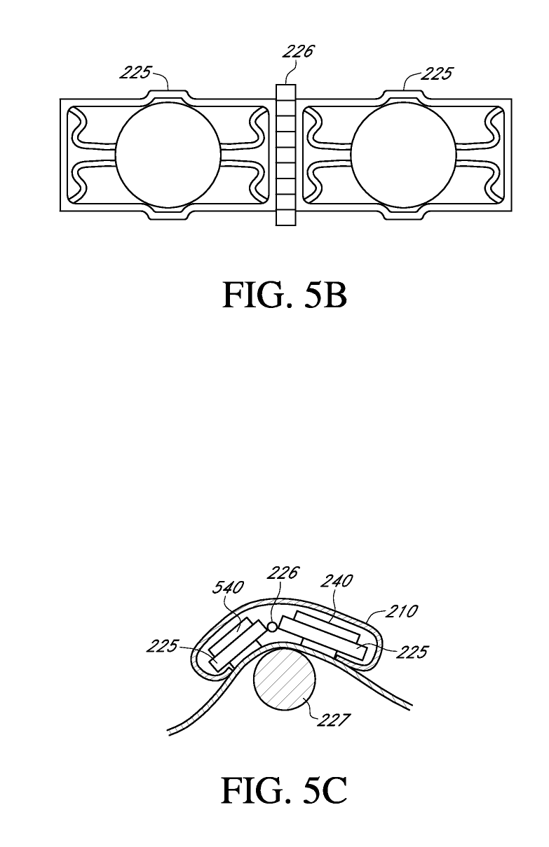

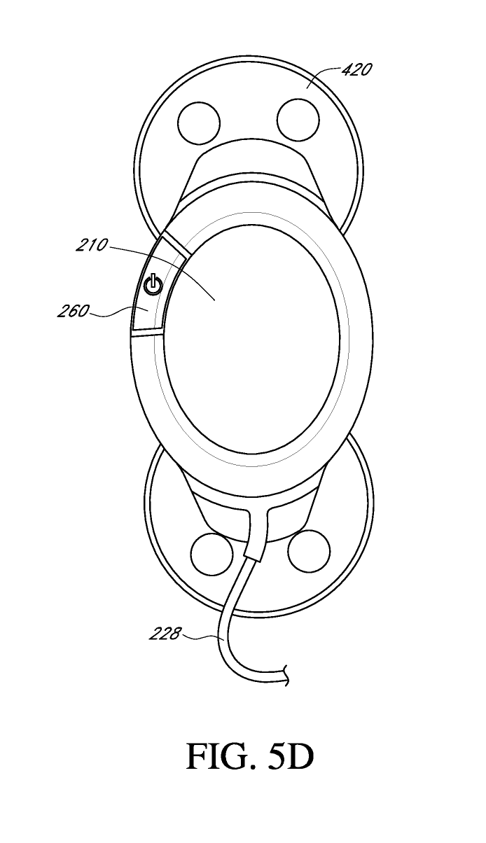

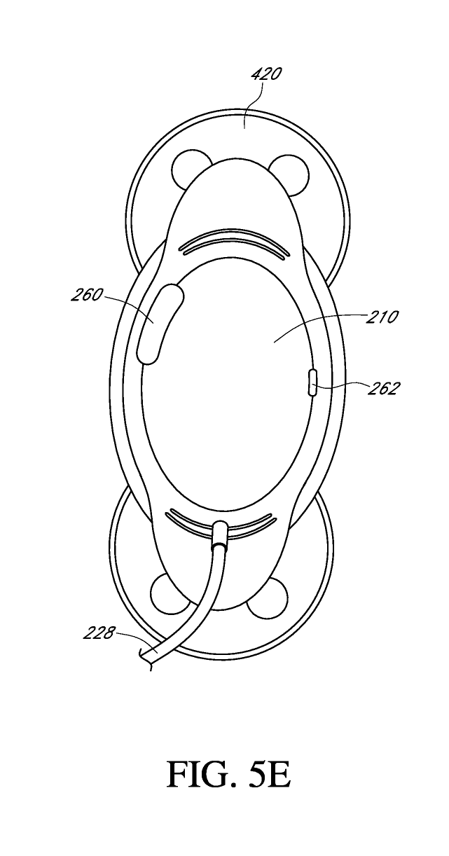

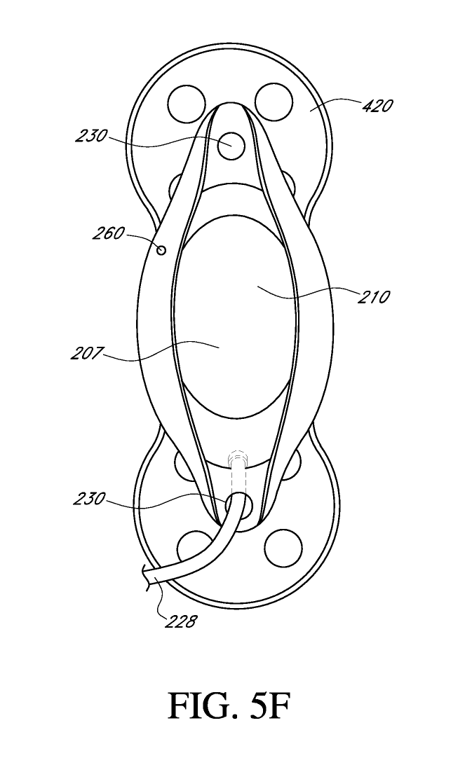

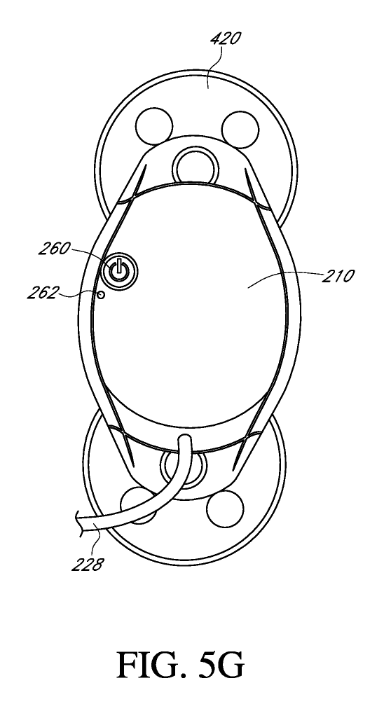

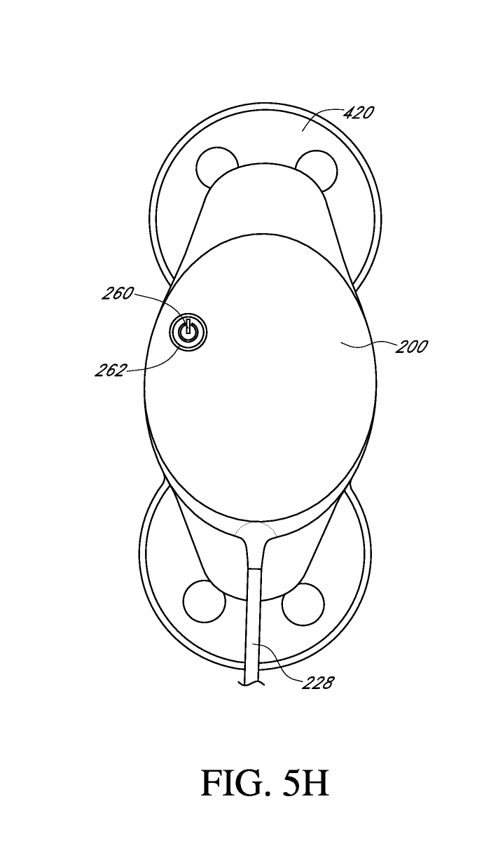

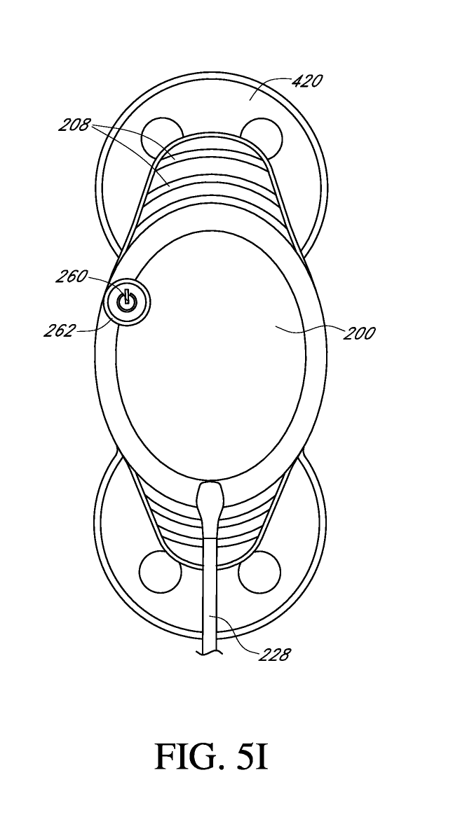

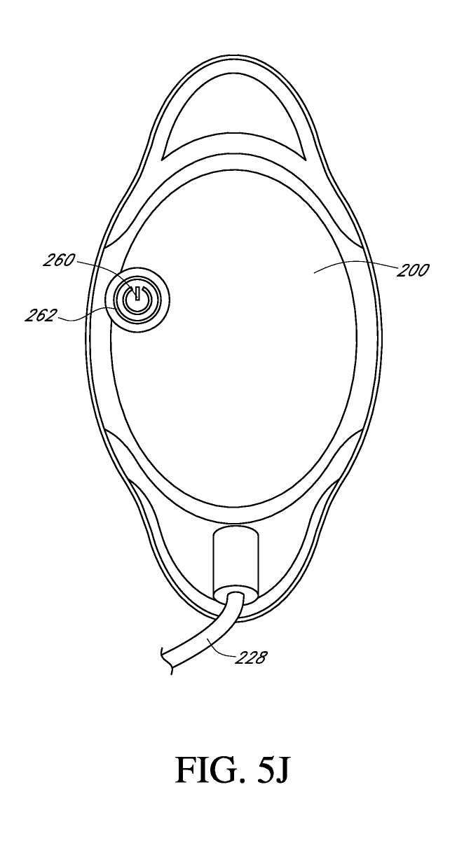

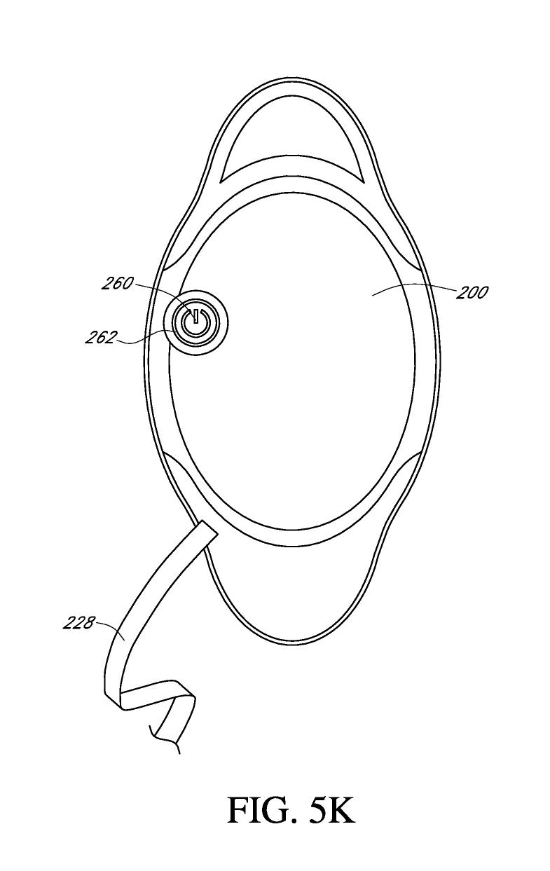

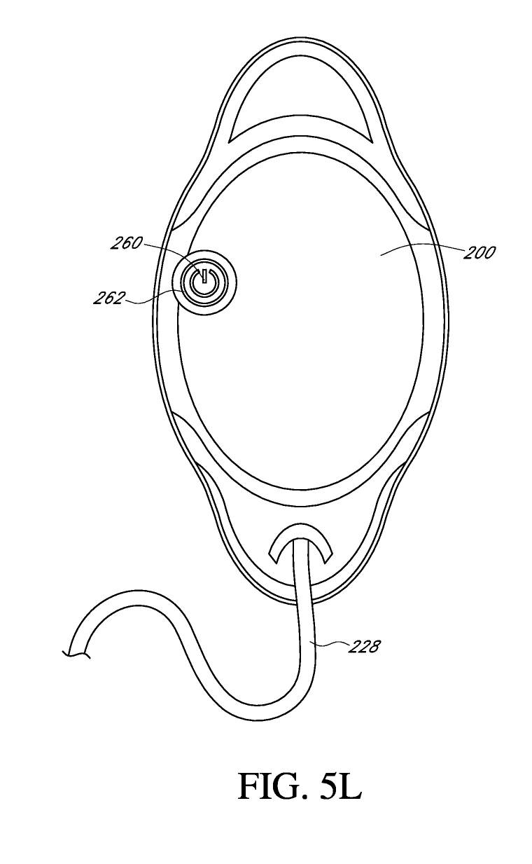

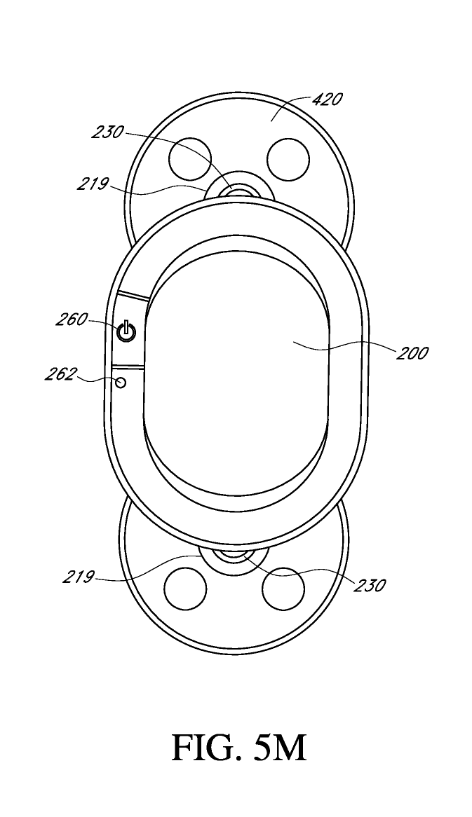

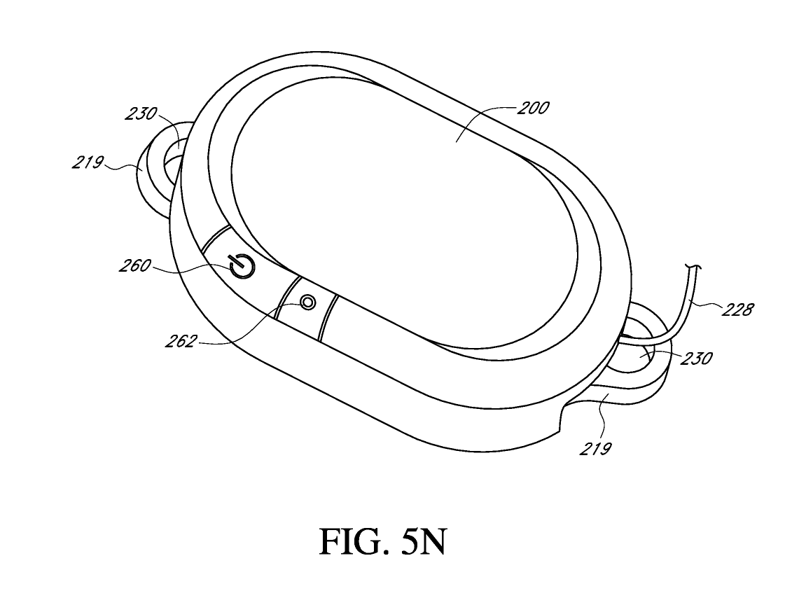

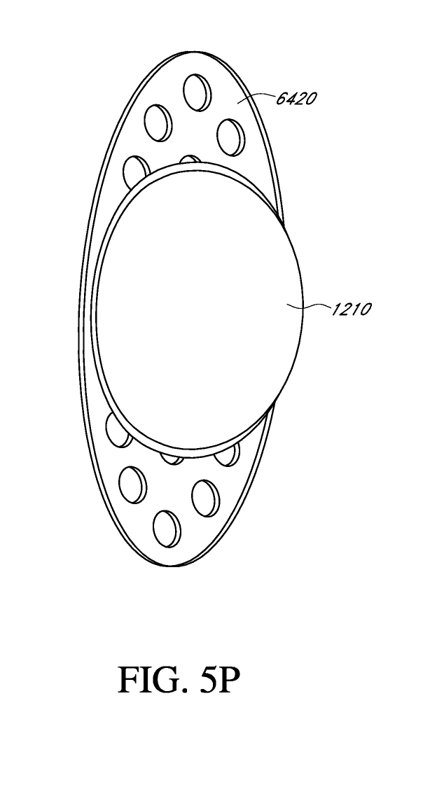

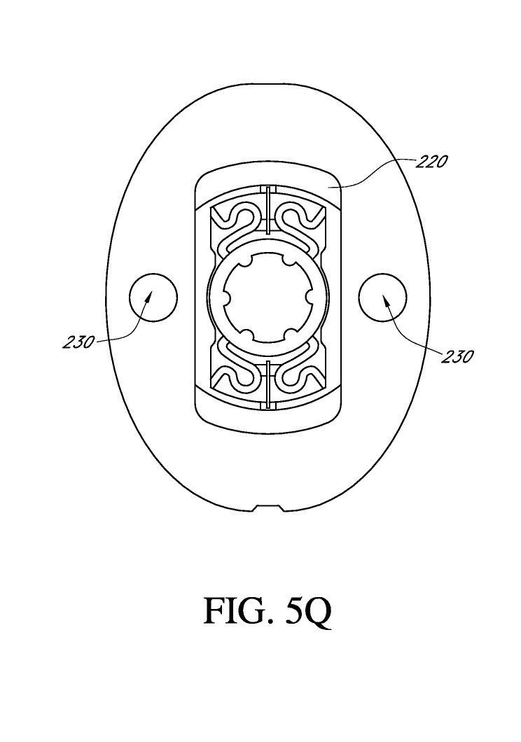

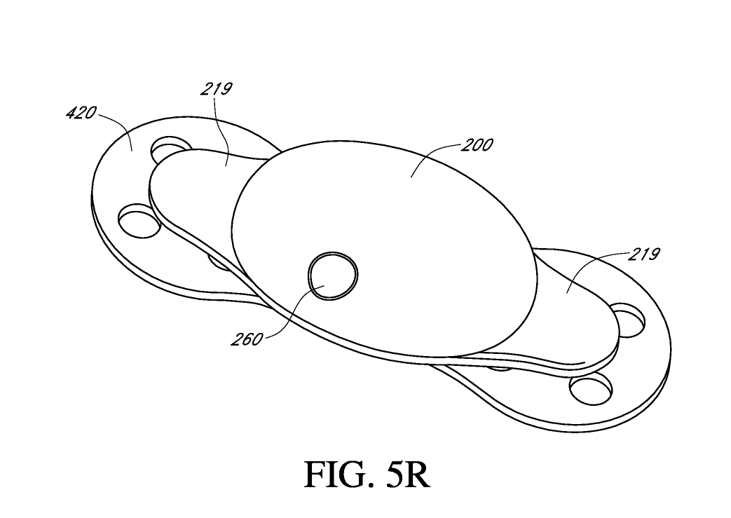

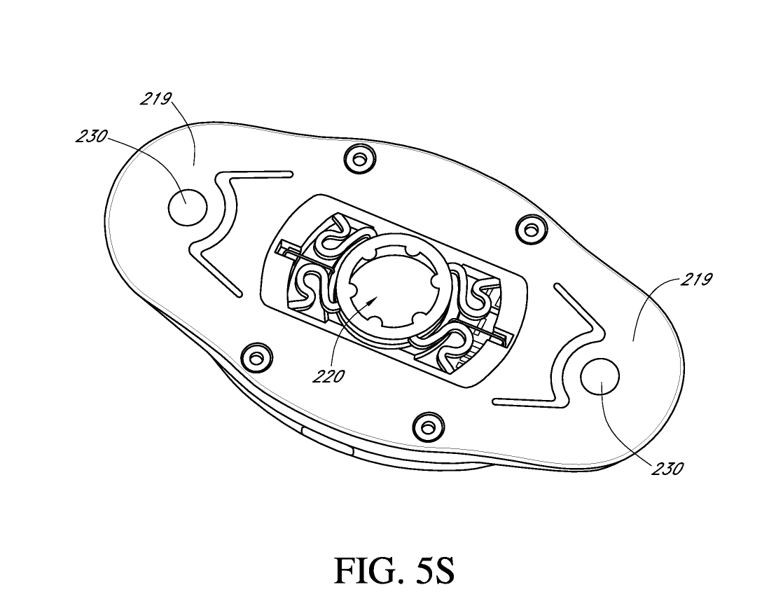

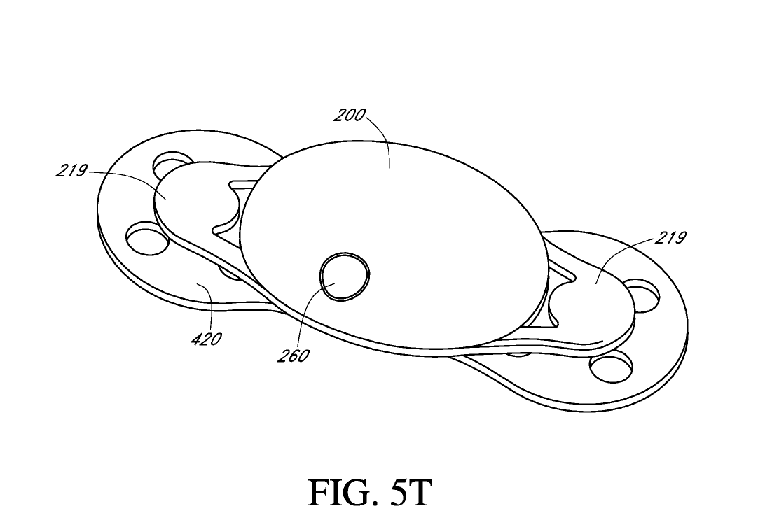

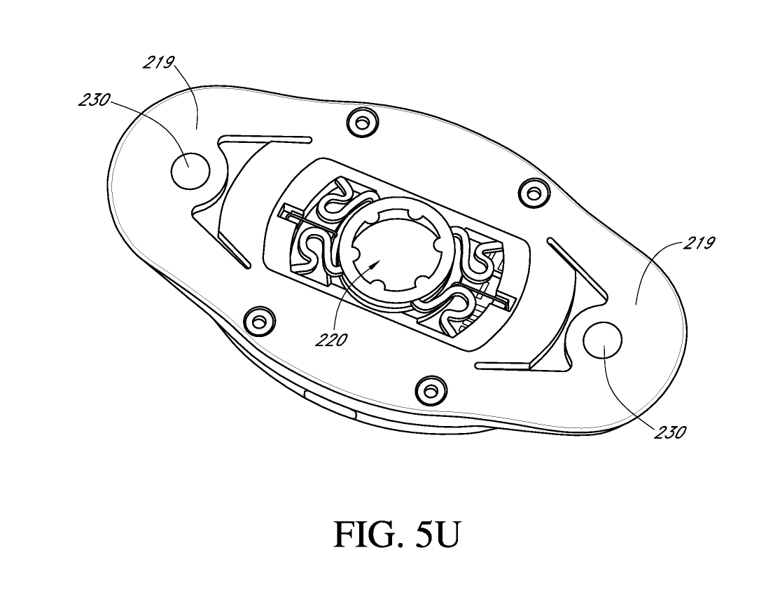

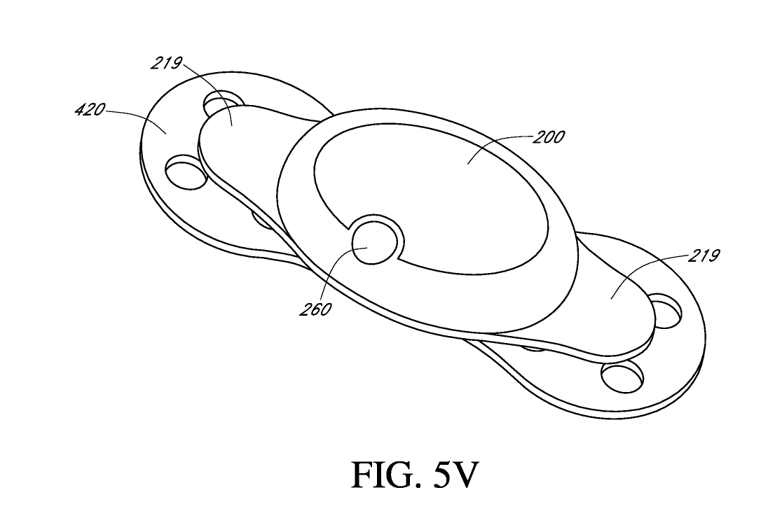

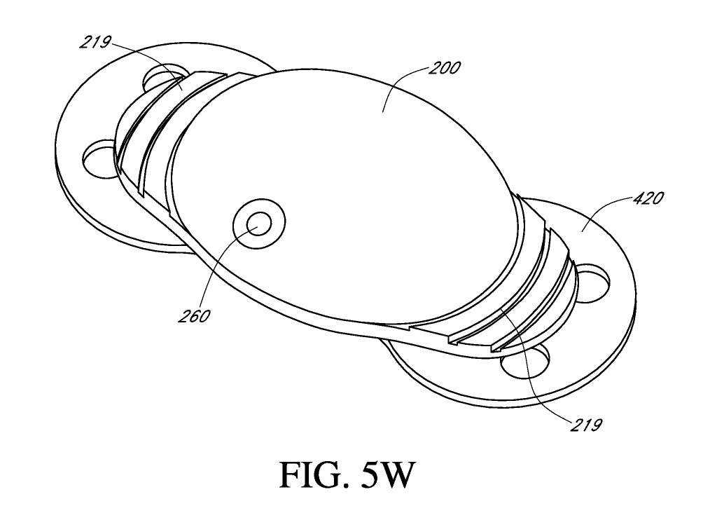

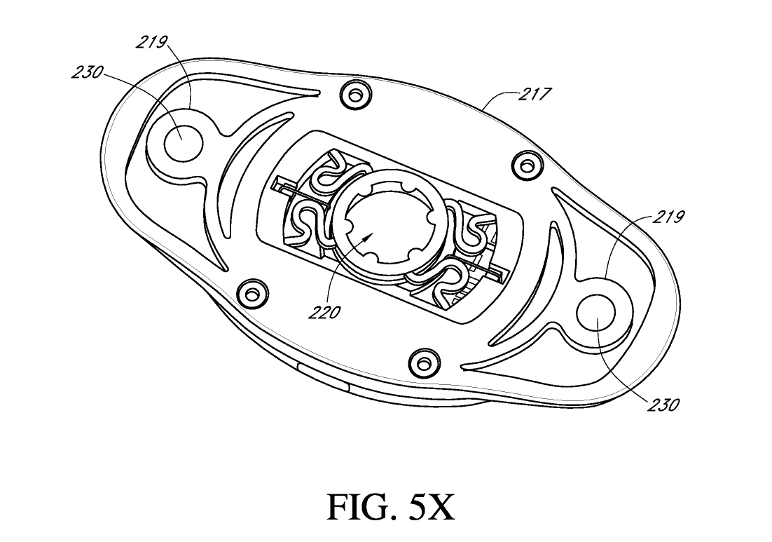

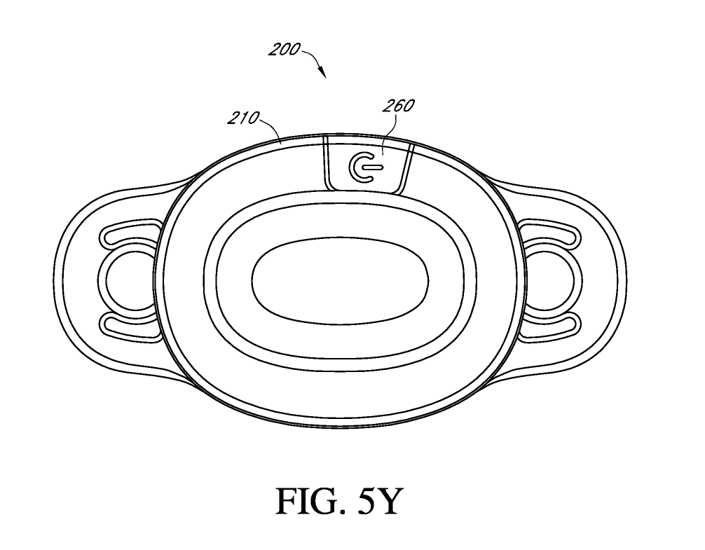

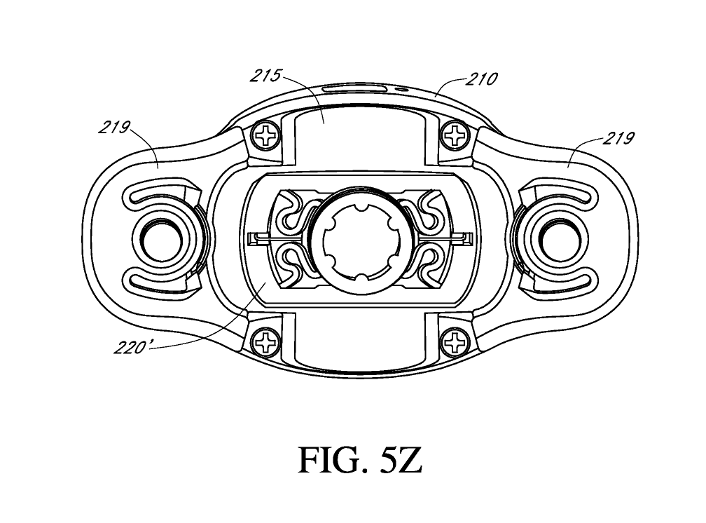

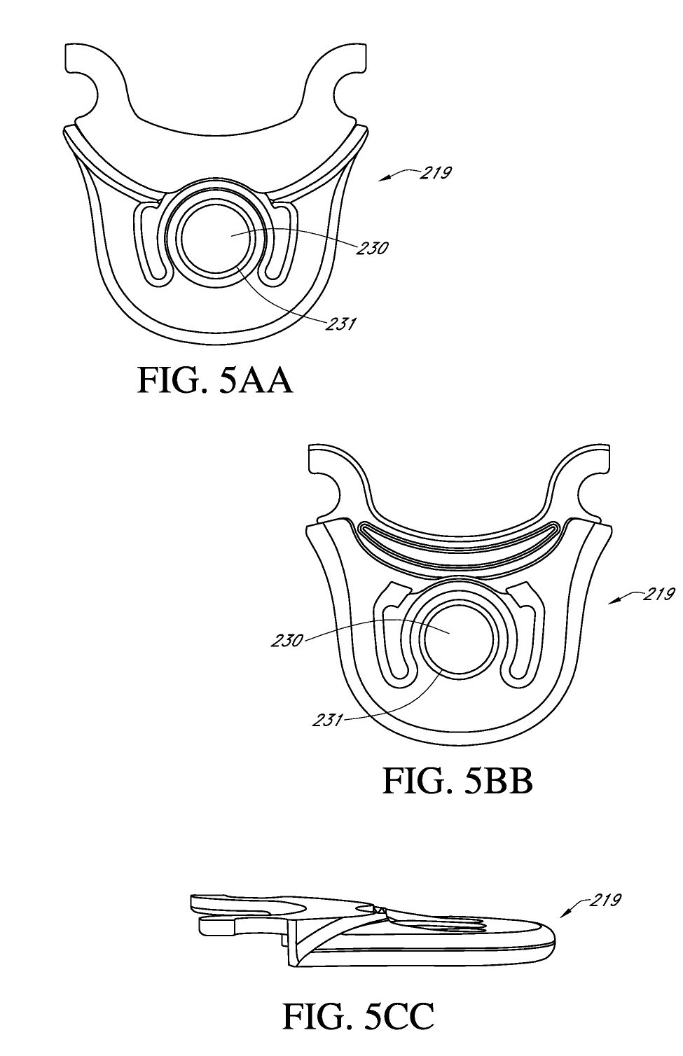

Referring now to FIG. 5A, there is shown an exploded view of a therapy providing device 200 in accordance with the present disclosure, according to some embodiments. As described above and shown in FIGS. 2-4, the therapy providing device includes a housing 210, wherein the housing includes provisions for a power switch 260 as well as provisions for LED indicators 262 and charging pins 265 as described above. The power switch 260 maybe a separate component disposed within an opening of the housing 210 or it may be embodied as a reduced thickness portion (not shown) of the housing 210 which can be formed to project slightly above the first surface 214 of the housing 210, whereby in use, a user can apply a light force to the raised portion to active a switch disposed beneath the raised portion. Forming a raised portion integral to the first surface of the housing 210 to be utilized as a switch simplifies construction, eliminates additional components, this construction also eliminates the need to form a hole within the first surface of the housing which may require sealing against liquids or particle intrusion. Further, the provisions for the light emitting diode (LED) 262 and the charging pins 265 may be in the form of openings formed within the housing to receive such items. Alternatively, the LED 262 provision may be embodied in the form of an opaque or clear section within the housing 210 during manufacture to allow light to project therethrough from a LED 503 mounted on a circuit board 500 disposed below the housing 210. Additionally, it is contemplated that the charging pins 265 may also be integrally formed during the manufacture of the housing 210. For example, if the housing 210 is manufactured using an injection molding process, the charging pins 265 could be disposed within the injection mold as an insert, whereby the charging pins 265 would be captured in the housing 210 during the molding process. Alternatively, the housing 210 can include openings for charging pins 265 to project through. Further still, the housing 210 may include openings having tapered wall portions, forming pockets within the first surface 214, thereby providing access to charging pads/pins 504 disposed on a circuit board 500 disposed below the first surface 214 of the housing 210. The housing 210 may further include an indentation 229 formed therein or a plurality of indentations 229, allowing a user to grasp the housing 210.

It is further contemplated that the power switch 260 may be disposed adjacent to the second surface of the housing 215, wherein the second surface 215 may include a reduced thickness portion adjacent the power switch 260 as described above. It is additionally contemplated that the LED(s) 262 may also be disposed adjacent to the second surface of the housing, such that they can be viewed through the second surface of the housing. The viewing angle/ability of the LED(s) may be improved through the use of a light pipe or other light transmitting or carrying technologies. Additionally, it is contemplated that the LED(s) maybe disposed close to the exterior surface of the second surface of the housing such that the portion of the second surface of the housing adjacent to the LED(s) may be thinner. The power switch and LED(s) may be mounted on a separate circuit board disposed adjacent to the second surface of the housing, this separate circuit board would be in electrical communication with the circuit board(s) as described herein. A potential benefit of placing the power switch 260 adjacent to the second surface 215 is that the user can power the therapy providing device on, then place the therapy providing device onto a bandage disposed on their body. In this position, the power switch 260 being on the bottom of the device prevents the therapy providing device from being accidentally powered off due to inadvertent presses of the power switch.

It is further contemplated that the power switch may be controlled through a program either residing within memory of the therapy providing device 200 or the computing device 300 such that after the power switch has been pressed, further presses of the power switch will not alter the state of the program. For example a single press of the power switch will cause the therapy providing device to be powered on, such that thereafter the therapy providing device will run through a therapy cycle before automatically powering off. Further still, after powering the therapy providing device on, the therapy providing device may be powered off and/or reset by connecting the therapy providing device to the charging base or a recharging wire or pressing the power switch again or holding down the power switch for a determined amount of time.

As shown in FIG. 5A, there is an exemplary embodiment the therapy device 200 further includes a first circuit board 500 and a second circuit board 550. The first circuit board 500 is disposed adjacent to the first surface 214 of the housing 210, wherein the first circuit board 500 includes a power switch component 502, at least one indicator LED 503 configured to indicate the power status of the therapy providing device 200. The first circuit board 500 further includes charging pins/pads 504, wherein the charging pins/pads may be configured to project through the housing 210 as described above, or alternatively, the housing 210 may include openings formed therein to access the charging pins/pads 504. The power switch 502 may be embodied as a physical switch, such as a slide switch, or may be embodied as a touch sensitive or capacitive sensitive switch, or may be embodied as a pressure sensitive switch. The first circuit board 500 further includes a connector (not shown) which is configured to electrically connect the first and second circuit boards. The connector may be embodied as solder holes in which wires can be disposed into or may be embodied in the form of a plug or header assembly, wherein the plug/header are configured to accept a cable, wire, ribbon cable or a flexible PCB to facilitate electrical communication between the boards. The first and second circuit boards may be constructed of known materials and methods, whereby the boards may be hard rigid board assemblies or may be constructed using flexible board manufacturing technologies.

Although, it is described above that the present disclosure, according to some embodiments utilizes two circuit boards, this should not be considered limiting in any manner, it is contemplated that the electronic components of the present disclosure, according to some embodiments may be embodied on a single circuit board or on multiple circuit boards. As described herein, the circuit board or boards including any components connected thereto may be referred to as an electronics module 540. Further still, when embodied as a pair of therapy providing devices, the electronic components maybe fully contained within one of therapy providing devices or the electronic components can be disposed between the two devices. It is further contemplated that the device may include a charging port in place of the charging pins described herein. The charging port can be configured as a custom design or utilize off the shelf components such as USB, mini-USB, micro-USB designs. Additionally, the charging pins may be replaced by a non-contact charging system, such as an inductive charging system.

FIGS. 5A-5CC illustrate additional housing embodiments for the therapy providing device 200 in accordance with some embodiments of the present invention wherein reference numbers are utilized to denote common structures between the various designs.

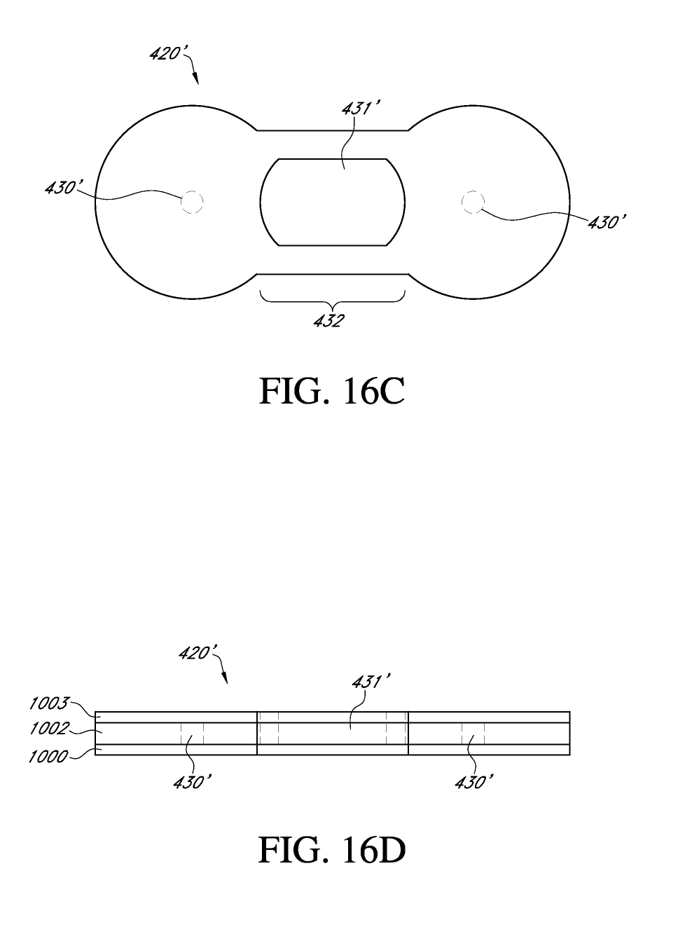

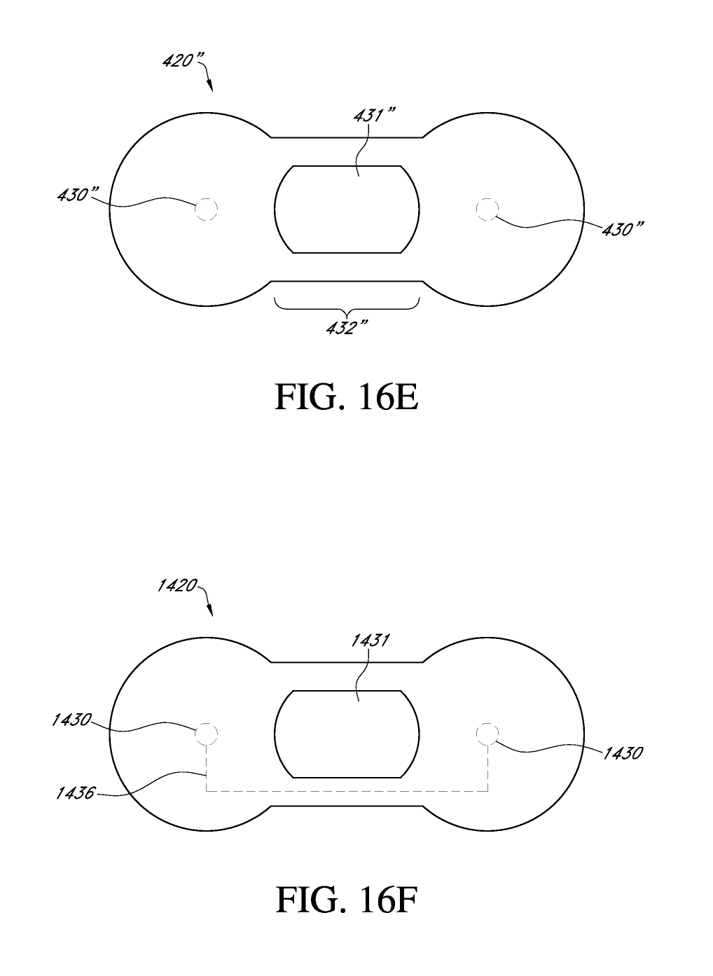

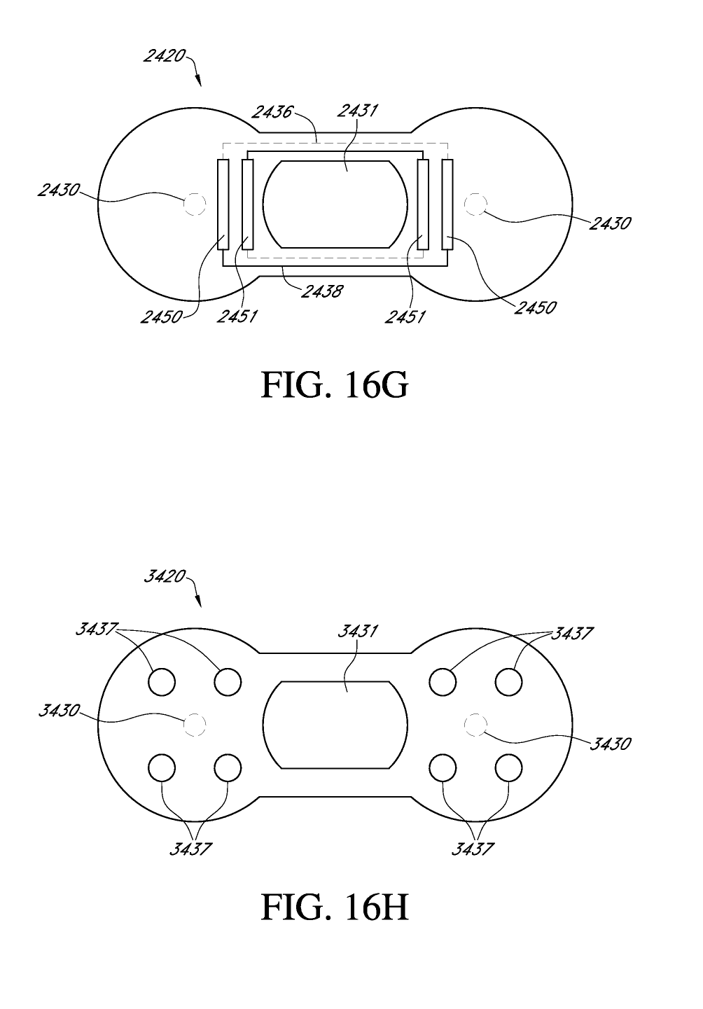

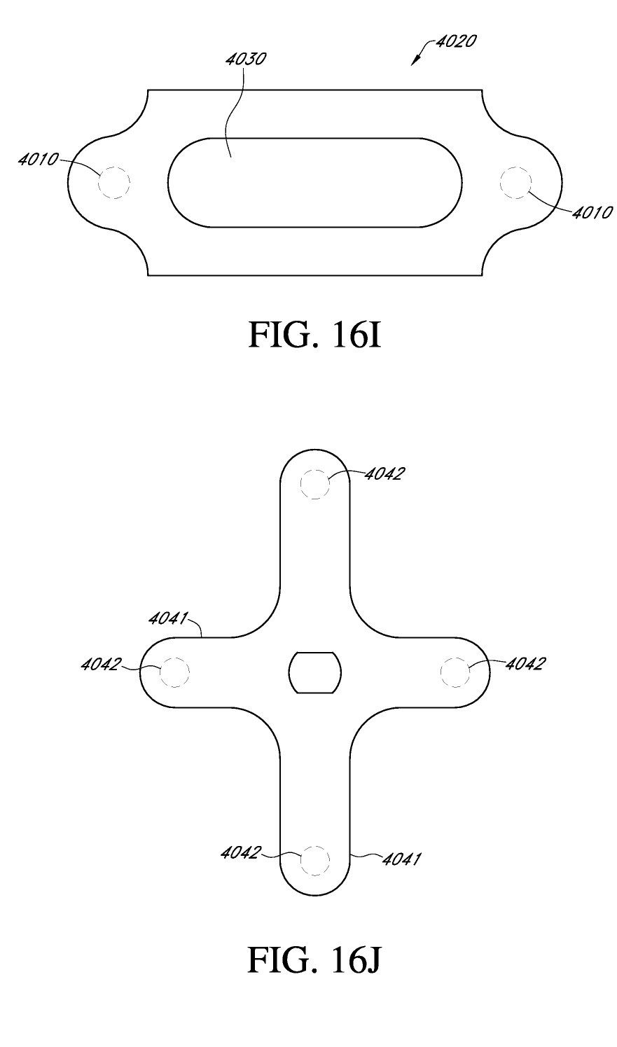

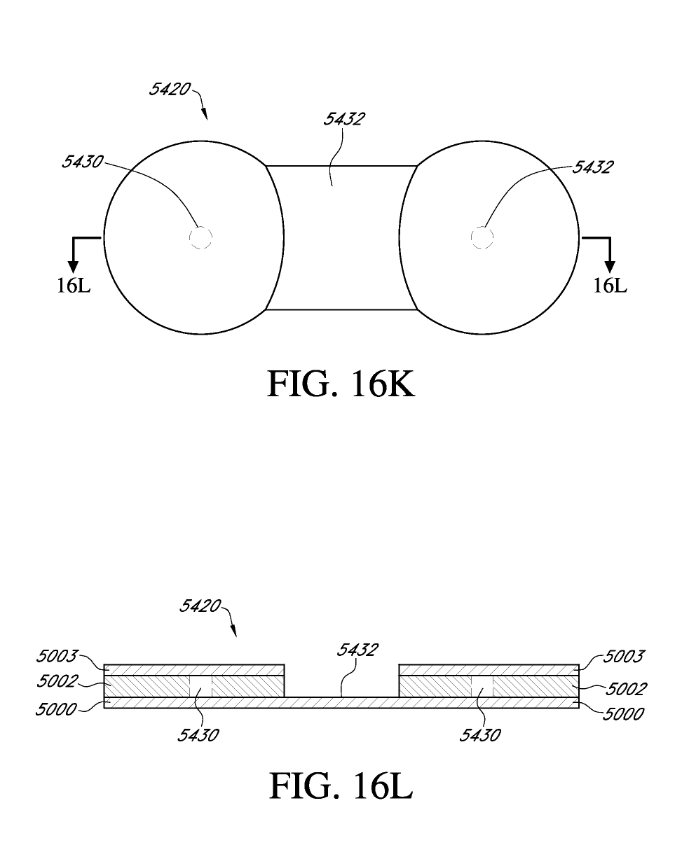

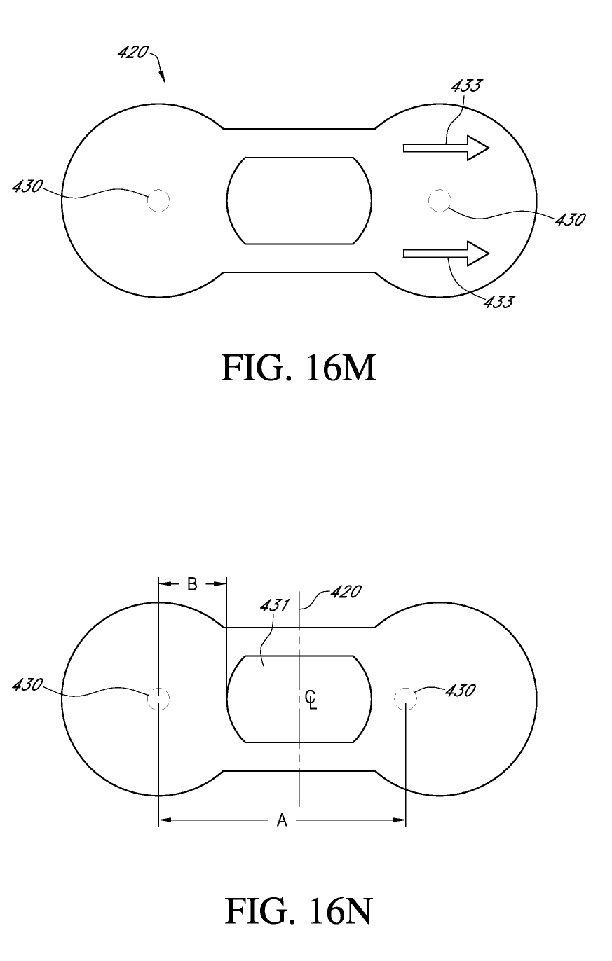

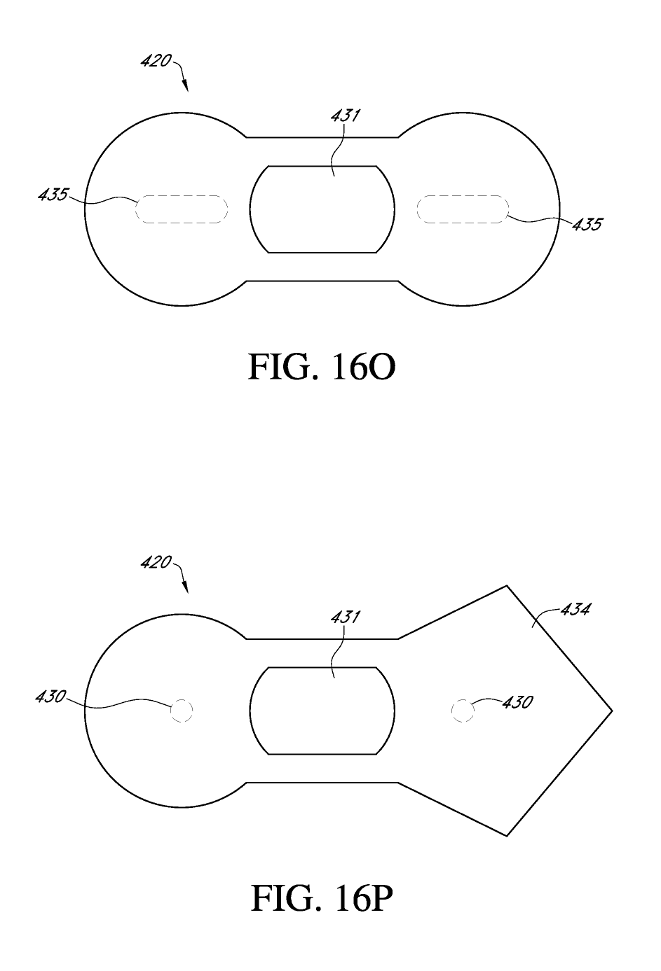

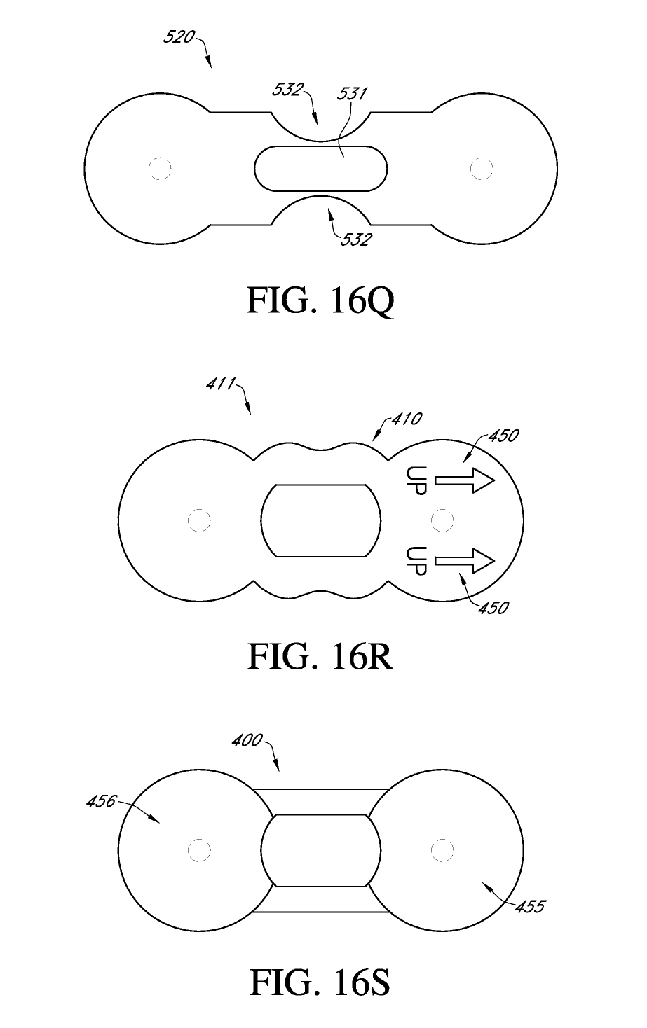

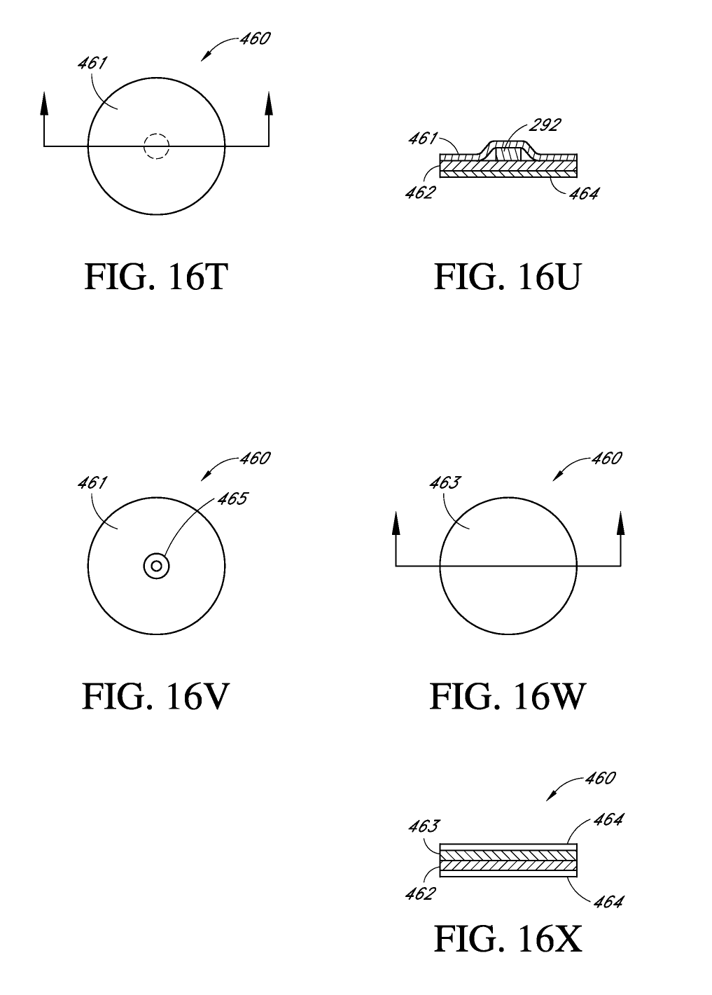

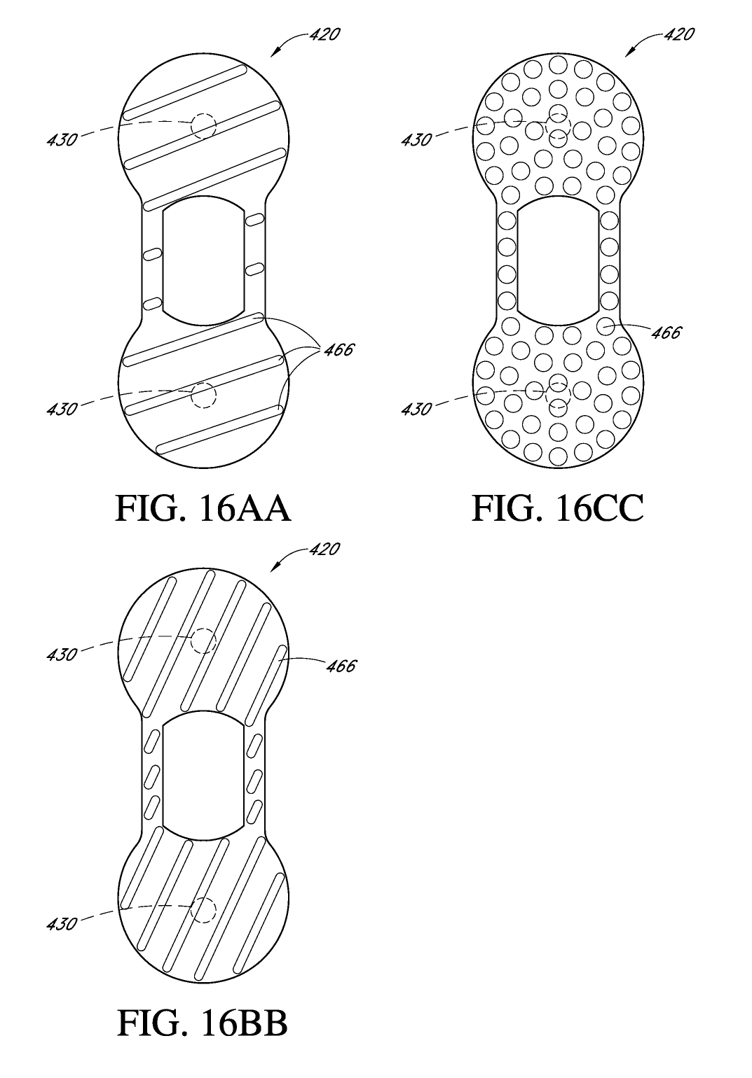

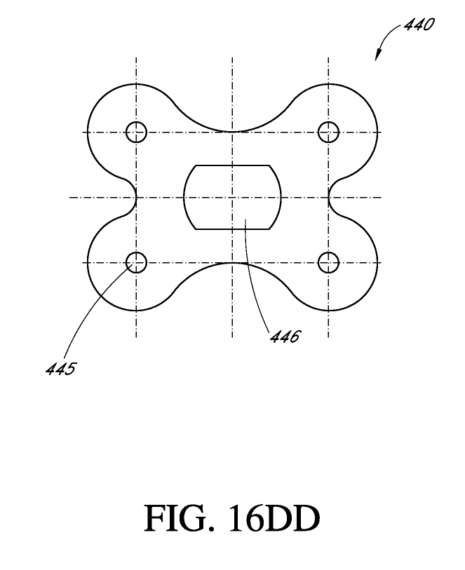

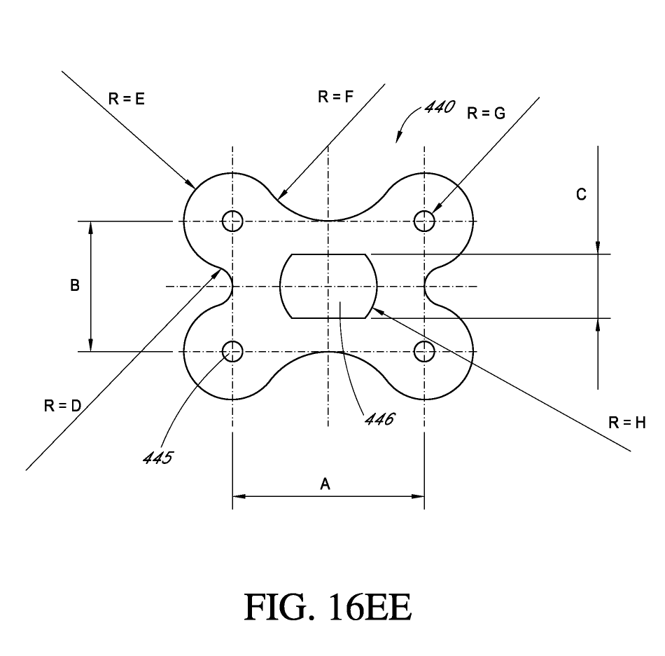

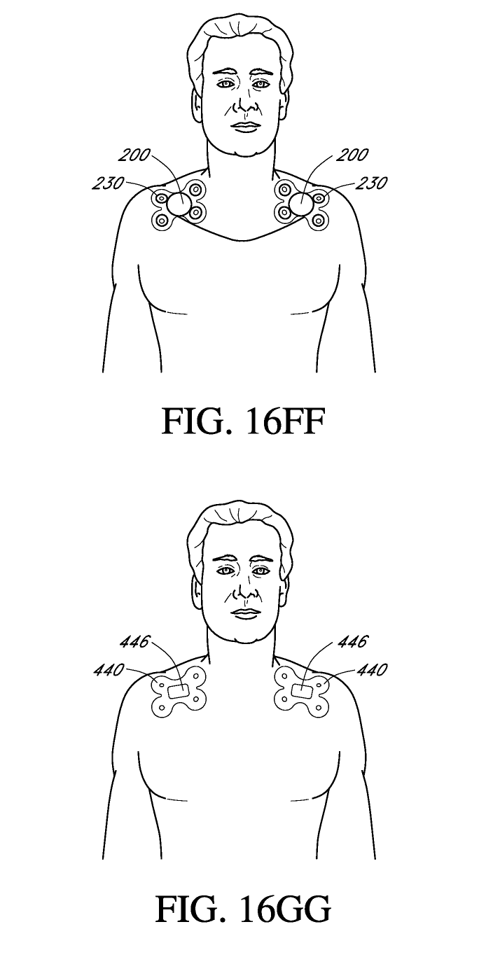

Referring now to FIGS. 5B and 5C there is shown another embodiment of a therapy providing device 205 in accordance with the present disclosure, according to some embodiments. As shown in FIG. 5B, the therapy providing device 205 utilizes a pair of driver assemblies 225, wherein as shown in FIG. 5B, the driver assemblies are haptic speakers. The driver assemblies 225 are flexibly coupled together through a hinge or flex member 226. The hinge or flex member maybe embodied as a multi-piece hinge member or as a unitary member which is configured to flex, such as a living hinge member as known to one skilled in the art. Referring now to FIG. 5C, there is shown a partial cross-sectional view of the therapy providing device 205 in accordance with the disclosure as disposed about a user's clavicle 227 in accordance with the disclosure. The therapy providing device 205 may be disposed onto the user's tissue as described herein through the use of a bandage assembly such as that shown and described in FIGS. 16K and 16L. As shown in FIG. 5C, the therapy providing device 205 includes the driver assembly 225 as shown in FIG. 5B, an energy source 240 as described herein and at least one electronics module 540 as described herein.

Referring now to FIG. 5D there is shown an additional embodiment of an alternative housing 210 in accordance with the present disclosure, according to some embodiments. As shown in FIG. 5D the alternative housing design includes a power switch 260 and a cable 228 extending from one end of the housing. The power switch 260 may further include at least one light pipe or reduced thickness portion to allow for the transmission of light from a LED. The cable 228 may be utilized to couple the therapy providing device to another therapy providing device or to a computing device 300 as described herein. It is contemplated that the housing shown in FIG. 5D may not include the cable 228 or the provision for the cable 228, wherein the therapy providing device would utilize wireless communication as described herein. As shown in FIG. 5D, the therapy providing device 210 is configured to be detachably attached to a bandage 420 as described in accordance with the present disclosure, according to some embodiments.

Referring now to FIG. 5E there is shown an additional embodiment of an alternative housing 210 in accordance with the present disclosure, according to some embodiments. As shown in FIG. 5E the alternative housing design includes a power switch 260, LED 262 and a cable 228 extending from one end of the housing. The cable 228 may be utilized to couple the therapy providing device to another therapy providing device or to a computing device 300 as described herein. It is contemplated that the housing shown in FIG. 5E may not include the cable 228 or the provision for the cable 228, wherein the therapy providing device would utilize wireless communication as described herein.

Referring now to FIG. 5F there is shown an additional embodiment of an alternative housing 210 in accordance with the present disclosure, according to some embodiments. As shown in FIG. 5F the alternative housing is comprised of multiple pieces 206, 207 and 215 (not shown). In this embodiment, it is contemplated that one portion 206 of the housing may be constructed of a pliable material, wherein the second portion 207 of the housing is then adapted to be received and retained by the pliable portion 206. As shown in FIG. 5F, the portion 206 includes housing extensions or arms 219 including magnets 230 as described herein. The housing extensions 219 may be fabricated as separate parts which are captured between the sections of the housing or as unitary members of a portion of the housing. The housing portion 207 includes a power switch 260 which is accessible through a flexible portion 260′ of the housing portion 206. The housing shown in FIG. 5F may further include a cable 228 extending from one end of the housing. The cable 228 may be utilized to couple the therapy providing device to another therapy providing device or to a computing device 300 as described herein. It is contemplated that the housing shown in FIG. 5F may not include the cable 228 or the provision for the cable 228, wherein the therapy providing device would utilize wireless communication as described herein.

Referring now to FIG. 5G there is shown an additional embodiment of an alternative housing 210 in accordance with the present disclosure, according to some embodiments. The housing 210 further includes housing extensions 219, wherein the housing extensions 219 maybe integrally formed with the housing 210 or may be embodied as separate components which can be affixed to the housing. It is contemplated that the housing extensions 219 may be permanently attached to the housing 210 or may be detachably attached to the housing 210. As shown in FIG. 5G the alternative housing design includes a power switch 260 and a cable 228 extending from one end of the housing. The cable 228 may be utilized to couple the therapy providing device to another therapy providing device or to a computing device 300 as described herein. It is contemplated that the housing shown in FIG. 5G may not include the cable 228 or the provision for the cable 228, wherein the therapy providing device would utilize wireless communication as described herein.

Referring now to FIG. 5H there is shown an additional embodiment of an alternative housing 210 in accordance with the present disclosure, according to some embodiments. As shown in FIG. 5H the alternative housing design includes a power switch 260 and a cable 228 extending from one end of the housing and housing extensions 219. The cable 228 may be utilized to couple the therapy providing device to another therapy providing device or to a computing device 300 as described herein. It is contemplated that the housing shown in FIG. 5H may not include the cable 228 or the provision for the cable 228, wherein the therapy providing device would utilize wireless communication as described herein.

Referring now to FIG. 5I there is shown an additional embodiment of an alternative housing 210 in accordance with the present disclosure, according to some embodiments. As shown in FIG. 5I the alternative housing design includes a power switch 260 and a cable 228 extending from one end of the housing. The housing shown in FIG. 5I further includes flex portions or hinges 208 formed in the housing extensions 219. The cable 228 may be utilized to couple the therapy providing device to another therapy providing device or to a computing device 300 as described herein. It is contemplated that the housing shown in FIG. 5I may not include the cable 228 or the provision for the cable 228, wherein the therapy providing device would utilize wireless communication as described herein.

Referring now to FIGS. 5J-5N there is shown additional embodiments of an alternative housing 210 in accordance with the present disclosure, according to some embodiments. As shown in FIGS. 5J-5N the alternative housing designs include a power switch 260 and a cable 228 extending from one end of the housing and housing extensions 219, wherein the housing extensions may include fittings 229, wherein the fittings 229 are configured to attach the housing 210 to a bandage as described herein. The fittings can be embodied as magnets 230, snaps, Velcro, metallic inserts or other structures which are capable of forming a removable attachment system. The cable 228 may be utilized to couple the therapy providing device to another therapy providing device or to a computing device 300 as described herein. It is contemplated that the housing shown in FIGS. 5J-5N may not include the cable 228 or the provision for the cable 228, wherein the therapy providing device would utilize wireless communication as described herein.

Referring now to FIGS. 5P and 5Q there is shown an additional embodiment of an alternative housing 1210 in accordance with the present disclosure. The housings shown in FIGS. 5P and 5Q may further include a power switch 260 (not shown) and a cable 228 (not shown) extending from one end of the housing as described herein with regard to other embodiments of the present disclosure. The cable 228 may be utilized to couple the therapy providing device to another therapy providing device or to a computing device 300 as described herein. It is contemplated that the housing shown in FIG. 5P may not include the cable 228 or the provision for the cable 228, wherein the therapy providing device would utilize wireless communication as described herein. Additionally, as shown in FIG. 5P, the alternative design includes an alternative bandage design 6420, wherein the bandage 6420 is formed in the manner described herein. Further still, unlike the other housings disclosed and described herein, the housing 1210 does not include the housing extensions, instead, as shown in FIG. 5Q, the magnets 230 are disposed within the second surface 215 of the housing 1210. It is contemplated that the magnets 230 may be replaced with alternative structures such as snaps, Velcro or other detachable/re-attachable structures.

Referring now to FIGS. 5R-5X there are shown additional embodiments of alternative housings 210 in accordance with the present disclosure. As shown in FIGS. 5R-5X the alternative housing designs include a power switch 260 and a cable 228 (not shown) extending from one end of the housing. The cable 228 may be utilized to couple the therapy providing device to another therapy providing device or to a computing device 300 as described herein. It is contemplated that the housing shown in FIG. 5R-5X may not include the cable 228 or the provision for the cable 228, wherein the therapy providing device would utilize wireless communication as described herein.

Referring now to FIGS. 5Y, 5Z, 5AA, 5BB, and 5CC, there is shown additional embodiments of housings in accordance with the present disclosure. As shown in FIG. 5Y there is shown an additional embodiment the housing 210 in accordance with the present disclosure. As shown in FIG. 5Y the alternative housing design include a power switch 260 which may be configured to transmit light from at least one LED disposed within the housing and a cable 228 extending from one end of the housing. The cable 228 may be utilized to couple the therapy providing device to another therapy providing device or to a computing device 300 as described herein. It is contemplated that the housing shown in FIG. 5Y may not include the cable 228 or the provision for the cable 228, wherein the therapy providing device would utilize wireless communication as described herein.

Referring now to FIG. 5Z there is shown a bottom view of the therapy providing device 200 and housing 210 of FIG. 5Y. As shown in FIG. 5Z, the therapy providing device includes the housing 210, a second surface 215, a driver assembly 220 (embodied as a haptic exciter 220′) and housing extensions 219.

Referring now to FIGS. 5AA, 5BB, and 5CC, there is shown an embodiment of a housing extension 219 in accordance with the present disclosure. As shown in FIG. 5AA-5CC, the housing extension 219 is configured to be received by a portion of the housing 210 and the second surface 215 (not shown), such that the housing extension 219 is retained between the two components. Alternatively, the housing extension 219 maybe integrally formed with either the housing 210 or the second surface 215 of the therapy providing device 200. As shown in FIGS. 5AA-5CC, the housing extension 219 is further configured to receive a fitting 229 within a portion of the arm, wherein the fitting can be a magnet 230 disposed within an optional magnet holder 231. Alternatively, the fitting 229 can be embodied as a snap, VELCRO, a metallic insert, glue or other detachable structures.

The housing extensions 219 in accordance with the present disclosure, according to some embodiments are designed and constructed of a material having a spring force K, wherein the spring force K may be linear or non-linear. In use, the spring force K of the arms in combination with the spring force K2 of the haptic 220′ or speaker voice coil 222 work together to ensure that the coil of the haptic 220′ is never fully compressed during use. A force is initially applied to the voice coil 222 of the haptic 220′, compressing the springs 223 of the haptic 220′ as the force increases, the housing extensions 219 begin to flex thereby relieving the force on the voice coil 222 of the haptic 220′.

Further still, in accordance with the present disclosure, according to some embodiments, the housing extensions 219 of the therapy providing device 200 are housing extensions 219 are designed and may be constructed of a viscoelastic material. When the housing extensions 219 of the housing 210 are attached to the bandage, the extensions of the material will be deformed conforming to the anatomy of the patients collar bone and are designed to continue to conform and adapt to motion of the user and user's collarbones. The deformation of the housing extensions 219 will be highly dependent on the user's anatomy. Some user's might have very pronounced collar bones whereas other user's may have collar bones which are more in plane with their body contour. The housing extensions 219 provide a downward force to keep the voice coil 222 of the haptic speaker 220′ in contact with the user's skin disposed over their collar bone(s). It is preferred that the downward force provided by the housing extensions 219 should not exceed the spring force of the voice coil 222 of the haptic 220′ to avoid compressing the voice coil 222 to much such that the ability of the haptic 220′ to produce vibrations is diminished. The deformation of the housing extension 219 initially provides a downward force, which is configured to decrease over time due to the viscoelastic behavior of the material. The viscoelastic material of the housing extensions 219 is chosen to provide a downward force which will not exceed the compression force of the haptic exciter. Additionally, the material of the housing extensions 219 additionally is chosen such that the force applied to the user does not cause harm to the user. In a preferred embodiment, the viscoelastic material of which the housing extensions 219 provide a downward force between about 0.1 and 6 lbs, preferably between about 0.1 and 4 lbs and most preferably between about 0.25 and 2.5 lbs.

It is also contemplated that in accordance with the present disclosure, according to some embodiments, when a force is applied to the voice coil of the haptic 220′ or speaker, the springs 223 of the haptic 220′ initially compress and the struts 232 of the housing extension(s) 219 are lifted, as the force increases on the voice coil 222 of the haptic, a hysteresis occurs in the housing extensions 219, whereby the housing extensions 219 continue to extend thereby relieving the force on the struts 232/voice coil 222 of the haptic 220′ thereby permitting the voice coil 222 of the haptic 220′ to move independent of the applied force and preventing complete compression of the voice coil 222. Although the above embodiment of the housing arms 219 are described in accordance with a haptic 220′, it is contemplated that the housing arms 219 may be utilized in combination with any driver 220 as described within the present disclosure, according to some embodiments.

In yet another embodiment, it is contemplated that charging pins or charging pads may be disposed in the housing such that they extend beyond the second surface 215 of the housing. In this embodiment, the charging pins/pads may be symmetrically spaced about a centerline axis of the housing 210, such that, when placed onto a charging station, the orientation of the therapy providing device 200 is inconsequential as the charging pins/pads would align with the respective charging pins/pads of the charging station. If pins are utilized in the construction, it is preferred that the pins are spring-loaded retractable pins, such that when the therapy providing device is placed onto a bandage as described below the pins retract within the housing 210. Furthermore, the use of spring loaded pins ensures good electrical contact between the pins and the respective charging pads of the charging station or pads disposed on a bandage as described below with regard to FIG. 16G.

As shown in FIG. 5A, disposed below the first circuit board 500 is an energy source 240. The energy source 240 may be in the form of a battery pack. The battery pack may be a rechargeable pack or a single use pack, which may be embodied as gel batteries or absorbed glass mat batteries. Suitable examples of batteries that may comprise the pack are lithium ion (Li-ion), lead-acid, nickel-cadmium (NiCd), nickel-zinc (NiZn), zinc-oxide, Lithium polymer (LiPo), lead acid, nickel metal hydride (NiMH), Lithium ferrous-oxide (LiFo) or other known battery technologies. It is further contemplated that instead of utilizing a battery for an energy source, a capacitor, capacitor array or a plurality of capacitors and related circuitry could be utilized as an energy source. The energy source may also be disposed above the circuit board, between multiple circuit boards or disposed within the housing in any manner or order or further still disposed outside of the housing and coupled to the circuit board.

In the event that the energy source 240 is embodied as a battery pack, the battery pack may be embodied in the form of a fabricated pack, where individual cells are soldered together, or alternatively, the battery pack could be arranged to utilize conventional battery sizes such as AAAA, AAA, AA, CR2032, LR44, 9-volt, A23 and the like.

It is further contemplated that the battery pack may be further divided into a primary battery pack and a backup battery pack. In use, the primary battery would be initially utilized, if the pack malfunctions or loses its charge or its charge is used, the backup battery pack would then be enabled to continue the therapy.

If the battery pack as described above is chosen to be a rechargeable, there is a need to provide a charging circuit within one of the circuit boards 500 or 550. The charging circuit may utilize either a physical connection to enable charging or may use a non-contact or inductive charging arrangement. If a physical connection is utilized, the plug may be a USB style plug, headphone style, spring loaded pins/contact pads or other types of plugs, such a plug can be integrated into the housing 210 and electrically connected to the battery through either circuit board. Alternatively, a plug may be directly mounted onto one of the circuit boards. It is further contemplated that the charging plug can also be utilized both for charging as well as communication between multiple therapy providing devices 200 and the computing device 300 as described above using a compatible cable. In an embodiment, the charging circuit would include “smart charging” technology, such that the circuitry would be capable of monitoring the charging current, battery temperature, battery life etc. to ensure that the battery is charged optimally as well as being properly charged and not overcharged. Alternatively, the charging station/base may include the “smart charging” circuitry or a portion of the circuitry as well.

As described above, the present disclosure, according to some embodiments may utilize pins or pads disposed on or coupled to the first circuit board to enable charging of the battery disposed within the device. It is further contemplated that a non-contact charging assembly could be utilized with the present disclosure, according to some embodiments. If a non-contact charging arrangement is selected, then the charging pins 265 and/or openings within the first surface of the housing 210 may not be necessary. Instead, the therapy providing device 200 would include a charging coil (not shown) disposed about the perimeter of the first circuit board 500. The use of a non-contact charging coil would further necessitate the inclusion of additional integrated circuits to enable and control the charging function. These additional circuits can be disposed on either of the two circuit boards. Suitable examples of a non-conductive or inductive charging would utilize an electromagnetic field to transfer energy between the charger and the battery pack. In this embodiment a charging station would be provided in which the therapy providing device 200 could be stored and charged simultaneously as will be described below. It is also contemplated that the storage/charging container may be a smart container that is it may contain a microprocessor and/or a wireless communication chipset. Thus, once the therapy device is removed from the storage container, the integrated wireless chipset within the storage container may cause the therapy device to power on. Suitable examples of components to enable non-contact charging are available from Wurth Electronics Inc., part numbers 760308201 wireless charging receiving coil and 760308101 wireless charging transmitting coil.

In accordance with the present disclosure, according to some embodiments, it is contemplated that the energy source 240 may be embodied in the form of an integrated generator, wherein the generator would be configured to create energy from movement of the therapy providing device 200, much like and automatic watch movement.

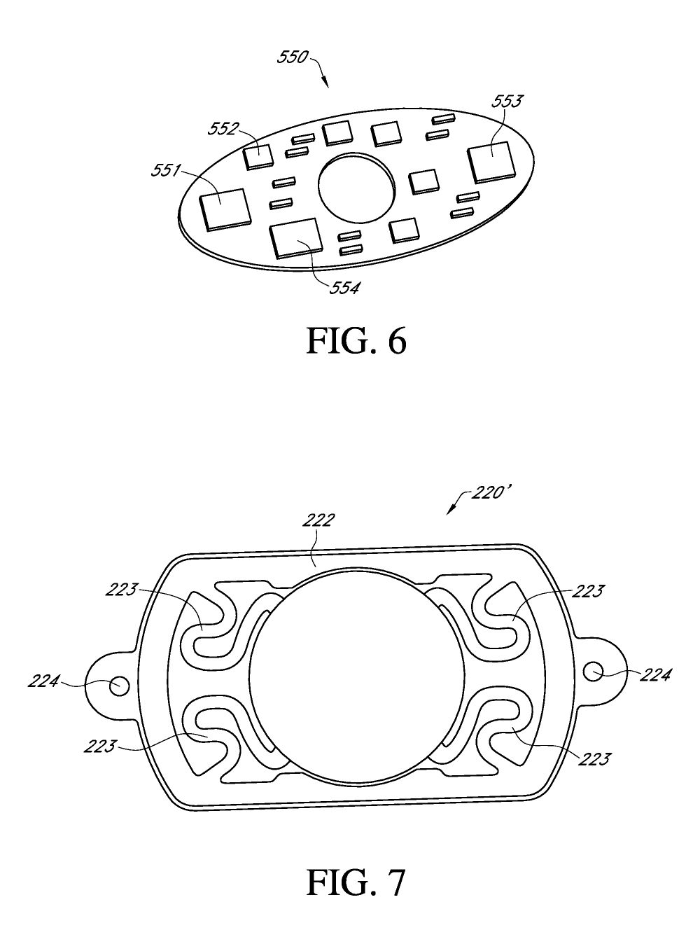

As described above, the therapy providing device 200 shown in FIG. 5 includes two circuit boards, 500 and 550. The circuit board 500 having been previously described above. Referring now to FIGS. 5 and 6 there are shown exemplary embodiments of the circuit board 550 in accordance with the therapy providing device 200 of the present disclosure. As described above, the second circuit board 550 is configured to be coupled with the first circuit board 500, wherein components may be disposed on either of the two boards and interconnected through an appropriate connection as previously described using a header or solder holes formed in the circuit board 550. Referring now to FIG. 6, there is shown a general schematic of the second circuit board 550, wherein the second circuit board 550 includes a processor 551, optional memory chip 552, an audio amplification circuit 553 and a communication port 554. The communication port 554 may embodied as a physical port such as a mini-usb, micro-usb, firewire, thunderbolt or other known similar communication ports. The audio amplification circuit 553 may include one, two, three, four, five, six, ten, twelve, fourteen, sixteen audio amplifiers, wherein the incoming signal from the processor 551 is amplified such that the amplified signal can then be connected to a driver assembly 220 as described below. As shown in FIGS. 5 and 6, the second circuit board 550 may be shaped to be received within a shaped housing. As shown in FIGS. 5 and 6, the second circuit board is shown having an elliptical shape with an aperture formed through the center thereof. The aperture can be sized to receive a portion of the driver assembly 220, thereby allowing the overall size of the device to be reduced by allowing components to ‘nest’ when assembled. The second circuit board 550 may contain additional electronic components such as audio filters, booster circuits, timing circuit and data logging capability. In accordance with the present disclosure, according to some embodiments, the processor 551 may be sourced from CSR PLC, Churchill House, Cambridge Business Park, Cowley Road, Cambridge, CB4 0WZ Churchill House, Cambridge Business Park, Cowley Road, Cambridge, CB4 0WZ, having part number 8670. It is contemplated that other processors can be utilized with the present disclosure, according to some embodiments and the processor described above should be considered exemplary.

The second circuit board 550 may further include a communications chipset (555 not shown) such: BLUETOOTH, WIFI, ZIGBEE, RFID, NFC, Ant+, infrared, 3G/4G, CDMA, TDMA or other known wireless communication protocols.

The first or second circuit board 500/550 may further include a clock circuit (556, not shown). The clock circuit generates and sets the timing of operations performing within the therapy providing device 200. The clock generator may be utilized to activate the therapy providing device 200, or may be utilized to record timed events, such as when the therapy providing device is on or off or in use.

Further still, either circuit board 500/550 may alternatively include an impedance sensor (557, not shown) or pair of impedance sensors, the impedance sensors in association with the processor 551 can be used to determine if the housing 210 and/or therapy providing device 200 is coupled to a user's skin or to a bandage 420 or if the housing is not coupled to the skin or bandage 420. If the housing 210 is coupled to a user's skin, then the impedance sensor would provide a signal to the processor 551 indicating such a condition, thereby the program stored in the memory of the processor or within memory or transmitted to the microprocessor could be initiated to conduct therapy according to the disclosure. If the impedance sensor is not coupled to the user's skin, then an open condition would occur, whereby the program would not be initiated and a visual signal may be generated through the program/processor to alert the user that the therapy providing device 200 is not placed properly and needs to be repositioned. As described below, the impedance sensors may be utilized in combination with features of the bandage to determine when the therapy providing device is coupled to a bandage, as such, the therapy providing device may then be energized, manually or automatically or in response to a program command as desired.

In yet another embodiment, the electronics module may include a microphone (558, not shown), whereby a test signal can be initiated and delivered by the driver assembly 220 or other audio/vibration device. The microphone would be utilized by the processor 551 to listen for a reflection of the test signal off of the user's clavicle, skin or other bone or structure to determine if the therapy providing device 200 has been placed properly. If the reflected sound matches that of one stored in memory, then the program can be run to provide therapy. If the reflected sound does not match the sound stored in memory, then an error message would be generated. The error message may be in the form of an audio signal or in the form of a visual signal such as a blinking light or a series of blinking lights. The microphone can also be utilized to record a user's breath sounds or sounds emanating from the user's thorax. This may be utilized to determine if the user snores during sleeping or may be suffering from sleep apnea. The recorded sounds could then be wired/wirelessly transmitted to enable diagnosis of sleep apnea.

Additionally, the microphone 558 could be coupled with a blood pressure monitor, wherein the microphone 558 would listen for Korotkoff sounds, whereby the data generated from the blood pressure monitor and specifically the Korotkoff sounds captured by the microphone can be utilized to enable a closed loop control system or closed loop feedback system. It is contemplated, that the therapy provided by the therapy providing device 200 can be dynamically modified in response to the data received from the microphone coupled to the processor 551.

The circuit board 500 or 550 may further incorporate a pressure sensitive switch coupled to the processor 551. In use, the pressure sensitive switch would be in a normally open position or off position. When the therapy providing device 200 is placed on the user's skin, the pressure sensitive switch would be depressed, thereby turning the therapy providing device 200 on. The actuation of the switch can also be associated with the clock circuit to associate a time with the on/off state of the switch. These events can be written to the memory of the processor 551 or other memory storage location. The data can then be transmitted, wired or wirelessly, to a personal computer for analysis/storage. By tracking the actual on/off time of the therapy providing device, user compliance may be tracked by the user or by a third party such as a health care provider.

In yet another embodiment, the circuit board 500 or 550 may include an optical sensor, 559 (not shown) wherein the optical sensor 559 is utilized to detect whether the therapy providing device is affixed to a user's skin. In this embodiment, the optical sensor 559 can include a light sensor 560, whereby when the therapy providing device 200 is affixed to the user's skin the light is blocked to the sensor. In another embodiment, the optical sensor can be a reflective sensor 561, wherein the color of the light reflected back indicates whether the device is affixed to a user's skin or not. Further still, the optical sensor may be utilized to indicate if the therapy providing device is coupled to a bandage, whereby the therapy providing device may be prevented from being activated unless the optical sensor detects the presence of a bandage or a reflective sensor disposed on the bandage.

In another aspect of the present disclosure, according to some embodiments, the light sensor may be utilized to monitoring blood oxygen level, wherein data received from monitoring the user's blood oxygen level can be stored in memory or transmitted to another device such as a pulse-oximetry monitor or another computing device. Further still, the blood oxygen data may be utilized by a program of the therapy providing device to alter therapy provided to the user or otherwise control the therapy providing device 200. The light sensor may also be configured to respond to ambient light conditions. For example, the sensor may be configured to initiate the therapy providing device 200 when the sensor senses darkness or a programmed threshold of darkness and turn off the therapy providing device when sensing lightness.

In another aspect of the present disclosure, according to some embodiments, the light sensor may be used to measure alteration in blood-reflectance color, whereby the program controlling the therapy providing device may utilize this signal as a representation of heart rate or heartbeat. Accordingly the program controlling the therapy providing device 200 may use this data to determine blood pressure and accordingly provide therapy to the user based on the received data.

In another aspect of the disclosure, the therapy providing system 100 may be activated by disposing the two therapy providing devices 200 adjacent to each other. In this embodiment, magnets 340 disposed within the housings 210 may be utilized to activate a magnetic switch disposed within the housing 210 of each therapy providing device. In another aspect, one of the therapy providing devices may include one component of a RFID reader and the other therapy providing device includes the second component of the RFID system, such that when brought within proximity of each other the therapy providing devices may become activated. Alternatively, a user could use a wristband or a card containing a RFID component, such that when the wristband/card is brought into proximity with the therapy providing device containing a corresponding RFID circuit, the therapy providing device would be turned on. In another aspect, one therapy providing device may include an optical scanner and the other therapy providing device includes a barcode, QR code or other optically readable target to which the optical scanner would respond to.

It is further contemplated, that an accelerometer 670 (not shown) and/or compass 671 (not shown) and/or tilt sensor 672 (not shown) and/or GPS sensor 673 (not shown) can be incorporated into either of the circuit boards described above. The inclusion of such a sensor can be utilized to determine the position and/or orientation of the device. Additionally, the inclusion of such sensors along with a computer readable program can be utilized to track or monitor a user's sleep and sleep pattern. The user can then review this data on either the therapy providing device through the inclusion of a display 250 (not shown) or series of LED lights incorporated into the housing 210 of the device. Alternatively, the sleep data may be transmitted to the computing device 300 or transmitted to a health care provider. Further still, the sleep data may be displayed on the charging base when the therapy providing device(s) 200 are coupled to the charging base. In use, as described below, a user would affix the housing 210 to their person using an adhesive patch, harness, specialized clothing article as will be described below. In this embodiment, the accelerometer/compass/tilt/GPS sensor is in communication with the processor 551 can be utilized to determine when to activate the therapy providing device or devices 200. If the signal coming back from the accelerometer/compass/tilt sensor indicates that a therapy providing device 200 is in a vertical position, then the program contained within the memory of the processor 551 or computing device 300 would not be initiated. Once the signal from the accelerometer/compass/tilt/GPS sensor indicates that the user is in a prone position, likely a sleep position, then the program contained within the memory can be run. Additionally, the clock timer can be associated with the accelerometer/compass/tilt/GPS sensor such that a user's sleep pattern can be stored in memory of the processor 551 or computing device 300. Data generated from such sensors could be stored in memory, of either the therapy providing device or the computing device to track usage of the device as well as the physical location of the devices. Such data could be transmitted to a third party using know wireless communication methods.

As described above, the therapy providing device 200 of some embodiments of the present invention may include a display 250 (not shown), the display 250 may be integrated into the housing 210 or visible through the housing 210. The display 250 may comprises a series of LEDs, which may be RGB LEDs such that specific colors and/or numbers of illuminated LEDs could be utilized to convey information to a user. Alternatively, the display 250 may be embodied as a LCD, VFD, LED or OLED or other similar type displays wherein text and/or characters/pictures can be visually displayed on the display 250. In use, the display can be utilized to display information such as time, battery capacity/reserve, therapy start/stop time, therapy duration, sleep duration, movement during sleep, respiration rate, room temperature, body temperature, blood oxygen level, heart rate or be used to display data collected by a third party device such as a scale, fitness tracker, sleep apnea device. The display can be further utilized to display a reminder to the user to re-charge the therapy providing device 200 after a therapy session. It is also contemplated that the characters shown on the display may be displayed in a backwards fashion, thereby allowing a user to view the display while standing in front of a mirror while wearing the device. The display may be programmed to switch between displaying the data backwards and conventionally.

The circuit board(s) 500/550, program, the housing 210 or therapy providing device 200 may be provided with a unique identifier such as a serial number or patient information identifier so that the therapy providing device 200 may be tracked. Additionally, using the unique identifier it may be possible for a physician or a user to utilize a computer program, such as a website which when placed in communication with the therapy providing device, either wired or wirelessly, would allow continuous monitoring of usage of the device, such as date and time monitoring, duration of use, patient compliance and the like. The website could also provide information regarding hypertension and additionally be configured to communicate with other devices such as a scale to track the user's weight, a blood pressure monitor to track blood pressure measurements, a glucose meter, a heart rate monitor or other fitness tracking device such as FITBIT, JAWBONE UP, or BODYBUG. Each of these devices would be interfaced with the website such that data collected from these devices could be uploaded to the website where the data could be presented to the user or alternatively, the data could be shared with anyone that the user chooses to do so. For example, the user may desire to share the data with their health care provider, dietician or other individual(s). Further still, the user may choose to interface the data with a social media site such as Facebook. Additionally, if the charging base 570 includes a pill container, then information regarding a user's medication compliance data could also be shared on the web site.

In accordance with the disclosure, it is contemplated that one or both circuit boards along with the battery may be housed within a separate housing from the therapy providing device 200. In this embodiment, the circuit board(s) and battery would be coupled to the therapy providing device either through a cable connection or through a wireless connection. If a wireless connection is utilized, then the therapy providing device would include the necessary electronics disposed within its housing to facilitate the communication between the electronics module and the therapy providing device as well as a power source such as the battery.

Referring to FIG. 5A, disposed below the second circuit board 550 is a driver assembly 220. The driver assembly 220 is at least partially disposed within the volume 213 of the housing 210. In some embodiments, the voice coil 222 may extend beyond the second surface 217 of the housing 210. The driver assembly may comprise a conventional coil speaker, an ultrasonic generator, a piezoelectric speaker, a haptic speaker, a pneumatic device, a suction device, a mechanical vibratory device, a hydraulic actuation device, planar ribbon speaker, electrostatic speaker or a photo-acoustic excitation device. Examples of drivers assemblies 220 that can be used with the present disclosure, according to some embodiments may be purchased from HiWave Technologies PLC, Regus House, 1010 Cambourne Business Park, Cambourne, Cambridge CB23 6DP United Kingdom. Referring now to FIG. 7 there is shown an exemplary haptic speaker or haptic exciter 220′ which may be utilized with the therapy providing device 200 of the present disclosure, according to some embodiments. As shown in FIG. 7, the haptic speaker 220′ includes a frame member 221, a voice coil 222 and a plurality of flexible members 223. Additionally, the haptic speaker 220′ includes electrical connections 224, thereby allowing the haptic speaker 220′ to be electrically connected to the audio amplifier circuit as previously described. In use, the flexible members 223 allow the voice coil 222 of the haptic speaker 220′ to translate relative to the frame 221, thereby producing sound or movement.

The driver assembly includes a portion which is configured to extend beyond the housing. The portion of the driver assembly which extends beyond the housing is configured to engage a user, a user's tissue or a user's skeletal frame, particularly the clavicle. The portion may extend beyond the housing between 0 mm and 1 mm, 0 mm and 1.5 mm, 0 mm and 1.75 mm, 0 mm and 2 mm, 0 mm and 2.1 mm, 0 mm and 2.2 mm, 0 mm and 2.3 mm 0 mm and 2.4 mm, 0 mm and 2.5 mm, 0 mm and 2.6 mm, 0 mm and 2.7 mm, 0 mm and 2.8 mm, 0 mm and 2.9 mm, 0 mm and 3.0 mm, 0 mm and 3.1 mm, 0 mm and 3.2 mm, 0 mm and 3.3 mm, 0 mm and 3.4 mm, 0 mm and 3.5 mm, 0 mm and 3.6 mm, 0 mm and 3.7 mm, 0 mm and 3.8 mm, 0 mm and 3.9 mm, 0 mm and 4.0 mm, 0 mm and 4.1 mm, 0 mm and 4.2 mm, 0 mm and 4.3 mm, 0 mm and 4.4 mm, 0 mm and 4.5 mm, 0 mm and 4.6 mm, 0 mm and 4.7 mm, 0 mm and 4.8 mm, 0 mm and 4.9 mm, 0 mm and 5 mm, 0 mm and 5.5 mm, 0 mm and 6 mm, 0 mm and 7 mm, 0 mm and 8 mm, 0 mm and 9 mm, 0 mm and 10 mm, 0 mm and 11 mm, 0 mm and 12 mm, 0 mm and 13 mm, 0 mm and 14 mm, 0 mm and 15 mm, 0 mm and 16 mm, 0 mm and 17 mm 0 mm and 18 mm, 0 mm and 19 mm and 0 mm and 20 mm, 0 mm and 30 mm, 0 mm and 40 mm, 0 mm and 50 mm. In a preferred embodiment the portion of the driver assembly extending beyond the housing is between about 0 mm and 5 mm and more preferably between 0 mm and 4 mm and most preferable between 0 mm and 3 mm.

It is further contemplated that the portion of the driver assembly which is configured to extend beyond the housing may be constructed of multiple pieces. In accordance with the disclosure, the projecting portion of the driver assembly may include a first portion affixed to the driver assembly and second portion, wherein the second portion is threadably attached to the first portion. The projection distance of the driver assembly beyond the housing can thereby by adjusted by screwing the second portion in/out of the first portion. In another example, the portion of the driver assembly projecting beyond the housing may comprise a first portion fixed to the driver assembly and a second portion, which is removable. The distance of the projection can be adjusted by changing the second portion, such that the second portion can be manufactured having multiple different heights. Lastly, it is contemplated that the housing may be configured such that the entire drive assembly can be moved within the housing, thereby allowing for adjustment of the height of the projecting portion of the driver assembly outside of the housing.

In yet another embodiment, the driver assembly 220 may be embodied as an electroactive polymer transducer 315 as shown in FIGS. 8-10. Electroactive polymer transducers are made up of a first thin elastic polymer 320, which is also referred to as a film or membrane, this is sandwiched between compliant electrodes 340 and 345. When voltage is applied across the electrodes, the unlike charges in the two electrodes are attracted to each other, these electrostatic attractive forces compress the polymer film 320 (along the z-axis). The repulsive forces between like charges in each electrode stretch the film in the plane (along the X and Y axis′). As the transducer 315 deflects, the deflection can be utilized to perform work. In the present disclosure, according to some embodiments, the work that is performed is the development of vibrations, wherein the vibrations being developed by the transducer 315 are developed within a certain frequency range as will be discussed in greater detail below. Additional information regarding electrostatic transducers can be found in U.S. Pat. No. 7,898,159 and U.S. Pat. No. 7,608,989, the entireties of which are hereby incorporated by reference.

It is further contemplated that the transducer 315 as described above may be further coupled to another assembly, wherein the other assembly would have an increased mass. Through use, the transducer would be activated by providing a voltage to the electrodes, thereby exciting the polymer, wherein the weighted assembly would be excited thereby delivering greater vibrational energy.

In accordance with another aspect of the present disclosure, according to some embodiments, the electroactive polymer transducer 315 can be formed to have a curved shape, or be attached to a housing having a curved shape, such that the housing or curved excited can be readily received by a user's anatomy, specifically the user's clavicle or collarbone.

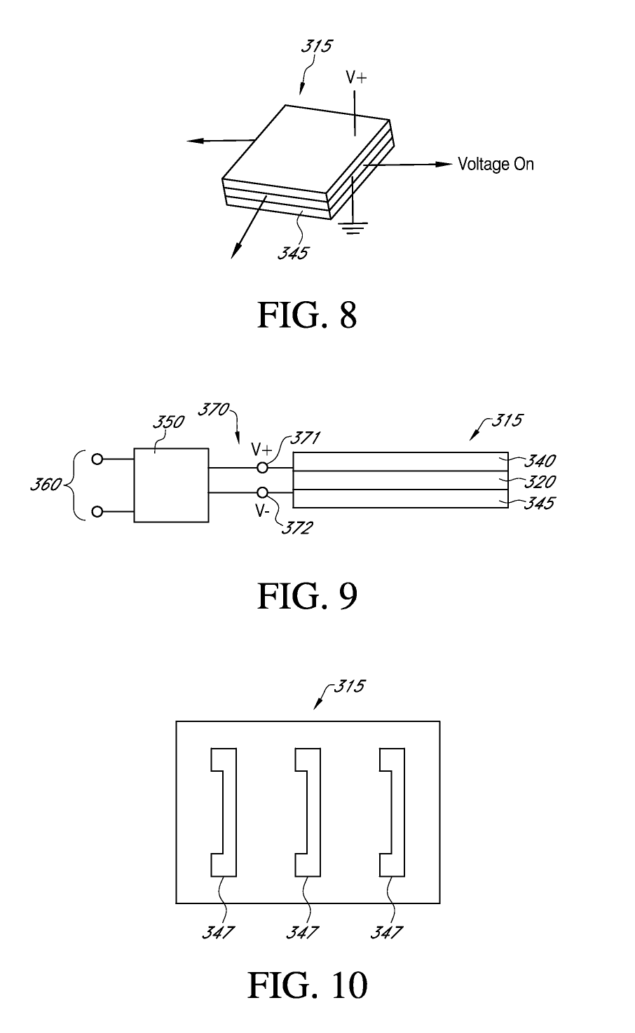

The electroactive polymer transducer 315 of the present disclosure, according to some embodiments may be embodied in different geometric shapes. It is contemplated that the transducer 315 may be embodied in the form a circular shape, oblong shape, square, rectangular or other known geometric shapes. Further still, it is contemplated that the transducer may be formed with at least one bar-arm type of arrangement as shown in FIG. 10. In this embodiment, the bar-arm 347 is configured to vibrate in response to the charge placed on the electrodes. The number of bars and shape of the bars can be configured to adjust the acoustic/vibrational properties of the assembly.

Use of an electroactive polymer transducer as described above further includes a circuit driver 350, the circuit driver 350 may be incorporated into the first or second circuit boards 500/550 as described above. Alternatively, the circuit driver 350 as shown in FIG. 9 may be embodied as a separate circuit board (not shown) which may be electrically coupled with either the first or second circuit boards of the present disclosure, according to some embodiments. The circuit driver 350 further includes an audio input 360 and at least one output 370, but preferably a pair of outputs 371 and 372. The outputs 371, 372 are coupled to the electrodes 340, 345 of the transducer 315.

The circuit driver 350, may further include additional components such as an amplifier, a filter, a voltage step-up circuit, a charge controller, voltage step-down.

Further still it is contemplated that the driver assembly may be embodied as multiple elements, for example any combination of driver assemblies may be use, such as a combination of a haptic speaker and a piezo, a haptic speaker and an electro active polymer transducer, an electroactive polymer transducer and a piezo or multiples of the same driver type within the same housing. The examples provided herein should not be considered limiting in any manner. Alternatively, the driver assembly may be a vibrating motor or coin cell motor.

As described above and in accordance with the present disclosure, according to some embodiments, it is contemplated that two therapy providing devices 200 may be utilized together to provide therapy to a user, wherein the two therapy units may be interconnected with a physical connection. It is contemplated that one of the therapy devices may have a complete set of electronics disposed therein, wherein the complete set of electronics would include the communication, memory and other chipset(s) and associated circuitry. Wherein the other therapy providing module 200 could then include a simplified electronics module, wherein the simplified electronics module would not have the complete chipset of the complete electronics module. For example, the simplified electronics module would not need to have a battery charging circuit or other chips as well it may have less or no memory. By providing the other therapy providing device with a slimmed down electronics module a larger energy source may be fitted, through this arrangement the combined therapy providing devices 200 could be utilized for a longer time before the energy source would need to be replaced or recharged.

Further still, in an embodiment, the system may comprise at least two therapy providing devices 200, each therapy providing device having a housing 210. Wherein each of the housings includes a driver assembly, and one of the housings includes at least one circuit board and the other housing includes the energy source. The two housings are then coupled together through a cable connection. The cable may be permanently attached to each of the housings or may be detachably attached, wherein if detachably attached, locking connectors on the ends of the cables may be utilized to secure the cable ends to each of the therapy providing devices. By splitting the components between the two separate housings this allows for smaller sized housings. It is further contemplated that the cable connecting the two therapy providing devices may further include additional features such as a power switch, charging port or light emitting diodes to indicate device status. Additionally, the housing including the circuit board may additionally include a power source.

In another embodiment, it is contemplated that certain components of the therapy providing system as disclosed herein may be divided up into multiple housings. In an embodiment according to the present disclosure, according to some embodiments, the driver assembly 220 is disposed within a housing 210′ and the circuit board 500/550, energy source 240 are disposed in a second housing 210″. In this embodiment, two driver assemblies are configured to be coupled to the electronics in the second housing 210″. The driver assemblies may be coupled through a wired connection or through the use of a removable wired connection. For a removable wired connection, at least one wire extends from the housing 210″, the other end of the wire includes a connector, such as a snap connector, a magnetic power connector or magnetic connector (similar to APPLE's mag-safe power connector). The housing 210′ includes a similar connector as to the one on the end of the wire. In use, the user can dispose the housings 210′ onto their body as described herein in relation to the therapy providing system 100. The housing 210″ may be disposed onto a user's skin in a similar manner as described herein or alternatively, the user may wear a special clothing article, such as a pajama top, shirt, harness or other article capable of retaining the housing 210″. After disposing the housing 210″ onto their body, the user would then connect the free end of the wire to each housing 210′.

Referring now to FIG. 11 there is shown a charging/base station 570 in accordance with the present disclosure, according to some embodiments. As shown in FIG. 11, the base station 570 includes a housing 571, wherein the housing 571 includes recessed portions 572 configured to receive the therapy providing device 200 therein. The recessed portions 572 are configured to include charging pins 574, which when the therapy providing device 200 is disposed within the recess will align with the charging pins/pads 265 of the therapy providing device. In addition to the charging pins 574, other pins may be included both on the base 570 and the therapy providing device 200 which may be used for other purposes such as downloading data stored within memory of the therapy providing device(s) 200. The base station 570 may further include a wired or wireless connection to the internet or other network such that the data received from the therapy providing device(s) can be transmitted or uploaded to a webpage as described above or transmitted to another location such as a health care provider or other location for storage. It is further contemplated that the base 570 may include additional features such as an alarm clock or clock 573, a cellular telephone or tablet charging station. If an alarm clock is included in the base 570, the alarm could be configured to alert a user to return the therapy providing device 200 to the base 570 for charging after a therapy session. As will be described in greater detail below and herein with regard to FIGS. 5A-5Z, the therapy providing device may include extensions 219 extending from the housing 210 each extension 219 containing a magnet 230. It is contemplated that the magnets 230 may be disposed within a pocket or aperture of the second surface 215 or that the magnets/metallic members may be disposed within either of the housing portions during a manufacturing process such as molding or injection molding. It is contemplated that the recessed portions of the charging base 570 may be shaped to receive the extensions 219 of the housing 210 shown in FIG. 11. The recessed portions 572 may be adapted to receive the extensions 219 or may further include a metallic member or a magnet disposed therein, such that when the therapy providing device is placed into the recessed portion, the magnets 230 within the housing 210 of the therapy providing device 200 are attracted to the metal or magnet of the charging station 570, thus, temporarily affixing the therapy providing device 200 to the charging station. In addition to temporarily affixing the therapy providing device 200 to the charging station 570, by temporarily affixing the therapy providing device 200 to the charging/base station 570 providing better contact between the therapy providing device 200 and the charging pins 574. It is contemplated that other arrangements to increase contact between the therapy providing device and the charging pins of the charging/base station may be utilized. For example, the charging pins 574 in the base station 570 may be configured to move linearly and be held with a spring force, whereby the charging pins 574 retract or partially retract when a therapy providing device 200 is placed into the recessed portion 572 for charging. Additionally, another member (not shown), such as a plate or weights may be placed onto the therapy providing devices after the therapy providing devices have been disposed within the recessed portions. Further still, the charging pins 574 may be disposed on a lid (not shown) of the charging base 570, such that the therapy providing device 200 is placed within a recessed portion 572 of the charging base 570 and the lid is closed, thereby completing the electrical connection between the charging pins 574 of the charging base and the charging pins/pads 256 of the therapy providing device 200.

In accordance with the disclosure, the charging base 570 may be further embodied as another medical device or may incorporate other medical devices. It is contemplated that the base 570 may incorporate, or be incorporated into another medical device such as a pulse-oximetry meter, a blood pressure monitoring device, such as a wireless blood pressure cuff, a glucose meter, an infusion pump, a glucose pump, sleep tracking device, temperature measuring device, or a sleep apnea device such as those offered by RESMED and RESPIRONICS, a thermostat such as the NEST, an internet access point. Presently, sleep apnea devices utilize a console which houses the electronics necessary to control a blower to deliver pressurized air to a patient interface. The patient interface may be embodied in the form of a full-face mask, nasal mask, oro-nasal mask, mouth mask, nasal prongs, or other suitable configurations know in the art. Also, any suitable headgear arrangements may be utilized to comfortably support the patient interface in a desired position.

It is further contemplated that the charging base 570 may further include a medicine dispenser. For example, the charging base 570 may include a pill storage container 575 (not shown) in which the user can place medications which they take daily, the charging base 570 or an alarm clock of the charging base 570 may be programmable to remind a user to take their medication on a daily basis, wherein such reminders can be embodied as a visual, audible or a visual/audio alarm. The pill storage container 575 could include a weight sensor, optical sensor and/or ultrasonic sensor to determine if there are contents (pills/medication) within the storage container. The sensor can then be utilized to trigger the alarm or text if the user has not taken their medication. The alarm/text could be programmed to continue until the contents of the container are emptied. Alternatively, the reminder may be a text message or other electronic message. Additionally, the charging base can also monitor the compliance of the user, i.e., whether the user takes her medications or not and/or whether she charges/removes the therapy providing device from the charging base 570.

The charging base 570 can also be configured to serve as a communications portal for other wireless health devices, such as: scales, fitness trackers, sleep sensors, blood pressure measuring devices, glucose meters, motion tracking devices, personal emergency response pendants, pulse/heart rate monitors, pulse-oximetry monitors, and the like. The charging base 570 can also be configured to be a connection “hub” for other devices which may be located in a user's home, for example, the charging base 570 may include features such as a thermostat or thermostat interface which allows a user to control their homes heating/cooling system from their bedside. The base 570 includes the necessary circuit board(s) and chipsets to enable communication of the devices mentioned above, as well as providing connectivity to the internet to allow remote management of the devices listed above as well as the therapy providing device.

In yet another aspect of the present disclosure, according to some embodiments, referring now to FIG. 12 there is shown the therapy providing device 200 of the present disclosure, according to some embodiments, wherein the therapy providing device 200 has been adapted to interface with a sleep apnea patient interface. As show in FIG. 12, the therapy providing device 200 is configured to be received or engage or is integrated into the headgear arrangement of a sleep apnea patient interface device, wherein the therapy providing device of the present disclosure, according to some embodiments is configured to engage the patient's jawbone or skull.

In yet another aspect of the present disclosure, according to some embodiments, the therapy providing device 200 may be incorporated into other devices which are configured to engage a patient's tissue and skeletal bones such as bone conduction hearing aids, one such example is being offered by Sonitus Medical under the tradename SOUNDBITE. Other such examples of devices into which the therapy providing device 200 of the present disclosure, according to some embodiments may be incorporated into also includes bone conducting headsets, such as those being developed and sold under the name AFTERSHOKZ and by PANASONIC. In this embodiment, the driver assembly and associated components, such as the electronics and energy source can be combined with the bone conducting headset assembly.

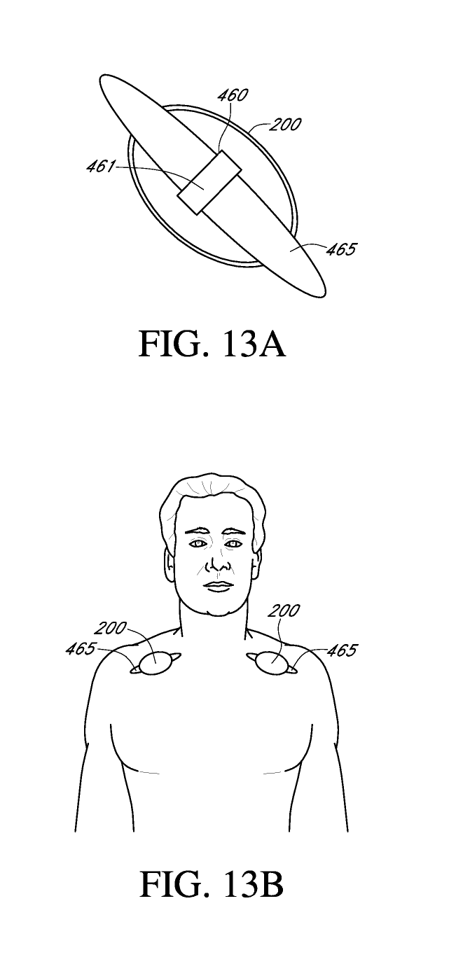

Referring now to FIGS. 13A, 14A, 14B, 15A, 15B, 16A, 16B, 16C, 16D and 16E there are shown multiple embodiments of the housing 210 and bandage assembly 400, 420 and 465 of the therapy providing device 200 in accordance with the present disclosure, according to some embodiments. As shown in these figures, the housing 210 is shown having a variety of mounting assemblies that can be utilized to affix the therapy providing device 200 to the patient.

As shown in FIG. 13A, one surface of the therapy providing device is provided with a slot 460. A bandage 465 can be passed through the slot 460, wherein a rib 461 formed by slot 460 retains the therapy providing device 200 onto the bandage 465. The bandage 465 further includes a biocompatible adhesive, such that the therapy providing device 200 can be affixed to the patient as shown in FIG. 13B. The bandage may be a one-time use construction, wherein the bandage is disposed of after a single use. Alternatively, the bandage 465 may be a multiple-use product, wherein the biocompatible adhesive is selected such that the bandage can be placed and removed from a user's skin multiple times. Additionally, it is contemplated that the biocompatible adhesive may be renewed. The adhesive may be renewed by spreading new adhesive over the existing adhesive, washing the adhesive surface with a substance to renew the surface or the adhesive may be embodied having multiple thin layers, wherein the user removes the used layers and disposes of the used layer, thereby exposing a new layer of adhesive for use again. A suitable example of an adhesive for a reusable bandage are hydrogel adhesives, similar to those utilized on electrodes for electrical muscle stimulation devices, otherwise known as a TENS unit. Such electrodes are manufactured and sold by 3M as well as others. It is contemplated, that a temporary marking may be applied to the user's body initially to indicate the location of where the therapy providing device. For example, the temporary marking may be in the form of a temporary tattoo or a henna tattoo. Other suitable adhesives include silicone adhesives, hydrocolloid adhesives and acrylic adhesives. In a preferred embodiment a silicone adhesive is utilized.

In yet another embodiment, the thickness of the adhesive may be chosen so that a portion of the driver assembly couples with the adhesive to better enable the driver assembly to transmit vibrations to the user. Additionally, it is contemplated that over a period of time, the motion of the driver assembly may cause the adhesive to creep or otherwise become displaced such that a portion of the driver assembly becomes embedded within or surrounded by the adhesive

In yet another embodiment it is contemplated that the bandage of the present disclosure, according to some embodiments may be configured as a double sided adhesive member, where in the user would apply the double sided adhesive over the collar bone as described herein, they would then remove the backing off of the second side of the adhesive thereby exposing the adhesive. The therapy providing device 200 may be configured to then be directly adhered to the second side of the adhesive on the user's skin.

In another embodiment of the present disclosure, it is contemplated that the user may first apply an adhesive to their skin in the area of the collar bone. They would then apply either the therapy providing device 200 directly to this adhesive or apply a bandage onto the adhesive, wherein the bandage includes a fitting configured to engage a fitting of the therapy providing device as described herein.

It is further contemplated that the bandage may be left in place on the user's skin for a period of time of continuous wear, for example, 1 minute, 2 minutes, 3 minutes, 4 minutes, 5 minutes, 10 minutes, 12 minutes, 15 minutes, 20 minutes, 25 minutes, 30 minutes, 35 minutes, 40 minutes, 45 minutes, 50 minutes, 55 minutes, 1 hour, 1.25 hours, 1.5 hours, 1.75 hours, 2 hours, 2.25 hours, 2.5 hours, 2.75 hours, 3 hours, 3.25 hours, 3.5 hours, 3.75 hours, 4 hours, 5 hours, 5.25 hours, 5.5 hours, 5.75 hours, 6 hours, 6.25 hours, 6.5 hours, 6.75 hours, 7 hours, 7.25 hours, 7.5 hours, 7.75 hours, 8 hours, 8.25 hours, 8.5 hours, 8.75 hours, 9 hours, 9.25 hours, 9.5 hours, 9.75 hours, 10 hours, 10.25 hours, 10.5 hours, 10.75 hours, 11 hours, 11.25 hours, 11.5 hours, 11.75 hours, 12 hours, 12.25 hours, 12.5 hours, 12.75 hours, 13 hours, 13.25 hours, 13.5 hours, 13.75 hours, 14 hours, 14.25 hours, 14.5 hours, 14.75 hours, 15 hours, 15.25 hours, 15.5 hours, 15.75 hours, 16 hours, 16.25 hours, 16.5 hours, 16.75 hours, 17 hours, 17.25 hours, 17.5 hours, 18 hours, 18.25 hours, 18.5 hours, 18.75 hours, 19 hours, 19.25 hours, 19.5 hours, 19.75 hours, 20 hours, 20.25 hours, 20.50 hours, 20.75 hours, 21 hours, 21.25 hours, 21.5 hours, 21.75 hours, 22 hours, 22.25 hours, 22.5 hours, 22.75 hours, 23 hours, 23.25 hours, 23.75 hours, 1 day, 1.5 days, 2 days, 2.5 days, 3 days, 3.5 days, 4 days, 4.5 days, 5 days, 5.5 days, 6 days, 6.5 days, 7 days, 7.5 days, 1 week, 1.5 weeks, 2 weeks, 2.5 weeks, 3 weeks 3.5 weeks or longer. If the bandage is left in place, the bandage is constructed to be capable of being exposed to water, soap and the like when the user showers.

Alternatively, a bandage large enough to cover the entire housing of the therapy providing device 200 may be utilized. In this embodiment, the bandage would hang over the edge of the housing by a sufficient amount, such that when the therapy providing device 200 is placed against the tissue of the user, the bandage could be affixed to the tissue to hold the therapy providing device in a desired position. In this embodiment, the bandage may include an aperture, an opaque section or otherwise transparent section, such that when the bandage is placed over the therapy providing device 200, the button 260, LEDs 262 and charging pins/ports 265 on the top surface of the housing 210 of the therapy providing device 200 described above are visible and accessible if the housing includes such components. Such as bandage maybe constructed to further include a one-way membrane, wherein moisture under the bandage may be transported or migrate from the tissue surface through the bandage, however, the bandage would not allow fluid to pass from the outside to the therapy providing device 200 or the user's tissue.

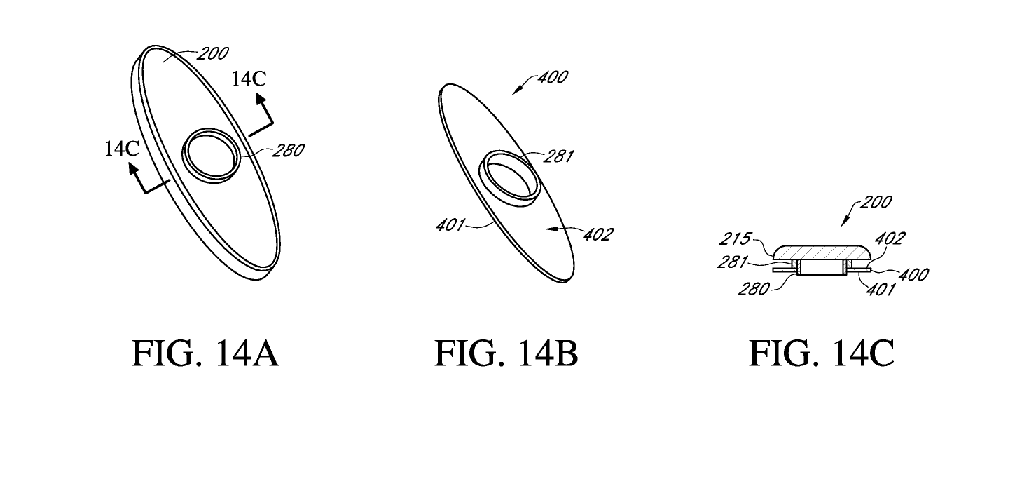

Referring now to FIGS. 14A-14C there is shown an alternative design for affixing the therapy providing device 200 to the patient. In this embodiment, one surface of the therapy providing device includes a first fitting 280 disposed on the second surface 215 of the housing 210. A bandage 400 is provided, wherein the bandage 400 has a proximal surface 402 and a distal surface 401. A biocompatible adhesive/glue layer 425 is disposed on the distal surface of the bandage 401. A second fitting 281 is disposed on the proximal surface 402 of the bandage 400. The first fitting 280 and the second fitting 281 are designed to be received by each other and to form a detachable locking attachment as shown in FIG. 14C. Suitable examples of such detachable fittings may be a screw thread, quarter turn fasteners, grooved pathways, a tapered fitting and the like. A safety lock (not shown) may be incorporated into either of the fittings, wherein the safety lock would engage after the two fittings are brought together in a locking arrangement. The safety lock would prevent the fittings from releasing without an additional application of force or motion to the safety lock to enable the fittings to be separated. The bandage 400 may be a single use product or may be a re-usable bandage as described above. In another aspect, the bandage of the present disclosure, according to some embodiments may be fabricated to include multiple layers, wherein each layer includes a new glue surface. After use, the layer of the bandage having been in contact with tissue is peeled off by the user and properly disposed of, thereby exposing a new glue layer for further use.

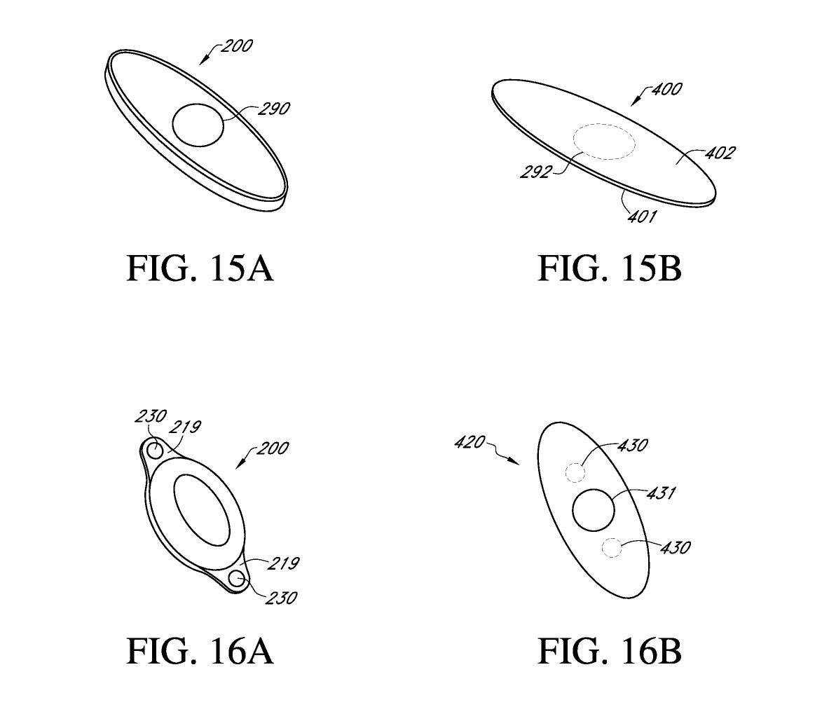

Referring now to FIGS. 15A and 15B there is shown another alternative design for affixing the therapy device 200 to a patient. In this embodiment, a surface of the therapy device 200 includes a magnet 290 disposed thereon or incorporated into the surface. As described above bandage 400 is provided, wherein the bandage 400 has a proximal surface 402 and a distal surface 401. A biocompatible adhesive is disposed on the distal surface of the bandage 401. A metallic member 292 is incorporated into the bandage 400 as shown in FIG. 15B. In use, the user would apply the bandage 400 to their body, wherein the center of the bandage would align with their clavicle. In one embodiment, the bandage 400 would be replaced daily. In another embodiment, the bandage 400 would be reused for a period of time and then replaced. Further still, in another embodiment, the glue surface of the bandage 400 may be refurbished after each use to prolong the useful life of the bandage 400. Once the bandage 400 is affixed to the user, the therapy providing device 200 as shown in FIG. 15A and described above would then be coupled to the bandage through the magnetic coupling between the magnet 290 of the therapy providing device 200 and the metallic member 292 of the bandage 400. It shall be understood that the combination of using a magnet 290 and a metallic member 292 could be reversed. For example, the bandage 400 may contain the magnet 290 and the therapy providing device 200 would have the metallic member 292. Alternatively, both the bandage 400 and the therapy providing device 200 may include a magnet 290, whereby the magnets 290 assist in self-aligning the therapy providing device to the bandage 400. Further still, it is contemplated that the magnet or metallic member of either the bandage 400 or the therapy providing device 200 may be offset from an axis extending through the center of the bandage 400, thereby providing for two different orientations in which the therapy providing device 200 may disposed upon the bandage 400 in for use. Further still, it is contemplated that the driver 220 of the therapy providing device may be offset within the housing 210 from an axis running longitudinally through the housing 210. Offsetting the driver 220 within the housing 210, achieves the same effect of providing multiple mounting orientations of the therapy providing device 200 during use. In yet another embodiment, the magnet 290 or metallic member 292 could be implanted under the user's skin, therefore eliminating the need for the bandage 400. In this embodiment, the therapy providing device 200 could be coupled to the patient's skin directly.

In another embodiment (not shown) the bandage may include an aperture formed therethrough, wherein the metallic member 292 would be disposed about the aperture. The aperture is sized to receive a portion of the therapy providing device 200 therein. It is further contemplated that the therapy providing device may include a second bandage or an enlarged surface similar in size to the bandage 400. The enlarged surface would contain magnets 290 as described above; therefore, when the therapy providing device 200 is disposed within the aperture of the bandage 400, the enlarged surface covers the bandage.

In further embodiments, the magnets and the metallic members may be interchanged, wherein the bandage contains the magnets and the housing may be a metallic member, a portion may be metallic or a portion may be magnetic. Additionally, instead of utilizing magnets and metallic members, other known detachable systems may be utilized, for example a hook and loop configuration or reusable adhesive surface or snaps or the like.

Referring now to FIGS. 16A and 16B there is shown a housing and bandage in accordance with the present disclosure, according to some embodiments, wherein the housing 210 includes extensions or arms 219. The extensions 219 further include magnets 230 disposed therein. Referring now to FIG. 16B there is shown a bandage 420 to be utilized with the housing shown in FIG. 16A. The bandage 420 further includes an aperture 431 formed therethrough, the aperture sized to accept a portion of the therapy providing device 200. The bandage 420 further includes magnets 430 disposed therein. The bandage 420 may be formed of a multilayer construction, wherein the bandage may include a glue layer 425 a glue support layer 423 and a backing layer 421. The aperture may be formed through the entire surface of the bandage or through specific layers of the bandage; for example, the aperture may not be formed through the glue layer. If the aperture is not formed through the glue layer, this provides a biocompatible layer between the user's skin and the vibration source or exciter assembly. It is contemplated that the magnets 430 could be disposed within the glue support layer, wherein the magnets 430 would be encapsulated in the bandage 420 by the glue layer and the backing layer. In use, the user would place the bandage 420 onto their skin, wherein the user can use the aperture 431 to properly align the bandage in the example where the therapy providing device is placed over the clavicle. The user may be able to utilize the aperture to properly align the bandage with their clavicle by placing a finger into the aperture and feeling the clavicle through the glue layer. The glue layer of the bandage 420 may be a re-usable adhesive, such as that described above and commonly utilized on tens electrodes, such as hydrocolloid, hydrogel and silicone adhesives, wherein the glue layer allows for repositioning of the bandage. After placement of the bandage 420, the therapy providing device, having a housing shown in FIG. 16A is disposed over the bandage. The magnets 230 of the housing extensions 219 and the magnets 430 of the bandage act to attach and center the therapy providing device to the bandage. In accordance with the disclosure, it is contemplated that either the magnets 230 of the housing or the magnets 430 of the bandage may be replaced by metallic members or other configurations such a VELCRO, snaps or other detachable structures.

Referring now to FIGS. 16C-16S there are shown additional bandage designs, wherein there are shown the bandages 420′ and 420″ may be utilized with the housings shown and described within the present disclosure, according to some embodiments. Each bandage 420′, 420″, 1420, 3420, 4000, 4041, 5420, and 520 further includes an aperture formed therethrough, the aperture sized to accept a portion of the therapy providing device 200. The bandages may further include magnet members or metallic members or snaps or VELCRO disposed therein or thereon.

As shown in FIG. 16D the bandages 420′ and 420″ may be formed of a multilayer construction, wherein the bandage may include a glue layer 1000, a glue support layer 1002 and a backing layer 1003. It is contemplated that the magnets 430 could be disposed within the glue support layer, wherein the members 430′ and 430″ would be encapsulated between the glue layer and the backing layer, thereby reducing the risk of skin contact. Such protection from skin contact may be beneficial in cases where the members 430, 430′ and 430″ are formed of a metallic material having nickel. Nickel may cause an allergic reaction when placed directly in contact with the skin of certain people. The glue support layer may be formed of a foam material such as a closed cell foam or an open cell foam. The material thickness can be chosen such that the members 430, 430′ and 430″ are sufficiently encapsulated therein. Additionally, the thickness of the glue support later 1002 may be increased or decreased in order to adjust the flexibility of the bandage. Examples of suitable foam material are available from 3M, having product number 1772. The glue support layer may be embodied as a silicone adhesive, also available from 3M (2575P, 2575). Additionally, the density of the foam material may be selected to adjust the flexibility as well. It is contemplated that other materials may be utilized such as rubber, silicon, silicone, paper, fabric, ceramics, polymeric, inorganic materials, honeycomb, fiber reinforced materials.

In accordance with the present disclosure, according to some embodiments, it may be desirable to choose a cover layer, which has elastic properties. The elastic properties of the cover layer may be beneficial in allowing the metallic insert/magnet a degree of movement within the aperture, such that when the bandage is disposed on a user and the therapy providing device is attached to the bandage, the elastic cover layer of the bandage provides some movement of the therapy providing device and the bandage. Additionally, the elasticity of the cover layer allows for the amount of force applied to the user by the therapy providing device to be controlled or adjusted based on material selection. Further still the cover layer may be formed such that it includes a portion of a hook and loop fastener, wherein the therapy providing device would include the second component of the hook and loop fastener, such that when brought together the two would become detachably attached.

In yet another embodiment not shown, the magnet member or metallic members 430 may be directly placed onto the glue later 1000 and then the cover layer 1003 can be directly placed over the top of the glue layer, thereby encapsulating the member 430. In this embodiment the bandage would be composed of only two layers, where the glue support layer 1002 would be eliminated.

In use, the user would place the bandage 420, 420′ and 420″ onto their skin, wherein the user can use the aperture 430, 431′, 431″ to properly align the bandage in the example where the therapy providing device is placed over the clavicle. The user can place the bandage, substantially parallel, parallel, substantially perpendicular or perpendicular to the clavicle. The glue layer of the bandage 420, 420′ or 420″ may be a re-usable adhesive, such as that described above and commonly utilized on tens electrodes, wherein the glue layer allows for repositioning of the bandage. After placement of the bandage 420, 420′ or 420″, the therapy providing device, having a housing shown in FIG. 16A is disposed over the bandage. The magnets 230 of the housing extensions 219 and the magnets or metallic members of the bandage act to attach and center the therapy providing device to the bandage. Additionally, as shown in FIGS. 16C and 16E, the bandages 420′ and 420″ may include a reduced waist portion 432′ and 432″. The reduced waist portion of the bandage may provide the user with another alignment feature in order to properly place the bandage. Further still, the housing of the therapy providing device 200 may also include a reduced waist portion formed therein. In use, the user could “pinch” the therapy providing device using the reduced waist portion to either place or remove the device from the bandage as well as from the charger as previously described above.

Referring now to FIG. 16F, there is shown an embodiment of a bandage 1420 in accordance with the present disclosure, according to some embodiments, wherein the bandage 1420 includes two inserts 1430, wherein the inserts may be metallic members, magnets or magnetic material and an aperture 1431. The bandage 1420 further includes a connecting member 1436, wherein the connecting member is configured to couple the two inserts 1430 together. The connecting member 1436 may be constructed of a metallic member thereby placing the two inserts into electrical communication. The connecting member 1436 may be disposed in a channel formed in the glue support layer or the connecting member 1436 may be disposed onto either side of the glue support layer so long as the connecting member 1436 is capable of connecting the two inserts. In yet another embodiment, the connecting member 1436 may be formed from another type of conductive material such as conductive ink, conductive paint or similar conductive materials that can readily be applied to the glue support layer. The bandage 1420 is constructed in a similar manner to that described above and according to the manufacturing process described below.

Referring now to FIG. 16G, there is shown an embodiment of a bandage in accordance with the present disclosure, the bandage 2420 having similar construction to those described previously. As shown in FIG. 16G, the bandage 2420 includes an aperture 2431 and at least two inserts 2430. In addition, the bandage 2420 includes two pairs of conductive pads 2450 and 2451 disposed on the cover layer of the bandage. The pairs of conductive pads 2450 and 2451, disposed on either side of the aperture 2431, are coupled together either through connective members 2436 or connective member 2438. The connective member 2436 as shown in FIG. 16G is similar to that described above with regard to FIG. 16F, wherein the connective member may be disposed within or upon the glue support layer and may be embodied as a metallic member such as a wire, foil tape etc., or can be constructed of a conductive material such as conductive ink. Alternatively, the conductive pads maybe coupled together with connective members 2438, wherein connective members 2438 can be disposed on the surface of the cover layer of the bandage 2420. The connective member 2438 may be constructed of a metallic member such as a wire or foil tape or may be embodied as a conductive material such as conductive ink. The conductive pads may be constructed of a metallic material such as copper, aluminum, silver, gold or the like or may be constructed of a conductive ink material. Additionally, it is contemplated that at least one pair of conductive pads may additionally be coupled in conductive communication with the inserts 2430 disposed within the glue support layer. Alternatively, one insert 2430 may be conductively coupled to one pair or one conductive pad and the other insert 2430 may be coupled to the other pair or one of the other conductive pads, wherein the inserts 2430 and conductive pads may be utilized to complete an electrical circuit. The conductive pad pairs 2450 and 2451 are configured to engage pins associated with the housing, which then can be utilized to operate the therapy providing device 200.

Referring now to FIG. 16H there is shown another embodiment of a bandage in accordance with the present disclosure. The bandage 3420 shown in FIG. 16H may be constructed according to that previously described and may include features described with reference to other figures. As shown in FIG. 16H the bandage 3420 further includes additional geometric features 3437, shown here to be embodied as generally circular apertures. The additional openings or apertures may be embodied in various geometrical patterns, such as holes, rectangles, squares, triangles or other geometric shapes. Additionally, instead of being formed as cut-outs, the additional openings or apertures maybe formed as slits or spiral cuts formed in the glue support layer. The additional geometric features 3437 as shown in FIG. 16H may be applied to any of the bandages shown and described in accordance with the present disclosure, according to some embodiments. The geometric features 3437 may be formed through all or some of the layers of the bandage 3420 or may be partially formed through at least one layer or multiple layers.

Referring now to FIG. 16I, there is shown an alternative bandage design in accordance with the present disclosure, according to some embodiments. As shown in FIG. 16I, the bandage includes the elements as previously described with regard to the other embodiments. As shown in FIG. 16I, the bandage 4020 includes a generally rectangular portion 4000 and two tab extensions 4005 extending from the generally rectangular portion 4000 and an aperture 4031. The bandage 4020 further includes two inserts 4010. The inserts 4010 maybe magnets, metallic members or other types of magnetic materials.

Referring now to FIG. 16J, there is shown another bandage design. As shown in FIG. 16J, the bandage 4040 is constructed to have a generally star shape profile, wherein the legs 4041 of the bandage 4040 have a generally rounded profile. As shown in FIG. 16J, the bandage includes inserts 4042 similar to those described previously, wherein in this embodiment, the bandage 4040 may include four inserts 4042, by forming the bandage 4040 with the four inserts 4042, this allows for the therapy providing device 200 to be disposed in either orientation on the bandage.

Referring now to FIGS. 16K and 16L there is shown another bandage design in accordance with the present disclosure, according to some embodiments. As shown in the figures, the bandage 5420 is formed of a multilayer construction, wherein the bandage may include a glue layer 5000, a glue support layer 5002 and a backing layer 5003. It is contemplated that the magnets/metallic members 5430 could be disposed within the glue support layer, wherein the magnets/metallic members 5430 would be encapsulated between the glue layer and the backing layer, thereby reducing the risk of skin contact. Unlike other bandage designs described herein, the bandage as shown in FIGS. 16K and 16L is not comprised of continuous layers of materials. As shown, the glue support layer and the cover layer do not extend along the entire length of the bandage, instead these layers terminate adjacent to the “waist” portion 5432 of the bandage 5420. By not extending the glue support layer 5002 and the cover later 5003 along the entire length of the bandage provides for a more flexible bandage.

Referring now to FIG. 16M, there is shown another bandage design in accordance with the present disclosure, according to some embodiments. As shown in FIG. 16M, the bandage further includes a directional element 433 formed into at least one side of the bandage. The directional element 433 may be printed onto the surface of the bandage or formed as apertures or partial cut-outs within a layer or layers of the bandage. The direction element(s) 433 are designed to enable a user to properly align the orientation of the bandage in use. As shown in FIG. 16M, the directional elements 433 are shown to be in the shape of arrows, this should not be considered limiting in any manner. It is contemplated that the directional element may be any shape, such that the shape chosen conveys the orientation of the bandage. Additionally, the directional element may further include a text message such as “this way up, up,” or similar messages that convey direction. It shall be understood that the direction element shown in FIG. 16M and described herein may be utilized with any of the bandage designs shown or described with regard to the present disclosure, according to some embodiments.

Referring now to FIG. 16N there is shown another embodiment of a bandage 420 in accordance with the present disclosure. As shown in FIG. 16N, the aperture 431 is not centered about a centerline of the bandage 420. In this embodiment the aperture 431 may be offset from the bandage centerline by any amount, such that the distance “B” is retained so that the therapy providing device 200 may still be attached to the insert/magnet 430. As shown in FIG. 16N, the inserts/magnets 430 may also be offset about the bandage centerline, so long at the distance “A” between the inserts/magnets 430 is retained to enable the therapy providing device 200 to be attached to the bandage as described herein.

Referring now to FIG. 16O, there is shown another bandage design in accordance with the present disclosure, according to some embodiments. As shown in FIG. 16O, the bandage 420 includes elongated inserts 435 disposed adjacent to the aperture 431. The elongated inserts 435 may be metallic inserts, magnets, magnetic materials, velcro or similar detachable connectors. In this embodiment, the use of the elongated inserts 435 allows for a broader range of placement of the therapy providing device 200 to be coupled to the bandage 420. Therefore, in use, the user can adjust the positioning of the therapy providing device by sliding the therapy providing device along the length of the elongated inserts 435.

Referring now to FIG. 16P there is shown another bandage design in accordance with the present disclosure, according to some embodiments. As shown in FIG. 16P, the bandage includes a directional shape 434 disposed at one end of the bandage. The directional shape 434 aids the user in positioning the bandage as described above with regard to FIG. 16M. It is contemplated that other directional shapes may be utilized other than the specific shape as shown in FIG. 16P. Additionally, the directional shape of FIG. 16 may be combined with any other directional elements disclosed herein, such as those described with regard to FIG. 16M. Additionally, the directional shape 434 may be applied to any other bandage design shown and described herein with regard to the present disclosure, according to some embodiments.

Referring now to FIG. 16Q there is shown yet another bandage design in accordance with the present disclosure, according to some embodiments. As shown in FIG. 16Q, the bandage 520 is constructed in a similar manner to the previously described bandages in accordance with this disclosure. As shown in FIG. 16Q, bandage 520 further includes recesses 532 formed on either side of the aperture 531. The recesses 532 are sized to receive a user's fingertips on the outer edge of the bandage 520. In use, a user grips the bandage 520 between their fingers, wherein their fingers are disposed within the recesses 532. The user can then position the bandage 520 onto their skin, aligning the aperture 532 with their clavicle using their fingertips, which are also gripping the bandage using the recesses 532.

Referring now to FIG. 16R there is shown yet another bandage design in accordance with the present disclosure, according to some embodiments. As shown in FIG. 16R, the bandage 400 further includes directional information 450 disposed on one side of the bandage. The directional information may be printed on the backing layer or proximal surface of the bandage. Alternatively, the directional information may be printed onto the glue support layer, wherein the printed directional information would be visible through the backing layer. As shown in FIG. 16R, the directional information is provided to convey the direction in which the bandage is to be placed onto a user's skin. The direction information can be composed of graphic symbols, such as arrows or may be text information such as “UP, This Way Up, etc.” Additionally, the directional information may include a combination of text and graphics as shown. Further still, as shown in FIG. 16R, the bandage may further include protrusions 410 formed in the waist area 411 of the bandage 400. The protrusions are configured to be received by a user's fingers to aid in handling and/or alignment of the bandage 400. It shall be understood that the protrusions 410 may be added to any of the bandage designs shown within the present disclosure, according to some embodiments and their inclusion only in FIG. 16R should not be considered limiting in any manner.

Referring now to FIG. 16S there is shown another alternative bandage design in accordance with the disclosure. As shown in FIG. 16S, the bandage 400 includes direction information in the form of different colors. That is, one half or one side of the cover layer or proximal surface of the bandage 400 is colored with a first color 455, the second side or half of the bandage 400 is then colored with a second color 456 or does not have any color other than the color of the cover layer material. In use, the user would be instructed as to the direction in which to apply the bandage, for example, if the color 455 is blue, the instructions for use may indicate that the user is to place the blue portion of the bandage closer to their skull.

Referring now to FIGS. 16T and 16U there is shown an alternative bandage design in accordance with some embodiments of the present invention. As shown in FIG. 16T, the bandage 460 is shown as being generally circular in shape, however this should not be considered limiting in any manner and in accordance with the present specification other shapes are contemplated, such as square, rectangular, oval and the like. Referring now to FIG. 16U there is shown a cross-sectional view of the bandage 460 of FIG. 16T. As shown in FIG. 16T the bandage 460 comprises a glue layer 462 and a second layer 461, a metallic insert 292 is disposed between the glue layer 462 and the second layer 461. The metallic insert 292 can be comprised of any magnetic material or comprise a magnet such as a neodymium magnet or similar magnets. The glue layer 462 is configured to be releasably attached to a skin layer of a user and may include a release layer 464 that is disposed over the glue to protect the glue until use. The glue or adhesive on the glue layer may be an acrylic adhesive, a silicon adhesive, a hydrogel, a hydrocolloid or other similar adhesives that can be used to adhere to skin. It is further contemplated that the bandage 460 may include a middle layer (not shown) such as a foam layer, wherein the foam layer may include an aperture to receive the metallic insert 292. The second layer 461 may be a material such as a woven or non-woven layer such as 9907 or 9914 available from 3M.

Referring now to FIG. 16V there is shown an alternative bandage design in accordance with the present specification. As described above with regard to FIGS. 16T and 16U, the bandage of FIG. 16V is constructed of materials similar to those described according to FIG. 16T, however instead of a metallic insert, the bandage 460 shown in FIG. 16V utilizes a snap fitting 465 or similar detachable fitting element attached to at least the second layer 461 of the bandage 460. The therapy providing device 200 would also include a complementary snap-fitting component to interface with the snap fitting 465 disposed on the bandage 460. It is further contemplated that the bandage could be replaced with a currently available EKG electrode bandages, such as those offered by 3M, wherein the therapy providing device 200 would include snap fittings to receive such electrode bandages.

Unlike other bandages described in accordance with some embodiments of the present invention, the bandages described and shown in FIGS. 16T-16X are configured to be utilized in pairs of two for each therapy providing device.

Referring now to FIGS. 16W and 16X, there is shown an alternative bandage design 460 in accordance with some embodiments of the present invention. As shown in FIGS. 16W and 16X, the bandage 460 includes a top layer 463 and a bottom or glue layer 462. In the present embodiment, the top layer 463 includes an adhesive covered by a peel-layer 464. The bottom glue layer 462 is also protected by a peel-layer 464. In use, the user would first remove the peel-layer 464 off of the top layer 463 thereby exposing an adhesive, the exposed adhesive of the bandage would then be attached to one of the flexible arms of the therapy providing device 200. A second bandage 460 would be applied to the second flexible arm of the therapy providing device 200. The user would then remove the peel-layer 464 from the glue layer 462 and then position the system (therapy providing device and two bandages) over their collar bone as described herein.

It is further contemplated that the bandage shown and described in FIGS. 16X and 16W may be utilized in combination with the single piece bandage designs disclosed herein. In this embodiment, the single bandage “dot” shown in FIG. 16X can be re-applied to the circular shaped portion of the unitary bandage as shown in FIGS. 16D and 16E. To apply the dot to the bandage shown in FIG. 16D, the peel-layer 464 would be removed from the bandage dot 460 and then the bandage dot 460 would be applied to the adhesive layer of the bandage of FIG. 16D. The use would repeat the process, using four bandage dots 460 in total on two bandages 420′. After applying the dots 460 to the bandage 420′, the user would then remove the second peel layer 464 from the dots 460 and apply the bandage 420′ to the skin as described herein. The process of applying new bandage dots 460 can be repeated a number of times as desired by the user.

Referring now to FIGS. 16AA, 16BB and 16CC there are shown bottom views of alternative embodiments of a bandage 420 in accordance with the present disclosure. As shown in each of the Figures the bandage 420 may be constructed as described herein, having a top layer, a middle layer and an adhesive, e.g., a glue layer, wherein an insert 430 is disposed between the top layer and the glue layer. In accordance with some embodiments, the glue layer 466 is shown as being a discontinuous layer. As shown in FIG. 16AA, the glue layer 466 is embodied as a plurality of adhesive strips disposed generally horizontally across the bandage shape. As shown in FIG. 16BB, the glue layer 466 is shown as a plurality of adhesive strips disposed generally vertically or offset at an angle across the shape of the bandage. Referring now to FIG. 16CC, in this embodiment, the glue layer 466 is embodied as a plurality of generally circular glue “dots”. In each of these embodiments, the glue layer is embodied as a discontinuous layer, and can be embodied as other geometric shapes or patterns not shown. For example, the glue layer may be triangular, circular, elliptical, hexagonal, square, rectangular, octagonal, hexagonal etc. . . . wherein in each of the embodiments the glue layer is embodied as a discontinuous layer. It is believed that by providing a bandage having a discontinuous glue layer can be beneficial to a user, for example, if the therapy providing device in accordance with the present disclosure, according to some embodiments is intended to be worn daily, utilizing a bandage with a continuous glue layer daily may lead to skin irritation. However, if multiple bandages were offered to a user, each bandage having a different glue layer, it be believed that less skin irritation may occur. For example, on day 1 the user would utilize a bandage having a continuous glue layer, on day 2 they may use a bandage with a discontinuous glue layer, then on day 3 back to a bandage with a continuous glue layer. By alternating the type of bandage, the user may experience less skin irritation. The above example should be considered exemplary in nature, and it shall be understood that the cycle of bandage type used may be varied. In certain embodiments, it may be desirable to select the adhesive bandages such that the pattern on one bandage is offset from the pattern of the bandage intended for use the next day, this way, in use, the adhesive area adhered to the user's skin with the first bandage will not be adhered to with the second bandage, thereby allowing the user's skin to recover to rest from the previous attachment. In some embodiments, the surface area of all of the discontinuous adhesive component is less than about 95%, 90%, 85%, 80%, 75%, 70%, 65%, 60%, 55%, 50%, 45%, 40%, 30% or less relative to the surface area of the top or middle layers.

Referring now to FIGS. 16DD and 16EE, there are shown bottom views of alternative embodiments of a bandage 440 in accordance with the present disclosure, according to some embodiments. As shown in the Figures, the bandage may have a “butterfly design” with a plurality of attachment nodes, such as four nodes that may wrap around a portion of the body to provide optimized attachment to the skin and provide a strong downward pressure of the therapy device 200 on the skin. In some embodiments, the bandage 440 could have a first longitudinal axis, and a second axis orthogonal to the first longitudinal axis. In some embodiments, the nodes could be offset from the first and second axes, such as extending radially outwardly at about a 45 degree angle from the center of the device and with respect to the first and second axes. The bandage may be constructed as described herein, having a top layer, a middle layer and an adhesive, e.g., a glue layer, wherein inserts 445 are disposed between the top layer and the glue layer. The insert 445 may have a radius of distance “G” as shown in FIG. 16EE. Some embodiments may also have an aperture 446 for receiving the therapy device, which can be centrally located along both the axial and longitudinal midlines of the bandage 440, or in other locations. The aperture 446 may have any desired geometry, but in some embodiments can have a non-rectangular configuration such that it includes two parallel or substantially parallel borders spaced apart at a distance “C” and two curved borders that are a portion of a circle and form a radius “H.” In some embodiments, the inserts may be spaced apart such that distances “A” and “B” may be either equivalent or different, in order to alter the shape as either a “square” configuration or “rectangular” configuration. The nodes may further be configured so that the inserts 445 are equivalently spaced and radial distances “D,” “E,” and “F” are the same. Alternatively, the inserts 445 may be offset from the node centerlines such that the radial distances “D,” “E,” and “F” are different. For example, one radial distance could be about, less than about, or no more than about 5%, 10%, 15%, 20%, 30%, 40%, 50%, 75%, or 100% more or less than another radial distance. Each of these distances can be calculated such that the inserts/magnets 445/230 are retained to enable the therapy providing device 200 to be attached to the bandage 440 as described herein.

Referring now to FIGS. 16FF (showing a therapy device attached to a patient's clavicle) and 16GG (showing the bandage attached to a patient's clavicle, without the therapy device), there are shown images of an embodiment of a therapy device 200 affixed to a patient with a suitable attachment such as magnets 230 operably connected to a bandage, such as as depicted in FIG. 16DD above. As shown, two of the nodes rest on one side of the collarbone, and the other two nodes rest on the other side of the collarbone, such that the bandage effectively wraps around the collarbone and allows the therapy device to advantageously exert additional downward force and energy on the clavicle.

It is further contemplated in accordance with the present disclosure that the bandage designs of some embodiments of the present invention could also incorporate one, two, or more biologically active compounds, which when applied to the user would be delivered topically, such as transdermally. The term “biologically active compound” refers to any chemical substance that has a biological effect in humans or animals for medical, therapeutic, cosmetic and veterinary purposes, and encompasses pharmaceuticals including drugs, cosmeceuticals, nutraceuticals, and nutritional agents. It will be appreciated that some of biologically active compounds can be classified in more than one of these classes.

The biologically active compound may be incorporated into the glue layer or may be interposed within the glue layer. For example, areas of glue from the glue layer may be removed, wherein the active compound and any necessary substrate can be disposed in place of the removed glue, thereby forming a hybrid glue/beneficial agent layer. Further still, a separate active compound layer may be included in the design of the bandage, wherein the separate layer may be interposed in an order, for example behind the glue layer such that the active compound would not be in direct contact with the user's tissue. It will be appreciated that in some embodiments, while the bandage design in general may incorporate a biologically active compound, the portion of the bandage design configured to receive the therapy device 200 may not contain the biologically active compound. For instance, in some bandage design embodiments including an aperture where the therapy device 200 is placed will not include the biologically active compound at the aperture. For example, a bandage could have a skin-contacting surface defining a surface area, and the surface area may not, in some embodiments, be completely covered by the active compound. The active compound could, in some embodiments, be contained over about or less than about 90%, 85%, 80%, 75%, 70%, 65%, 60%, 55%, 50%, 45%, 40%, 30%, 20%, 10%, or less of the total surface area of the skin-contacting surface. In other words, the skin-contacting surface of the bandage could include one, two, or more windows that are free or substantially free of the biologically active compound. This can be advantageous, in some embodiments, to allow the therapy device to create a synergistic effect via a mechanism of action (e.g., an effect of baroreceptors, for example) that could potentially be different/unrelated to increasing penetration/absorption of the biologically active compound. For example, the therapy device could emit energy, such as vibration/ultrasound energy through the skin surface through a direct path where the active compound may not necessarily be present, and via a different mechanism unrelated to the active compound, such as modulation of the baroreceptors. Instead, in such embodiments the aperture could have only a very thin protective layer of adhesive patch such that the therapy device 200 may be in close contact with the skin of a user.

A wide range of biologically active compounds may be delivered transdermally with the bandage of some embodiments of the present invention. Examples include, but are not limited to, cardiovascular drugs, in particular antihypertensive agents (e.g. calcium channel blockers or calcium antagonists) and antiarrhythmic agents; congestive heart-failure pharmaceuticals; inotropic agents; vasodilators; ACE inhibitors; diuretics; carbonic anhydrase inhibitors; cardiac glycosides; phosphodiesterase inhibitors; alpha-blockers; β-blockers; sodium channel blockers; potassium channel blockers; β-adrenergic agonists; platelet inhibitors; angiotensin II receptor antagonists; anticoagulants; thrombolytic agents; treatments for bleeding; treatments for anemia; thrombin inhibitors; antiparasitic agents; antibacterial agents; insulin; human growth hormone and peptides; vaccines; anti-inflammatory agents, including non-steroidal anti-inflammatory agents (NSAIDs), such as COX-2 inhibitors; steroidal anti-inflammatory agents; prophylactic anti-inflammatory agents; antiglaucoma agents; mast cell stabilizers; mydriatics; agents affecting the respiratory system; allergic rhinitis pharmaceuticals; alpha-adrenergic agonists; corticosteroids; chronic obstructive pulmonary disease pharmaceuticals; xanthine-oxidase inhibitors; antiarthritis agents; gout treatments; autacoids and autacoid antagonists; antimycobacterial agents; antifungal agents; antiprotozoal agents; anthelmintic agents; antiviral agents especially for respiratory, herpes, cytomegalovirus, human immunodeficiency virus and hepatitis infections; treatments for leukemia and Kaposi's sarcoma; pain management agents in particular opioids, anaesthetics and analgesics; neuroleptics; sympathomimetic pharmaceuticals; adrenergic agonists; drugs affecting neurotransmitter uptake or release; anticholinergic pharmaceuticals; antihemorrhoid treatments; agents to prevent or treat radiation or chemotherapeutic effects; lipogenesis drugs; fat reducing treatments; anti-obesity peptides; antiobesity agents such as lipase inhibitors; sympathomimetic agents; treatments for gastric ulcers and inflammation such as proton pump inhibitors; prostaglandins; VEGF inhibitors; antihyperlipidemic agents, in particular statins; drugs that affect the central nervous system (CNS) such as antipsychotic, antiepileptic and antiseizure drugs (anticonvulsants), psychoactive drugs, stimulants, antianxiety and hypnotic drugs, antidepressant drugs; anti-Parkinson's pharmaceuticals; hormones and fragments thereof such as sex hormones; growth hormone antagonists; gonadotropin releasing hormones and analogues thereof; steroid hormones and their antagonists; selective estrogen modulators; growth factors; antidiabetic pharmaceuticals such as insulin, insulin fragments, insulin analogues, glucagon-like peptides and hypoglycemic agents; H1, H2, H3 and H4 antihistamines; peptide, protein, polypeptide, nucleic acids and oligonucleotide pharmaceuticals; analogues, fragments and variants of natural proteins, polypeptides, oligonucleotides and nucleic acids and such like compounds; agents used to treat migraine headaches; asthma pharmaceuticals; cholinergic antagonists; glucocorticoids; androgens; antiandrogens; inhibitors of adrenocorticoid biosynthesis; osteoporosis treatments such as bisphosphonates; antithyroid pharmaceuticals; sunscreens, sun protectants and filters; cytokine agonists; cytokine antagonists; anticancer drugs; anti-Alzheimer's drugs; HMG-CoA reductase inhibitors; fibrates; cholesterol absorption inhibitors; HDL cholesterol elevating agents; triglyceride reducing agents; anti-aging or antiwrinkle agents; precursor molecules for the generation of hormones; proteins such as collagen and elastin; antibacterial agents; anti acne agents; antioxidants; hair treatments and skin whitening agents; sunscreens, sun protectants and filters; variants of human apolipoprotein; precursor molecules for generation of hormones; proteins and peptides thereof; amino acids; plant extracts such as grape seed extract; DHEA; isoflavones; nutritional agents including vitamins, phytosterols and iridoid glycosides, sesquiterpene lactones, terpenes, phenolic glycosides, triterpenes, hydroquinone derivatives, phenylalkanones; antioxidants such as retinol and other retinoids including retinoic acid and co enzyme Q10; omega-3-fatty acids; glucosamine; nucleic acids, oligonucleotides, antisense pharmaceuticals; enzymes; cytokines; cytokine analogues; cytokine agonists; cytokine antagonists; immunoglobulins; antibodies; antibody pharmaceuticals; gene therapies; lipoproteins; erythropoietin; vaccines; small and large molecule therapeutic agents for the treatment, or prevention of human and animal diseases such as allergy/asthma, arthritis, cancer, diabetes, growth impairment, cardiovascular diseases, inflammation, immunological disorders, baldness, pain, ophthalmological diseases, epilepsy, gynaecological disorders, CNS diseases, viral infections, bacterial infections, parasitic infections, GI diseases, obesity, and hematologic diseases.

Some specific non-limiting examples of suitable biologically active compounds include:

Anaesthetics: including amino-ester and amino-amide anaesthetics such as benzocaine, chloroprocaine, cocaine, reserpine, guanethidine, cyclomethycaine, dimethocaine/larocaine, propoxycaine, procaine/novocaine, proparacaine, tetracaine/amethocaine; articaine, bupivacaine, carticaine, cinchocaine/dibucaine, etidocaine, levobupivacaine, lidocaine/lignocaine, mepivacaine, piperocaine, prilocaine, ropivacaine, trimecaine, propofol, halothane, enflurane barbiturates, benzodiazepines, neostigmine and ketamine

Alkylating Agents: including carmustine, cyclophosphamide, ifosfamide, streptozotocin and mechlorethamine

Calcium Channel Blockers: including amlodipine, aranidipine, azelnidipine, barnidipine, benidipine, cilnidipine, clevidipine, cronidipine, darodipine, dexniguldipine, efonidipine, elnadipine, elgodipine, felodipine, flordipine, furnidipine, iganidipine, isradipine, lacidipine, lemildipine, lercanidipine, manidipine, mesuldipine, nicardipine, nifedipine, niludipine, nilvadipine, nimodipine, nisoldipine, nitrendipine, olradipine, oxodipine, palonidipine, pranidipine, sagandipine, sornidipine, teludipine, tiamdipine, trombodipine, watanidipine, verapamil, gallopamil, benzothiazepine, diltiazem, mibefradil, bepridil, fluspirilene and fendiline

Antiarrhythmic and Antiangina Agents: including amiodarone, bretylium, disopyramide, flecainide acetate, quinidine sulfate, nitroglycerine, ranolazine, amiodarone, isosorbide, propafenone, sotalol, droneadrone, dofetilide, and alteplase

Antibacterial, Antibiotic and Antiacne Agents: including amoxicillin, ampicillin, azithromycin, benethamine penicillin, bleomycin, benzoyl peroxide, cinoxacin, chloramphenicol, daunorubicin, plicamycin, fluoroquinolones, ciprofloxacin, clarithromycin, clindamycin, clindesse, clofazimine, chlorohexidine gluconate, cloxacillin, demeclocycline, doxycycline, erythromycin, ethionamide, imipenem, indomethacin, lymocycline, minocycline, nalidixic acid, nitrofurantoin, penicillin, rifampicin, spiramycin, sodium sulfacetamide, sulphabenzamide, sulphadoxine, sulphamerazine, sulphacetamide, sulphadiazine, sulphafurazole, sulphamethoxazole, sulphapyridine, tetracycline, cephalexin, cefdinir, triclosan, ofloxacin, vancocin, glyburide, mupirocin, cefprozil, cefuroxime axetil, norfloxacin, isoniazid, lupulone, D-penicillamine, levofloxacin, gatifloxacin, and trimethoprim

Anticancer: including doxorubicin, 6-thioguanine, paclitaxel, docetaxel, camptothecin, megestrol acetate, navelbine, cytarabine, fludarabine, 6-mercaptopurine, 5-fluorouracil, teniposide, vinblastine, vincristine, cisplatin, colchicine, carboplatin, procarbazine and etoposide.

Antidepressants, Antipsychotics and Antianxiety: including alprazolam, amoxapine, bentazepam, bromazepam, clorazipine, clobazam, clotiazepam, diazepam, lorazepam, flunitrazepam, flurazepam, lormetazepam, medazepam, nitrazepam, oxazepam, temazepam, maprotiline, mianserin, nortriptyline, risperidone, sertraline, trazodone, baloperidol, trimipramine maleate fluoxetine, ondansetron, midazolam, chlorpromazine, haloperidol, triazolam, clozapine, fluopromazine, fluphenazine decanoate, fluanisone, perphenazine, pimozide, prochlorperazine, sulpiride, thioridazine, paroxitine, citalopram, bupropion, phenelzine, olanzapine, divalproex sodium and venlafaxine

Tricyclics: including azothiopine, amitriptyline, famotidine, promethazine, paroxetine, oxcarbazapine and mertazapine

Antidiabetics: including acetohexamide, chlorpropamide, glibenclaraide, gliclazide, glipizide, metformin, tolazamide, glyburide, glimepiride, tolbutamide, glitazones, or DPP-4 inhibitors.

Antiepileptics: including beclamide, carbamazepine, gapapentin, tiagabine, vigabatrin, topiramate, clonazepam, ethotoin, methoin, methsuximide, methylphenobarbitone, oxcarbazepine, paramethadione, phenacemide, phenobarbitone, phenytoin, phensuximide, primidone, sulthiamine, phenytoin sodium, nirofurantoin monohydrate, gabapentin, lamotrigine, zonisamide, ethosuximide and valproic acid

Hypnotics/Sedatives and Muscle Relaxants: including zolpidem tartrate, amylobarbitone, barbitone, butobarbitone, pentobarbitone, brotizolam, carbromal, chlordiazepoxide, chlormethiazole, ethinamate, meprobamate, methaqualome, cyclobenzaprene, cyclobenzaprine, tizanidine, baclofen, butalbital, zopiclone, atracurium, tubocurarine and phenobarbital.

Antifungal, Antiprotazoal and Antiparasitic Agents: including amphotericin, butoconazole nitrate, clotrimazole, econazole nitrate, fluconazole, flucytosine, griseofulvin, itraconazole, ketoconazole, miconazole, natamycin, nystatin, sulconazole nitrate, terconazole, tioconazole and undecenoic acid; benznidazole, clioquinol, decoquinate, diiodohydroxyquinoline, diloxanide furoate, dinitolmide, furzolidone, metronidazole, nimorazole, nitrofurazone, ornidazole, terbinafine, clotrimazole, chloroquine, mefloquine, itraconazole, pyrimethamine, praziquantel, quinacrine, mebendazole and timidazole

Antihypertensive and/or Cardiac Therapeutic Agents: including candesartan, hydralazine, clonidine, triamterene, felodipine, gemfibrozil, fenofibrate, nifedical, prazosin, mecamylamine, doxazosin, dobutamine and cilexetil

Antimigraine Agents: including dihydroergotamine mesylate, ergotamine tartrate, methysergide maleate, pizotifen maleate and sumatriptan succinate

Antimuscarinic Agents: including atropine, benzhexyl, biperiden, ethopropazine, hyoscyamine, mepenzolate bromide, oxybutynin, oxyphencylcimine and tropicamide

Antineoplastic Agents (or Immunosuppressants): including aminoglutethimide, amsacrine, azathioprine, busulphan, chlorambucil, cyclosporin, dacarbazine, estramustine, etoposide, lomustine, melphalan, mercaptopurine, methotrexate, mitomycin, mitotane, mitozantrone, procarbazine, tamoxifen citrate, testolactone, tacrolimus, mercaptopurine and sirolimus

Antiparkinsonian Agents: including bromocriptine mesylate, levodopa, tolcapone, ropinirole, bromocriptine, hypoglycaemic agents such as sulfonylureas, biguanides, α-glucosidase inhibitors, thaiazolidinediones, cabergoline, carbidopa and lysuride maleate

Antithyroid Agents: including carbimazole, methimazole, and propylthiouracil. Some embodiments can also include prothyroid/thyroid replacement agents, such as levothyroxine or liothyronine.

Antiviral Drugs: including amantadine, retinovir, cidofovir, acyclovir, famciclovir, ribavirin, amprenavir, indinavir, rimantadine and efavirenz, penciclovir, ganciclovir, vidarabine, abacavir, adefovir, amprenavir, delavirdine, didanosine, stavudine, zalcitabine, zidovudine, enfuvirtide and interferon

Cardiac Inotropic Agents: including aminone, milrinone, digitoxin, digoxin, enoximone, lanatoside C and medigoxin.

### Lipid-Modifying and Anti-Obesity Agents:

including fenofibrate, clofibrate, probucol, ezetimibe and torcetrapib. Also anti-obesity agents may be used, e.g., orlistat.

Anti-inflammatory: including meloxicam, triamcinolone, cromolyn, nedocromil, hydroxychloroquine, montelukast, zileuton, zafirlukast and meloxicam

Antihistamine: including diphenhydramine, fexofenadine, chloral hydrate, hydroxyzine, promethazine, cetirazine, cimetidine, cyclizine, meclizine, dimenhydrinate, loratadine, nizatadine and promethazine.

Antiulcer: including omeprazole, lansoprazole, pantoprazole and ranitidine

Diuretics: including hydrochlorothiazide, amiloride, acetazolamide, bumetamide, furosemide, metolazone, ethacrynic acid, and torsemide.

Opioids: including natural opiates which are alkaloids contained in the resin of the opium poppy such as morphine, codeine and thebaine; semi-synthetic opioids created from natural opiates such as hydromorphone, hydrocodone, oxycodone, oxymorphone, desomorphine, diacetylmorphine (heroin), nicomorphine, dipropanoylmorphine, benzylmorphine and ethylmorphine; fully synthetic opioids such as fentanyl, pethidine, methadone, tramadol and dextropropoxyphene; and, endogenous opioid peptides, produced naturally in the body, such as endorphins, enkephalins, dynorphins, and endomorphins; opioid analgesics including opioid receptor agonists, opioid receptor partial agonists, opioid antagonist or opioid receptor mixed agonist-antagonists; opioid receptor agonists including morphine, depomorphine, etorphine, heroin, hydromorphone, oxymorphone, levorphanol, methadone, levomethadyl, meperidine, fentanyl, sufentanyl, alfentanil, codeine, hydrocodone, oxycodone, and mixtures of the foregoing; opioid receptor antagonists including naloxone and naltrexone; opioid receptor mixed agonist-antagonist which has mixed opioid agonist/antagonist activities, or one that exhibits only partial agonist activity, including buprenorphine, nalbuphine, butorphanol, pentazocine, and mixtures of such compounds; opioids which exhibit partial agonist activity, including ethylketocyclazocine; opium alkaloids including phenanthrenes which are naturally occurring in opium such as codeine, morphine, thebaine and oripavine (the active metabolite of thebaine); synthetic derivatives such as diacetylmorphine (heroin), dihydrocodeine, hydrocodone, hydromorphone, nicomorphine, desmorphine, ethylmorphine, dipropanoylmorphine, oxycodone and oxymorphone; synthetic opioids including anilidopiperidines such as fentanyl, alphamethylfentanyl, alfentanil, sufentanil, remifentanil, carfentanyl and ohmefentanyl, Phenylpiperidines such as pethidine (meperidine), ketobemidone, MPPP, allylprodine, prodine and PEPAP; diphenylpropylamine derivatives such as propoxyphene, dextropropoxyphene, dextromoramide, bezitramide, piritramide, methadone, dipipanone, levomethadyl acetate (LAAM), difenoxin, diphenoxylate and loperamide; benzomorphan derivatives such as dezocine, pentazocine and phenazocine; oripavine derivatives such as buprenorphine, dihydroetorphine and etorphine; morphinan derivatives such as butorphanol, nalbuphine, levorphanol and levomethorphan, and others such as lefetamine, meptazinol, tilidine, tramadol and tapentadol; opioid receptor antagonists including nalmefene, naloxone and naltrexone

NSAIDs: including arylalkanoic acid sub-group of class which includes diclofenac, aceclofenac, acemetacin, alclofenac, bromfenac, etodolac, indometacin, indometacin farnesil, nabumetone, oxametacin, proglumetacin, sulindac and tolmetin; 2-arylpropionic acid (profens) sub-group of class which includes alminoprofen, benoxaprofen, carprofen, dexibuprofen, dexketoprofen, fenbufen, fenoprofen, flunoxaprofen, flurbiprofen, ibuprofen, ibuproxam, indoprofen, ketoprofen, ketorolac, loxoprofen, miroprofen, naproxen, oxaprozin, pirprofen, suprofen, tarenflurbil and tiaprofenic acid; and N-arylanthranilic acid (fenamic acid) sub-group of class which includes flufenamic acid, meclofenamic acid, mefenamic acid and tolfenamic acid; tromethamine, celecoxib, nepafenac, aspirin, rofecoxib, naproxen, sulindac, piroxicam, pheylbutazone, tolmetin, indomethacin, acetaminophen (paracetamol), tramadol and propoxyphene.

Retinoids: including first generation retinoids such as retinol, retinal, tretinoin (retinoic acid, Retin-A), isotretinoin and alitretinoin; second generation retinoids such as etretinate and its metabolite acitretin; third generation retinoids such as tazarotene, bexarotene and adapalene

Hormones and Steroids: including adrenocorticotrophic hormone (ACTH), antidiuretic hormone (vasopressin), atrial-natriuretic factor (ANF), atrial-natriuretic peptide (ANP), beclomethasone, cortisone, scopolamine, dopamine, epinephrine, catecholamines, cholecystokinin, clomiphene citrate, danazol, dexamethasone, diethylstilbestrol (DES), ethinyl estradiol, fludrocortisone, finasteride, follicle stimulating hormone, gastrin, hydroxyprogesterone, growth hormone, insulin, leptin, luteinizing hormone, medroxyprogesterone acetate, mestranol, quinestrol, methyltestosterone, nandrolone, norethindrone, norethisterone, norgestrel, estradiol, conjugated oestrogens, oxandrolone, oxytocin, prednisone, progesterone, prolactin, prostaglandins, somatostatin, testosterone, stanozolol, stibestrol, thyroxine, prednisolone phosphate, triamcinolone, mifepristone acetonide, budesonide, levothyroxine, testosterone, testosterone cypionate, fluoxymesterone, flutamide, mometasone furoate, cyproterone, fluoromethalone, goserelin, leuprolide, calcitonin, halobetasol, hydrocortisol and tibolone

Statins and Derivatives: including atorvastatin, fluvastatin, lovastatin, nystatin, rosuvastatin, pravastatin, orlistat and simvastatin

Stimulants: including amphetamine, phentermine, tyramine, ephedrine, metaraminol, phenylephrine, dexamphetamine, dexfenfluramine, fenfluramine, nicotine, caffeine and mazindol

Vasocontrictors: including desmopressin and vasopressin.

### Vasodilators:

including carvedilol, terazosin, phentolamine, minoxidil, isosorbide, and menthol.

### Anti-Alzheimer's Agents:

including levetiracetam, levitiracetam and donepezil

ACE Inhibitors: including benazepril, enalapril, ramipril, fosinopril sodium, lisinopril, minoxidil, isosorbide, rampril and quinapril

Beta Adrenoreceptor Antagonists: including atenolol, timolol, pindolol, propanolol hydrochloride, bisoprolol, esmolol, carvedilol metoprolol succinate, metoprolol and metoprolol tartrate

Angiotensin II Antagonists (angiotensin II receptor blockers, or ARBs): including losartan, candesartan, and irbesartan.

Platelet Inhibitors: including abciximab, clopidogrel, tirofiban and aspirin.

### Alcohols and Phenols:

including tramadol, tramadol hydrochloride, allopurinol, calcitriol, cilostazol, sotalol, urasodiol bromperidol, droperidol, flupenthixol decanoate, albuterol, albuterol sulphate, carisoprodol, clobetasol, ropinirol, labetalol, and methocarbamol.

Ketones and Esters: including amiodarone, fluticasone, spironolactone, prednisone, triazodone, desoximetasone, methyl prednisolone, benzonatate nabumetone and buspirone.

Antiemetics: including metoclopramide, ondansetron, promethazine, and droperidol.

Anticoagulant and Antithrombotic Agents: including warfarin, enoxaparin, fondaparinux, heparin, hirudin, rivaroxaban, dabigatran, and lepirudin.

Treatments for Gout: including probenecid, colchicine, allopurinol and sulfinpyrazone

COPD and Asthma Treatments: including albuterol, salmeterol, ipratropium, theophylline, aminophylline, leukotriene modifiers, omalizumab, and steroidal agents

Treatments for Osteoporosis: including raloxifene, pamidronate and risedronate

Cosmetic Peptides: including acetyl hexapeptide-3, acetyl hexapeptide-8, acetyl octapeptide and I-carnosine

Vaccines: including vaccines comprising toxoids (inactivated toxic compounds); proteins, protein subunits and polypeptides; polynucleotides such as DNA and RNA; conjugates; adjuvants such as saponins, virosomes, inorganic and organic adjuvants, for example zostavax

Nutraceutical and Cosmeceutical Actives: including coenzyme Q₁₀ (or ubiquinone), ubiquinol or resveratrol; a carotenoid such as α, β, or γ-carotene, lycopene, lutein, zeaxanthin and astaxanthin; a phytonutrient, such as lycopene, lutein and seaxanthin; an unsaturated fatty acid such as linoleic acid, conjugated linoleic acid, linolenic acid, omega-3 fatty acids including but not limited to docosahexaenoic acid (DHA) and eicosapentaeonic acid (EPA) and their glycerol-esters; fat-soluble vitamins including vitamin D (D2, D3 and their derivatives), vitamin E (α, β, γ, δ-tocopherols, or α, β, γ, δ-tocotrienols), vitamin A (retinol, retinal, retinoic acid and derivatives), vitamin K (K₁, K₂, K₃ and their derivatives) capric/caprylic triglycerides, folic acid, iron, niacin, glyceryl linoleate, omega 6 fatty acids, vitamin F, selenium, cyanocobalamin, aloe vera, beta glucan, bisabolol, camellia thea (green tea) extract, capric/caprylic triglycerides, centella asiatica (gotu cola) extract, cetearyl olivate, chlorophyll, citrus sinensis (orange) oil, cocoyl proline, dicapryl ether, disodium lauriminodipropionate tocopheryl phosphates (vitamin E phosphates), glycerin, glyceryl oleate, glycyrrhiza glabra (licorice) root extract, hamamelis virgiana (witch hazel) extract, lactic acid, lecithin, lutein, macadamia integrifolia (macadamia) seed oil, matricaria chamomilla (chamomile) extract, oenothera biennis (evening primrose) oil, olea europaea (olive) leaf extract, rice bran oil, persea gratissima (avocado) oil, polygonum multiflorum extract, pomegranate sterols, resveratrol, rosa eglanteria (rose hip) oil, santalum spicatum (sandalwood) oil, titanium dioxide, folic acid, glycerin, glyceryl linoleate (omega 6 fatty acids vitamin F), vitamin A palmitate, vitis vinifera (grapeseed) oil, halobetasol, adenosine, adenosine triphosphate, alpha hydroxy acid, allantoin, hyaluronic acid and derivatives, isolutrol, tranexamic acid, glycolic acid, arginine, ascorbyl glucosamine, ascorbyl palmitate, salicylic acid, carnosic acid, alpha lipoic acid, gamma linolenic acid (GLA), panthenol, retinyl propionate, retinyl pamitate, furfuryladenine, retinaldehyde, copper peptides, idebenone, dimethylaminoethanol (DMAE), niacinamide, beta-glucan, palmitoyl pentapeptide-4, palmitoyl oligopeptide/tetrapetide-7, ethocyn, ceramides, phenylalanine, glucuronolactone, L-carnitine, hydroxylapetite, palmitoyl tripetide-3, forskolin, zinc oxide, α-bisabolol, eugenol, silybin, soy isoflavones, aucubin, catalpol, pseudoguaianolide from Arnica chamissonis, rosmarinic acid, rosmanol, salicylates for example salicin, saligenin and salicyclic acid, taxasterol, α-lactucerol, isolactucerol, taraxacoside, ceremides, arbutin, gingerols, shagaols, hypercin, elastin, collagen and peptides thereof.

Particularly preferred biologically active compounds include alprazolam, donepezil, risperidone, lorazepam, nicotine, lidocaine, diclofenac, felodipine, insulin, ketoralac, prilocalne, halobetasol, hydrocortisol, opioids such as oxycodone or dihydrohydroxycodeinone (oxycodone base).

Adrenergic stimulants such as fenoterol, isoprenaline, orciprenaline, rimeterol, salbutamol, salmeterol, terbutaline, dobutamine, phenylephrine, phenylpropanolamine, pseudoephedrine may find particular utility in treating sleep disorders or jet lag or sickness. Other stimulants which may find utility with the devices and methods of some embodiments of the present invention may include Cocaine, Dextroamphetamine (Dexedrine), Methamphetamine (Desoxyn), Methylphenidate (Ritalin), Phenmetrazine (Preludin), Biphetamine, Benzphetamine, Didrex, Chlorphentermine, Clortermine, Phendimetrazine tartrate (Plegine, Prelu 2), Norpseudoephedrine, Diethylpropion hydrochloride (Tenuate), Fencamfamin, Fenproporex, Phentermine (Fastin, Ionamin, Adipex), Mazindol (Sanorex, Mazanor), Mefenorex, Modafinil (Provigil), Pemoline (Cylert (No longer available in U.S.)), Pipradrol, Sibutramine (Meridia), Pyrovalerone, Diethylpropion, Fenproporex, Phentermine, Mazindol, Modafinil, Pemoline and Sibutramine.

In some embodiments, the biologically active compound comprises an anti-hypertensive agent. The anti-hypertensive agent could be a transdermal form of clonidine such as that included in the CATAPRES-TTS patch. In some embodiments, the bandage including clonidine could have a delivered dose of between about 0.1 mg and about 0.6 mg over 24 hours, such as about 0.1 mg, 0.2 mg, 0.3 mg, 0.4 mg, 0.5 mg, or 0.6 mg, or various ranges including two of the aforementioned values. The bandage including clonidine could be replaced at a desired interval according to the desired clinical result, such as daily or weekly for example.

In some embodiments, the biologically active compound comprises nitroglycerin. In some embodiments, the bandage including nitroglycerin could have a delivered dose of between about 0.1 mg and about 1.0 mg per hour, such as about 0.1 mg, 0.2 mg, 0.3 mg, 0.4 mg, 0.5 mg, 0.6 mg, 0.7 mg, 0.8 mg, 0.9 mg, or 1.0 mg, or various ranges including two of the aforementioned values. The bandage including nitroglycerin could be replaced at a desired interval according to the desired clinical result, such as a daily application for 12 to 14 hours daily. In some embodiments, the bandage includes nitropaste of which ¾″, ½″, ¾″, 1″, 1.5″, 2″ or various ranges including two of the aforementioned values are applied at a desired interval according to the desired clinical result, such as every 4, 6, or 8, 12, 16, or 24 hours for example.

In some embodiments, the biologically active compound is one or more of the following: timolol maleate, nicardipine hydrochloride, captopril, atenolol, metoprolol tartrate, clonidine, indapamide, labetalol, pinacidil, verapamil, nitrendipine, nifedipine, nicorandil, propranolol, diltiazem, amlodipine besylate, carvedilol, and/or lisinopril dehydrate.

It is to be understood that pharmaceutically, nutraceutically and/or cosmeceutically acceptable derivatives of biologically active compounds are included within the scope of some embodiments of the present invention.

The term “pharmaceutically, nutraceutically or cosmeceutically acceptable derivatives” includes, but is not limited to, pharmaceutically, nutraceutically or cosmeceutically acceptable salts, esters, salts of such esters, ethers, or any other derivative including prodrugs and metabolites, which upon administration to a subject (e.g. patient, human or animal) in need is capable of providing, directly or indirectly, a biologically active compound as otherwise described herein.

As used herein, the term “pharmaceutically, nutraceutically or cosmeceutically acceptable salt” refers to those salts which are, within the scope of sound medical judgment, suitable for use in contact with the tissues of humans and lower animals without undue toxicity, irritation, allergic response and the like, and are commensurate with a reasonable benefit/risk ratio. Pharmaceutically, nutraceutically or cosmeceutically acceptable salts are well known in the art. For example, S. M. Berge, et al. describe pharmaceutically, nutraceutically or cosmeceutically acceptable salts in detail in J. Pharmaceutical Sciences, 66:1-19, 1977. Examples of pharmaceutically, nutraceutically or cosmeceutically acceptable nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid, or malonic acid or by using other methods used in the art such as ion exchange. Other pharmaceutically acceptable salts include adipate, alginate, ascorbate, aspartate, benzenesulfonate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hernisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. Representative alkali or alkaline earth metal salts include sodium, lithium, potassium, calcium, magnesium, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate, and aryl sulfonate.

The term “pharmaceutically, nutraceutically or cosmeceutically acceptable ester” refers to esters which are hydrolysed in vivo and include those that break down readily in the human body to leave the parent compound or a salt thereof. Suitable ester groups include, for example, those derived from pharmaceutically, nutraceutically or cosmeceutically acceptable aliphatic carboxylic acids, particularly alkanoic, alkenoic, cycloalkanoic and alkanedioic acids, in which each alkyl or alkenyl moiety advantageously has not more than 6 carbon atoms. Examples of particular esters include formates, acetates, propionates, butyrates, acrylates and ethylsuccinates.

The term “pharmaceutically, nutraceutically or cosmeceutically acceptable prodrugs” as used herein refers to those prodrugs of the biologically active compounds which are, within the scope of sound medical judgment, suitable for use in contact with the tissues of a subject with undue toxicity, irritation, allergic response, and the like, commensurate with a reasonable benefit/risk ratio, and effective for their intended use, as well as the zwitterionic forms, where possible, of the compounds of the invention. The term “prodrug” refers to compounds that are rapidly transformed in vivo to yield the parent compound of the above formula, for example by hydrolysis in blood.

Some embodiments of the present invention are further not limited solely to the administration of one biologically active compound: more than one biologically active compound or other therapeutic compounds may be incorporated into the bandage.

The biologically active compound may be present in a therapeutically effective amount, that is, an amount necessary to achieve a desired therapeutic effect.

It shall be understood that the various features of the multiple bandage embodiments maybe combined together into a single embodiment and that each bandage figure should not be considered limiting in any manner.

In another embodiment, not shown, it is contemplated that certain components/parts of the therapy providing system as described herein may be located in different locations. For example, it is contemplated that the bandage may be designed to include the energy source, thereby eliminating the need for an energy source to be disposed within the housing of the therapy providing device 210. By removing the battery from the housing, the housing could be reduced in size. In this embodiment, the bandage may be designed such that the glue layer may be removable and replaceable. As such, the bandage would further include necessary components to enable charging of the battery disposed within the bandage.

In another embodiment, not shown, the bandage may include at least one circuit board disposed within the bandage. As described above, by moving the circuit board from the housing to the bandage, the size of the housing can be reduced. Alternatively, the circuit board disposed within the bandage may be utilized to enable the therapy providing device to be powered on. It shall be understood that the bandage may be manufactured to include both the energy source as well as the circuit board or any combination thereof.

The bandages disclosed herein may be fabricated from die-cutting, stamping, cutting, laser cutting, water jet cutting or other known manufacturing methods.

The bandages shown and described herein may be constructed using a die cutting process. If the die cutting process is used, then the bandage may be manufactured according to the following process.

Step 1, apply a sheet of the glue support layer to the die;

Step 2, apply force to the die and glue support layer, to form the insert apertures for the insert as well as create alignment apertures in sheet of the glue support layer to enable further die cutting processes;

Optional step 3, align sheet of glue support layer onto a set of alignment pins using the alignment apertures onto a die to form additional apertures in glue support layer to aid in flexibility and breathability;

Step 4, apply force to the die and the glue support layer to form the additional apertures;

Step 5, remove the glue support layer from the die;

Step 6, apply the cover layer to the glue support layer sheet;

Step 7, align the glue support layer/cover layer sheet with a die using the alignment apertures to cut an aperture in foam and cover layer;

Step 8, apply force to the die and glue support/cover layer sheet to form the aperture, remove the support/cover layer sheet from the die;

Step 9, disposed metallic inserts/magnets within the insert apertures formed in Step 2;

Step 10, apply the glue layer to the glue support layer;

Step 11, position the cover/glue support and glue layer within a die using the alignment apertures;

Step 12 apply force to cut through all layers of the bandage and to cut the outer shape of bandage; and

Step 13 package the completed bandage.

In yet another aspect of the disclosure, as described herein the magnets, which may be positioned in the device housing, the bandage or both, may be utilized to control the function of the therapy providing device 200. In this example, at least one of the magnets can be used as a switch to control or complete a power circuit. The power circuit can be activated such as to power the therapy providing device 200 on, thereby initiating therapy. If the magnetic connection is broken or disrupted, then the therapy providing device would be powered off. In this embodiment, the magnets within the therapy providing device may be coupled together with a conductive element or conductively coupled to a circuit board. Additionally, the bandage can include a coupling between the magnets/metallic members. The coupling on the bandage may be a wire connected between the elements, or a metallic strip in which the magnets/metallic members are in contact with. Further still, the magnets/metallic members may be coupled together through the use of a conductive ink/paint, which is disposed on a layer of the bandage, thereby electrically coupling the magnets/metallic members. The connection between the magnet/metallic members could be measured to be a voltage, impedance, resistance, magnetic gauss or other measurable parameter. The program executed by the processor can utilize this measurement to power on/off the therapy providing device. It is further contemplated, that in addition to the above, the magnet/metallic member within the device or bandage may be manufactured with specific properties, such that the therapy providing device will only operate with original equipment manufacturing products, thereby preventing the therapy providing device 200 from being utilized with non-approved or counterfeit bandages. For example, the magnet may be manufactured with specific magnetic field properties, these properties would be compared to a programmed property, if the properties match, then the device would be allowed to be turned on, if the properties do not match, then the device would not be able to be powered on, whereby an error code may be displayed on the LEDs or spoken through the driver assembly. A benefit of utilizing the magnets to switch the device on/off is that the user does not have to activate any buttons on the device, additionally, the device can be simplified through the elimination of the button on the therapy providing device as described herein. Another benefit is the preservation of battery life of the device, as the device will be powered off as soon as the magnetic connection is broken. Additionally, if the therapy providing device is being utilized at night time during sleep and the device becomes dislodged from the user, the device will automatically power off, thus providing an additional safety feature. It is further contemplated, that the therapy providing device when turned on, can record the time at which the therapy providing device was activated and store this information in memory, wherein, the therapy providing device may communicate such data to another location, such as wirelessly to the charging station, or a computing device or directly to a webpage/site.

It is further contemplated that a magnetic switch may be incorporated into the therapy providing device, wherein the switch would be moved to a closed or open position when the therapy providing device is brought into close proximity with a bandage containing a magnetic member, thereby allowing the therapy device to be powered on or to be automatically powered on. Further still, the magnetic switch may be utilized during charging of the therapy providing device. As described herein, a separate charging base may be provided, as previously described, to charge the energy source within the therapy providing device. It is contemplated that the charging station may include at least one magnet to align the therapy providing device with a portion of the charging station, wherein the magnetic switch may be activated by the magnet of the charging station, whereby the energy source of the therapy providing device can then be charged. Furthermore, as described above, the charging station may include at least two magnets, wherein the magnets are coupled to each other such that a signal can pass between them, the signal can be voltage, impedance, resistance and the like. As such, when the therapy providing device is placed onto the charging station, the energy source of the therapy providing device will only be charged after a program of the therapy providing device confirms a connection of the magnets of the charging station and the magnets of the therapy providing device. As used herein and throughout the application, it shall be understood that the magnets may be replaced with metallic members or other types of materials which may exhibit magnetic properties or may be attracted to magnets, or alternatively, mechanical fasteners such as a snap can be utilized.

Further still, as shown in FIG. 16G, the bandage may include pads 2450 and 2451 disposed on either side of the aperture 2431, the pads 2450 and 2451 can be coupled together and the pads 2450 and 2451 can also be coupled together. The pads may be coupled together either through a wired connection or through the use of another type of conductive material such as conductive ink, foil tape, or similar items as shown as elements 2436 and 2438. As described above, the charging pins of the housing are configured to couple, the charging pins/pads in this configuration can not only be used for charging as described above, but they may also be used in combination with the pads on the bandage as shown in FIG. 16G to determine when the therapy providing device 200 is attached to the bandage, which then may cause the therapy providing device 200 to be energized. Alternatively, the combination of the charging pins/pads

Further still, it is contemplated, that the therapy providing device 200 and/or bandage may include a security feature, such as an optical scanner disposed within the therapy providing device, such that the optical scanner is configured to scan a QR code, bar code or other coded printed on the bandage or bandage packaging. As described above, this combination of a scanner and specific code can be utilized to control the activation of the therapy providing device 200. Additionally, the use of a security code/barcode can be combined with the magnetic activation of the therapy providing device 200 as described above to ensure that the bandage being utilized is an approved product that has been designed to be specifically utilized with the therapy providing device 200 and that the bandage is not a third-party un-approved product or a counterfeit product.

It is contemplated that other types of security systems can be utilized to achieve the same or similar functions. For example, the bandage may include a protrusion (not shown) that projects above the surface of the bandage, the protrusion would be received within an aperture of the second surface of the housing where it would activate a switch within the housing. Another example would be the use of an electronic circuit or chip disposed upon or within the bandage, the circuit or chip would interface with the therapy providing device, thereby completing a circuit to enable activation of the therapy providing device.

The bandage may further include a metallic magnetic strip, similar to a credit card, whereby the therapy providing device or base station 570 may include a strip reader. In use the bandage(s) would be passed through the strip reader in order to activate the therapy providing device. The strip may include embedded information such as a serial number, prescribed therapy information, patient information or may include a security code that is utilized to unlock the therapy providing device or base station. Additionally, information may be collected each time the bandage is swiped to provide information to a physician or payer/payor to determine usage of the therapy providing device(s).

Additionally, the bandage and therapy device may contain RFID components, wherein when brought together the therapy device would be activated. It is contemplated that the therapy providing device may be “coded” to work with specific bandages, similar to the manner in which glucose meters are “coded” to each package of test strips. In this embodiment, a “code bandage” would be provided, wherein the therapy providing device would include a reader to read the code bandage, such that the code bandage would cause a change in the program of the therapy providing device. The code bandage may include a bar code, QR code, RFID component, a computer chip, a series of raised protrusions or similar designs to transmit a code to the therapy providing device. In each of the embodiments above the therapy providing device 200 would include an appropriate reader. It may be preferred to provide a “code bandage” with each package of bandages to be utilized. Alternatively, the therapy providing device may be provided with a certain number of bandages in a package, whereby when the bandages are expended the therapy providing device will cease to turn on until a new package of bandages is purchased, wherein the new package of bandages includes a code bandage to re-activate the therapy providing device. As described above, the base station 570 may include a QR or barcode reader assembly, wherein prior to use of the therapy providing system 100 of the present disclosure, according to some embodiments, the user would scan the bandages and/or the therapy providing devices prior to use. The scanning process may be utilized to control the activation of the therapy providing device or control the activation of the therapy providing device.

In accordance with the disclosure, the bandage may include a temperature sensitive device, such that when the bandage is placed onto the user, the temperature sensitive device would provide a visual feedback of the user's temperature. The feedback may be in the form of a color display or an LED/LCD readout or show up as a graph or timeline chart. Additionally, it is contemplated that the therapy providing device 200 may include and optical reader, wherein the optical reader may be configured to “read” the temperature display. By reading the temperature off of the bandage, the therapy providing device 200 may be turned off if the temperature range read is outside of a pre-determined range. Such range may be between 85 and 105 degrees Fahrenheit. The reading of the temperature may also be utilize to determine if the user has a fever, such fever may be an indication of a medical problem such as a hypertensive crisis or Cushing's Syndrome or other medical problem. Furthermore, by limiting operating window of the therapy providing device to a temperature range prevents the device from being activated until placed onto a user. For example, if the temperature range is 85 to 105 degrees Fahrenheit, then the device could not be activated when not disposed on the user, as such the user would not be able to “fake” therapy. This may be important in some embodiments, such as cases where insurance would be paying for usage of the therapy providing device.

It is further contemplated that the charging/base station 570 may include a bar code reader, RFID, ZIGBEE or other means which can be utilized to scan the code bandage. Additionally, it is contemplated that the base station 570 may be necessary to utilize the therapy providing device, wherein prior to use the therapy providing devices can be placed into/onto or nearby the base station in order to be activated. Additionally, it is contemplated that the bandages may also be placed in contact with, near or be scanned by the base station in order for the therapy providing devices to be activated. In another embodiment the computing device 300 may be incorporated into the base station 570, wherein the therapy cycle may be controlled by the base station 570.

In yet another embodiment, the therapy providing device may include at least one pressure sensor or flex sensor disposed adjacent the second surface of the housing, such that when the therapy providing device is coupled to a bandage on a user's skin, the pressure sensor or flex sensor would provide data indicating that the therapy providing device is placed onto a user's body. At this time, the therapy providing device may then be powered on to deliver therapy. The device may be powered on manually, automatically in response to the pressure sensor/flex sensor data or in response to a programmed parameter. The pressure/flex sensor(s) can provide an impedance/resistance data point which can be utilized by the program to determine the position of the therapy providing device. For example, the program could compare the resistance from the sensor against a look up table to determine if the therapy providing device is disposed on a user, disposed on the charging station and determine if the therapy device should be powered on or if charging should be enabled. Alternatively, the pressure sensor or flex sensor could be integrated into the bandage, whereby a conductive pad/pin arrangement would allow data from the pressure/flex sensor to be transferred to the therapy providing device.

As indicated above, the therapy providing device may be embodied in a kit form, wherein a pair of therapy providing devices would be provided along with a certain number of bandages. The bandages supplied may be for 1 week, 2 weeks, 3 weeks, 1 month, 2 months, 3 months, 4 months, 5 months, 6 months, 7 months, 8, months, 9, months, 10 months, 11 months or 1 year of therapy. As such it is contemplated that at least two bandages will be utilized for each therapy session, however, depending upon the schedule of therapy, up to four bandages may be utilized for each therapy session. As such, packages of bandages having a certain number of bandages can be provided depending upon the intended or prescribed therapy routine.

It is further contemplated that the metallic members/magnets as described herein can be replaced with other removable/detachable assemblies such as snap connectors, Velcro, twist-lock connectors or other similar connectors.

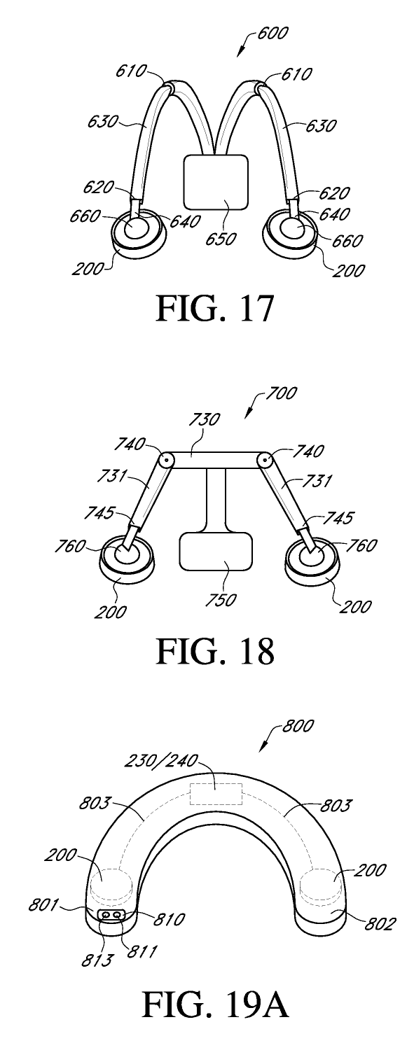

Referring now to FIG. 17 there is shown an alternative design for a mounting device to be utilized with the therapy system 100 in accordance with the present disclosure, according to some embodiments. As shown in FIG. 17 there is shown a support structure 600. The support structure 600 can be configured to position therapy providing devices 200 according to the present disclosure, according to some embodiments in a preferred location over a user's clavicle. The user can also adjust the positioning of the location of the therapy providing devices 200 by adjusting both the angle of the arm about pivot 610 and buy adjusting the length through the telescoping assembly 620.

The support structure 600 further includes a ball and cup joint 660 at the distal ends 640 of the arms 630. The ball and cup joint 660 is arranged to hold the therapy providing device 200 and allows a user to align the therapy providing device 200 substantially parallel to a surface of the user at the desired location to insure that as much as possible of the therapy providing device 200 is in contact with the user. It is contemplated that other types of flexible joints may be utilized in place of a ball and cup joint, for example a living hinge assembly or the like.

The support structure 600 further includes a pad 650 connected to the arms 630. In accordance with embodiments of the present disclosure, the pad may contain the electronics module 320 and the power source.

The arms 630 of the support structure 600 can also be configured to include a spring force to push the therapy providing device 200 against the body. For example, the arms 630 of the support structure 600 depicted in FIG. 17 are curved and are configured to apply a spring force between the therapy providing units 200 and the pad 650 when the support structure 600 is placed over a user's shoulders.

Referring now to FIG. 18 there is shown another example, of a support structure 700 in accordance with the present disclosure. As shown in FIG. 4H, the support structure 700 includes a pad 750, a first arm 730, and second arms 731. The support structure 700 further includes joints 740, the joints 740 join the first arm 730 to the second arms 731. The joints 740 are configured to allow for rotational motion between the first arm 730 and the second arms 731 in order to allow a user to align the therapy providing devices 200 in accordance with the methods of the present disclosure, according to some embodiments. The second arms 731 further include telescoping sections 745. The telescoping sections 745 allow the user to adjust the length of the second arms 731 to position the therapy providing units properly. The second arms 731 further include a ball joint assembly 760 disposed at their distal ends, the ball joint assemblies 760 couple the therapy providing units to the second arms 731. The ball joint assemblies 760 allow the therapy providing units to lay flat against the user's collar bones and account for differences in anatomy. The support structure 700 further includes a pad 750 coupled to the first arm 730. As described above the pad 750 may contain the electronics module 540 and the energy source 240. In certain embodiments the electronics module 540 and energy source 240 would be user replaceable. In other embodiments, the electronics module 540 and energy source 240 would not be user replaceable and the entire assembly would be replaced including the therapy providing devices.

The support structures 600 and 700 can be made of an elastic material. The elasticity of the design provides for a spring or clamping force, such that the support structure and therapy providing devices remain in position during use.

The support structures described herein can be configured to fit snugly without being too compressive on the body, are straightforward to put on over the shoulders or around the torso, and can be worn underneath clothing without significantly altering the profile of the clothing.

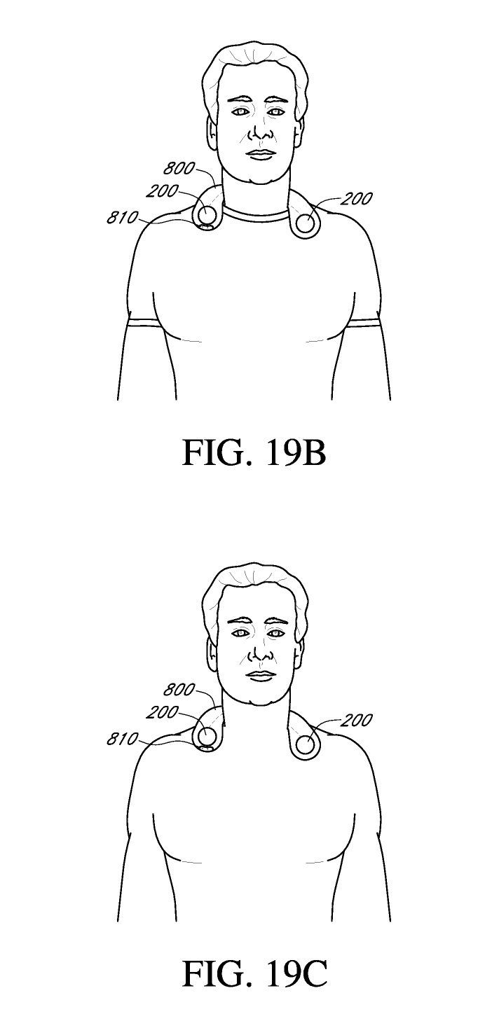

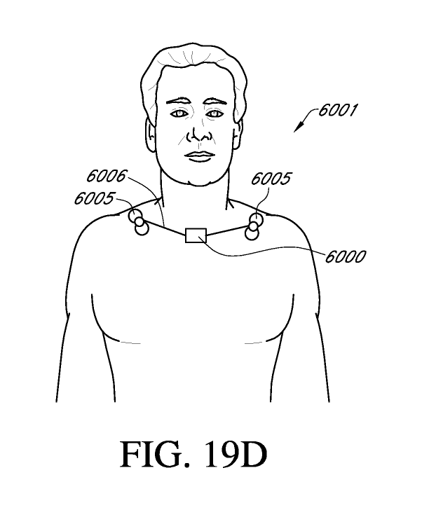

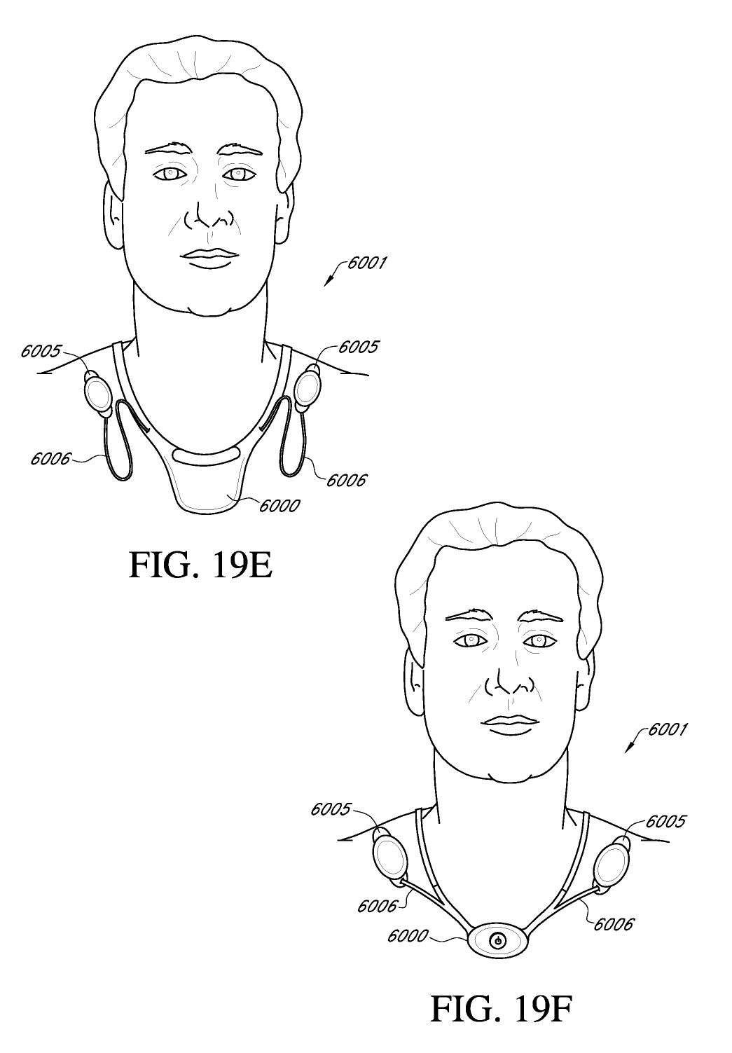

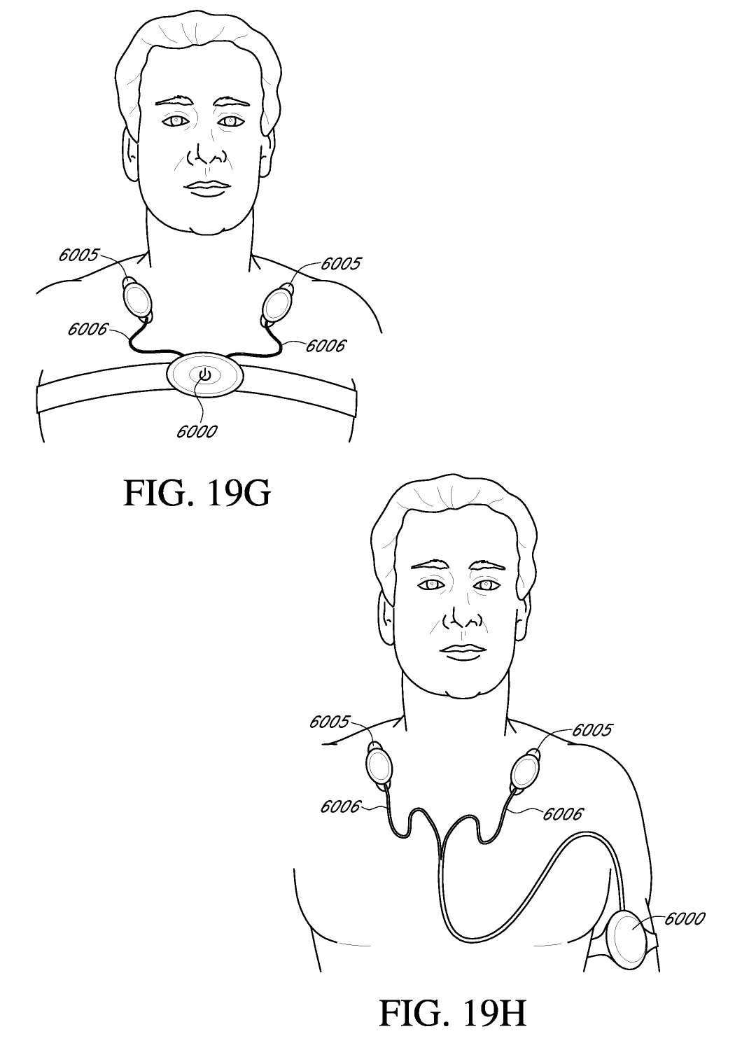

Referring now to FIGS. 19A-19C, there are shown additional embodiments of the present disclosure. As shown in FIGS. 19A-19C, the therapy providing device 200 of the present disclosure, according to some embodiments may be incorporated into a support structure 800, wherein the support structure 800 includes a proximal end 802 and a distal end 801 and an elongate member 803 extending between the two ends. The support structure further includes a control panel 810, wherein the control panel 810 may include an indicator such as a light or LED 811 to indicate the function of the therapy providing devices 200. The control panel 810 further includes a switch 813, the switch 813 being in electrical communication with the therapy providing devices 200 and the electronics module 540 and the energy source 240, each of which have been described above. The support structure 800 may be fabricated of fabric such as cotton, nylon, polyester or the like, wherein the body 803 is in the form of a tubular, square, rectangular or cylindrical shape, thereby forming an inner chamber. As shown in FIG. 4I, the therapy providing devices 200 and the electronics module 540 and energy source 240 are shown disposed within the inner chamber. These components may be held within the inner chamber through the use of pockets formed within the inner chamber. It is further contemplated that the inner chamber may be filled with a material to increase the weight of the overall device. Examples of materials that can be utilized to fill the chamber are rice, beans, sand, metallic materials, polymer materials, ceramic materials, and other such materials that are known to one skilled in the art.

As shown in FIGS. 19B and 19C, the support structure 800 can be worn around a user's neck and shoulders, wherein the therapy providing device 200 would be adjusted by the user to fall onto and make contact with the user's clavicle. The support structure 800 may be disposed over the top of a user's clothing as shown in FIG. 19B, or alternatively the support structure 800 may be disposed directly against a user's skin as shown in FIG. 19C.

In accordance with the embodiment shown in FIGS. 19A-19C, it is contemplated that the support structure may further include a removable cover (not shown), wherein the removable cover can be disposed about the support structure 800. The removable cover may include a zipper, velcro or snaps to open and close the cover. Additionally, the removable cover may include additional items such as pads placed along a portion or a length thereof. For example, a pad may be disposed on the cover near the user's neck area.

It is further contemplated that the support structure 800 in accordance with the present disclosure, according to some embodiments may include additional features. For example, a heating element may be incorporated into the support structure 800, whereby the heating element may be utilized by the user to address sore muscles or neck pain.

Referring now to FIGS. 19D-19H there are shown other embodiments of the therapy providing system 100 in accordance with the disclosure. As shown in FIG. 19D, there is shown an alternative construction of the therapy providing system of the present disclosure, according to some embodiments. As shown in FIG. 19D, the therapy providing system 6001 includes a pair of housings 6005 and a central housing 6000. In this embodiment, it is contemplated that the housings 6005 contain the driver assembly 220 as described herein above. The central housing 6000 may then contain the other components such as at least one circuit board and an energy source. The driver assemblies 220 contained in the housings 6005 include a cable connection 6006 coupling the driver assemblies 220 to the components disposed within the central housing 6000.

Referring now to FIGS. 19E and 19F, there is shown an alternative construction of the therapy providing system of the present disclosure, according to some embodiments. As shown in FIG. 19D, the therapy providing system 6001 includes a pair of housings 6005 and a central housing 6000. In this embodiment, it is contemplated that the housings 6005 contain the driver assembly 220 as described herein above. The central housing 6000 may then contain the other components such as at least one circuit board and an energy source. The driver assemblies 220 contained in the housings 6005 include a cable connection 6006 coupling the driver assemblies 220 to the components disposed within the central housing 6000. As shown in FIGS. 19E and 19F, the central housing 6000 is configured to be disposed about the patients neck.

Referring now to FIG. 19G there is shown an alternative construction of the therapy providing system of the present disclosure, according to some embodiments. As shown in FIG. 19D, the therapy providing system 6001 includes a pair of housings 6005 and a central housing 6000. In this embodiment, it is contemplated that the housings 6005 contain the driver assembly 220 as described herein above. The central housing 6000 may then contain the other components such as at least one circuit board and an energy source. The driver assemblies 220 contained in the housings 6005 include a cable connection 6006 coupling the driver assemblies 220 to the components disposed within the central housing 6000. As shown in FIG. 19G, the central housing 6000 is configured to be strapped about a patient's chest in a similar manner to a heart rate monitor. In this embodiment, the central housing and strap and also be configured to function as a heart rate monitor or an EKG monitor.

Referring now to FIG. 19H there is shown an alternative construction of the therapy providing system of the present disclosure, according to some embodiments. As shown in FIG. 19D, the therapy providing system 6001 includes a pair of housings 6005 and a central housing 6000. In this embodiment, it is contemplated that the housings 6005 contain the driver assembly 220 as described herein above. The central housing 6000 may then contain the other components such as at least one circuit board and an energy source. The driver assemblies 220 contained in the housings 6005 include a cable connection 6006 coupling the driver assemblies 220 to the components disposed within the central housing 6000. In this embodiment the central housing 6000 is configured to be worn about a user's arm as shown using an arm band. It is further contemplated in this embodiment, the arm band may be utilized as a blood pressure cuff for a blood pressure monitor, wherein the central housing 6000 further includes the necessary electronics and mechanical components to function as a blood pressure monitor in addition to being a therapy providing device 200 in accordance with the present disclosure, according to some embodiments.

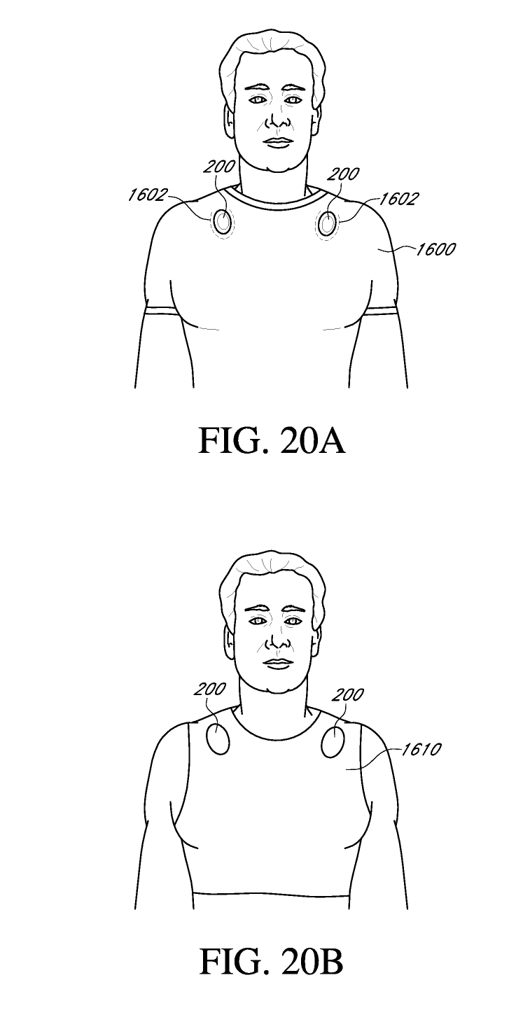

Referring now to FIGS. 20A and 20B, there are shown embodiments of clothing articles which can be utilized with the therapy providing device 200 of the present disclosure.

As shown in FIG. 20A, in one embodiment, the clothing article is embodied as a t-shirt 1600, wherein the t-shirt 1600 includes pockets 1602 formed therein to receive the therapy providing device 200. The pockets 1602 are aligned over the user's clavicle in order to provide treatment as will be described below.

Referring now to FIG. 20B there is shown an alternative embodiment of a piece of clothing configured to retain the therapy device 200 in accordance with the present disclosure. As shown in FIG. 20B the clothing can be embodied in the form of a sports bra 1610. The sports bra 1610 further includes pockets 1612 configured to receive the therapy device 200. Alternatively, instead of pockets, other attachment mechanisms such as those described above, wherein instead of pockets, magnets, hook and loop fasteners, snap fasteners, twist and lock or similar types of fastening systems may be utilized to retain the therapy providing device 200 in position.

Additionally, the clothing devices described above may further include an additional pocket or pockets to receive the computing device, or in embodiments wherein the electronics or energy source are separate from the therapy providing device, pockets or other retention means to retain these additional components.

The clothing devices may further include a structure formed therein or attached thereto (not shown) wherein the structure is configured to apply a downward force upon the therapy providing device(s). Structures similar to those shown in FIGS. 17, 18 and 19 may be utilized.

In accordance with the present disclosure, according to some embodiments, the therapy providing device 200 may include addition features. One such additional feature can be the inclusion of a thermometer to track the user's temperature during use and/or the ambient room temperature. Another additional feature can be the inclusion of a sleep sensor or sleep tracking program, wherein the therapy providing device can be utilized to track the user's sleep. For example, the sleep program may utilize the GPS/accelerometer of the therapy providing device to track movement during sleep, wherein the sleep program could further utilize the temperature data as well. Another aspect of the disclosure could be to utilize the therapy providing device to be further utilized to diagnose sleep apnea, wherein the therapy providing device could further include a microphone to enable audio recording of the user's breathing and or snoring during sleep. Additionally, the microphone recording of the breathing can be combined with the accelerometer data or GPS/tilt data to correlate the breathing recordings to the specific user. Further still, the microphone could be utilized to turn the therapy providing device on/off. In this embodiment, the computer readable program may be configured to respond to voice commands, such that a user could power the therapy providing device on/off with specific voice commands.

In yet another embodiment, the microphone, coupled with the processor can be utilized to detect specific sounds, which when detected will power the therapy providing device on/off. For example, the microphone may be configured to detect a user's heartbeat, thus, based on the detection of heart beat the therapy providing device may be powered on/off. The microphone could also be utilized to detect and determine a user's heart rate, as such, the device could be powered on/off based on a pre-set heart rate parameter. For example, if the user's heart rate is outside of a pre-set parameter, such as 180 BPM (beats per minute) the device may be powered off for safety.

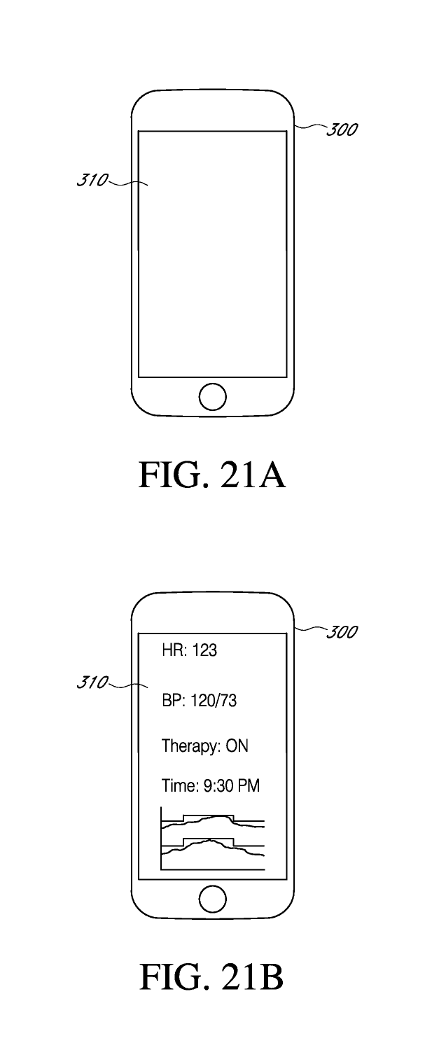

Referring now to FIGS. 21A and 21B there is shown a computing device 300 in accordance with the present disclosure, according to some embodiments. The computing device 300 includes a processor, memory, energy source (such as a battery), and a display 310. The computing device may be a custom manufactured device for use with the therapy device 200 as described above, or alternatively, the computing device 300 may be a commercially available device such as a smartphone or tablet. Examples of such commercially available devices are iOS enabled devices such as the iPhone®, iPad®, iPod®, Android based phones and/or tablets, laptops or computers. As shown in FIG. 15B, the computing device may be configured to display a user's heart rate and blood pressure when connected to a therapy providing device having those measurement capabilities or where other compatible devices are utilized with the therapy providing device. Alternatively, the computing device may display data received from one or more therapy providing devices, this data may include start/stop times of therapy provided by the therapy providing device 200, battery status of one or more therapy providing devices and the like.

In accordance with the present disclosure, according to some embodiments, the computing device 300 is configured to run a program 820. In accordance with the present disclosure, according to some embodiments, the program 820 is configured to communicate with the therapy device 200. The communication between the program 820, computing device 300 and the therapy device 200 may be conducted using Bluetooth, WI-FI, ZIGBEE, NFC, RFID, ANT+, 3G/4G, cellular connection or other known wireless communication protocols. Alternatively, the computing device may be coupled to at least one of the therapy devices through a cable connection.

In an alternative embodiment, the program 820 is stored on memory located within the memory of the therapy providing device 200. The program maybe initiated manually through the use of a physical button pressed by the user. Alternatively, the program 820 may be initiated automatically by a timer located within the therapy providing device 200. The timer may further utilize data inputs from an accelerometer/compass or tilt sensor to indicate when the user is in a prone position to initiate the program 820. Further still, the timer may receive input from an impedance sensor indicating whether the therapy providing device 200 is in proper placement on the users body. The program would then be initiated based on the inputs received. The device may be activated further by the light sensor. For example, when the light sensor detects darkness, the therapy providing device can be powered on either manually or automatically. Alternatively, the light sensor may detect the user's skin wherein the therapy providing device may be powered on, the therapy providing device may be powered on manually, automatically or in response to the computer readable program, either from the darkness against the skin. The device may be activated from the reduced light from the user's surroundings, for example when the user is sleeping. Further still, the bandage may include a reflective portion, wherein the light sensor receives a reflectance from the reflective portion of the bandage to power the therapy providing device on/off.

In certain embodiments, the program 820 is pre-configured to deliver therapy using the therapy providing device through pre-programmed parameters. The HCP may adjust the therapy parameters within the program 820, such that the therapy provided to the user may be customized to the user. The customization of the therapy may be changes to the wavelength, amplitude, duration, start/stop times. The customization may be done by the HCP while providing services to the patient, for example, the HCP may apply the therapy providing device to the patient, initiate therapy and monitor the patient's response. Through this active monitoring, the HCP may change the parameters of the program to elicit a response in the patient. For example, it is contemplated that certain patients may have different bone densities; therefore the therapy provided by the device may need to be adjusted accordingly. It is further contemplated, that once programmed, the user cannot change the therapy parameters of the program, or alternatively, certain parameters or all parameters may be open to change by the end user or remotely. Alternatively, the HCP, after determining the best therapy parameters, can choose from multiple programs stored within memory of the therapy providing device. Further still, the HCP may be provided with a dedicated programming device, or may couple the device to a personal computer, smartphone, tablet or other internet enabled device, such that the HCP can utilize the dedicated programmer or download over a secure internet connection, programs to be uploaded into the therapy providing device.

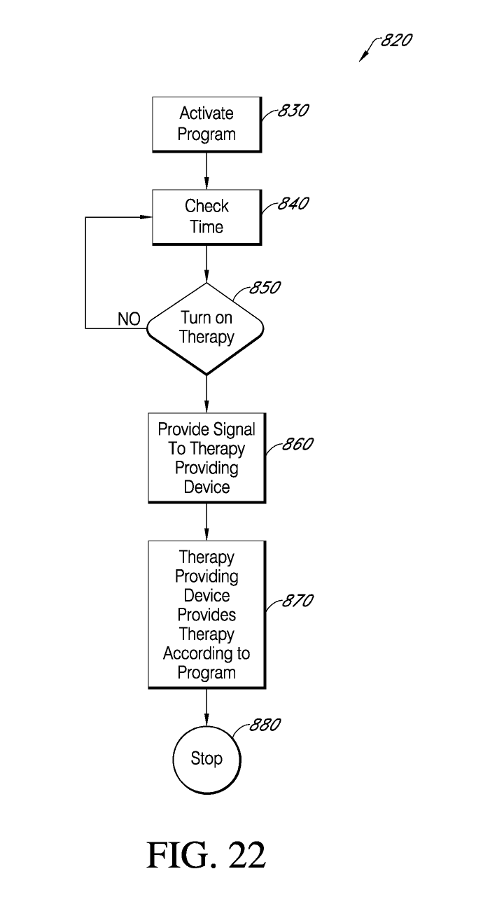

Referring now to FIG. 22, there is shown a flow diagram illustrating the program 820 in accordance with one embodiment of the present disclosure. As shown in FIG. 22, the program 820 may configured to be run on the computing device 300 to control the therapy applied to the user by the therapy providing device 200. Alternatively, it is contemplated that the program 820 may reside within memory within the therapy providing device 200 as described above.

At Box 830, the user activates the program on the computing device 300 or therapy providing device 200. Or the therapy providing device is automatically activated when placed on the user's body.

At Box 840, the program checks the time on the computing device 300 or internally from the clock circuit of the therapy providing device 200.

At Box 850, the program determines whether to turn the therapy providing device on based upon the time check in Box 840. If the time is before a pre-programmed time or a user set time, then the program returns to Box 840. If the time is after the pre-set time or user set time, then the program turns the therapy providing device on. In accordance with the disclosure, if the time is received from the computing device, a user may adjust the time of the computing device, for example if the computing device is moved from one time zone to another. Alternatively, the computing device may automatically update the time.

At Box 860, the therapy providing device 200 is provided with a signal generated by the program and transmitted from the computing device 300 through a selected transmission method. In alternative embodiments, the therapy providing device contains a processor and memory, wherein a program is retained within the memory of the therapy providing device. In this embodiment, the signal provided by the computing device 800, is a power on/off signal, wherein once powered on the program residing within the memory of the therapy providing device will begin to run.

At Box 870, the therapy device provides therapy to the patient. In the process of providing therapy, a signal is transmitted to the therapy device 200 by the computing device 300 through as directed by the program 820, or as described above, the program residing in the memory of the therapy providing device runs. In one embodiment, the therapy is applied for a set period of time. In alternative embodiments, the time duration of the therapy may be determined based upon data received from other sensors disposed upon the user or about the user. In yet another embodiment, the user may manually deactivate the therapy providing device/program.

At Box 880, the therapy is stopped. The therapy may be stopped based upon a time event, motion event, manually by the user, automatically by the program.

During each of the steps described above and shown in the flow diagram of FIG. 22, the user may be presented with displays on the screen 810 of the computing device. The screen 810 may display the start and stop times of the therapy, these times may be set by the user or may be set for the user by a health care provider. Alternatively, the times maybe automatically generated in response to data received from other sensors as will be described in detail below.

According to the disclosure, the program includes a non-transitory computer readable medium having computer executable program code embodied thereon, the computer executable program code configured to send appropriate signals to the circuit board(s) 500/550 to provide therapy in accordance with the methods of the present disclosure, according to some embodiments utilizing the therapy providing device 200 of the present disclosure, according to some embodiments.

### Methods of Use

In accordance with the present disclosure, according to some embodiments, methods of use will be described below. The methods described shall be considered to be exemplary and should not be considered limiting in any manner.

In accordance with one embodiment of the present disclosure, the therapy device includes a driver assembly, wherein the driver assembly is embodied as a speaker as shown in FIG. 5A and as described herein. The speaker may be a haptic speaker, a piezoelectric speaker, an electroactive polymeric transducer, or a magnetic coil speaker. The computing device 800 and program 810 are configured to provide a signal to the speaker to cause the speaker to vibrate at certain frequencies or to oscillate, translate, step, transition or sweep through a range of frequencies.

In accordance with embodiments of the present disclosure, the frequencies contemplated for use can range between 0 Hz to 20,000 Hz, 0 Hz and 10,000 Hz, 0 Hz and 5,000 Hz, 0 Hz and 2,500 Hz, 0 Hz and 1,750 Hz, 0 Hz and 875 Hz, 0 Hz and 435 Hz, 0 Hz and 200 Hz, 0 Hz and 150 Hz, 1 Hz and 150 Hz, 2 Hz and 150 Hz, 3 Hz and 150 Hz, 4 Hz and 150 Hz, 5 Hz and 150 Hz, 6 Hz and 150 Hz, 7 Hz and 150 Hz, 8 Hz and 150 Hz, 9 Hz and 150 Hz, 10 Hz and 150 Hz, 11 Hz and 150 Hz, 12 Hz and 150 Hz, 13 Hz and 150 Hz, 14 Hz and 150 Hz, 15 Hz and 150 Hz, 16 Hz and 150 Hz, 17 Hz and 150 Hz, 18 Hz and 150 Hz, 19 Hz and 150 Hz, 20 Hz and 150 Hz, 21 Hz and 150 Hz, 22 Hz and 150 Hz, 23 Hz and 150 Hz, 24 Hz and 150 Hz, 25 Hz and 150 Hz, 26 Hz and 150 Hz, 27 Hz and 150 Hz, 28 Hz and 150 Hz, 28 Hz and 150 Hz, 29 Hz and 150 Hz, 30 Hz and 150 Hz, 31 Hz and 150 Hz, 32 Hz and 150 Hz, 33 Hz and 150 Hz, 34 Hz and 150 Hz, 35 Hz and 150 Hz, 36 Hz and 150 Hz, 37 Hz and 150 Hz, 38 Hz and 150 Hz, 39 Hz and 150 Hz, 40 Hz and 150 Hz, 41 Hz and 150 Hz, 42 Hz and 150 Hz, 43 Hz and 150 Hz, 44 Hz and 150 Hz, 45 Hz and 150 Hz, 46 Hz and 150 Hz, 47 Hz and 150 Hz, 48 Hz and 150 Hz, 49 Hz and 150 Hz, 50 Hz and 150 Hz, 51 Hz and 150 Hz, 52 Hz and 150 Hz, 53 Hz and 150 Hz, 54 Hz and 150 Hz, 55 Hz and 150 Hz, 56 Hz and 150 Hz, 57 Hz and 150 Hz, 58 Hz and 150 Hz, 59 Hz and 150 Hz, 60 Hz and 150 Hz, 61 Hz and 150 Hz, 62 Hz and 150 Hz, 63 Hz and 150 Hz, 64 Hz and 150 Hz, 65 Hz and 150 Hz, 66 Hz and 150 Hz, 67 Hz and 150 Hz, 68 Hz and 150 Hz, 69 Hz and 150 Hz, 70 Hz and 150 Hz, 71 Hz and 150 Hz, 72 Hz and 150 Hz, 73 Hz and 150 Hz, 74 Hz and 150 Hz, 75 Hz and 150 Hz, 76 Hz and 150 Hz, 77 Hz and 150 Hz, 78 Hz and 150 Hz, 79 Hz and 150 Hz, 80 Hz and 150 Hz, 81 Hz and 150 Hz, 82 Hz and 150 Hz, 83 Hz and 150 Hz, 84 Hz and 150 Hz, 85 Hz and 150 Hz, 86 Hz and 150 Hz, 87 Hz and 150 Hz, 88 Hz and 150 Hz, 89 Hz and 150 Hz, 90 Hz and 150 Hz, 91 Hz and 150 Hz, 92 Hz and 150 Hz, 93 Hz and 150 Hz, 94 Hz and 150 Hz, 95 Hz and 150 Hz, 96 Hz and 150 Hz, 97 Hz and 150 Hz, 98 Hz and 150 Hz, 99 Hz and 150 Hz, 100 Hz and 150 Hz, 101 Hz and 150 Hz, 102 Hz and 150 Hz, 103 Hz and 150 Hz, 104 Hz and 150 Hz, 105 Hz and 150 Hz, 106 Hz and 150 Hz, 107 Hz and 150 Hz, 108 Hz and 150 Hz, 109 Hz and 150 Hz, 110 Hz and 150 Hz, 111 Hz and 150 Hz, 112 Hz and 150 Hz, 113 Hz and 150 Hz, 114 Hz and 150 Hz, 115 Hz and 150 Hz, 116 Hz and 150 Hz, 117 Hz and 150 Hz, 118 Hz and 150 Hz, 119 Hz and 150 Hz, 120 Hz and 150 Hz, 121 Hz and 150 Hz, 122 Hz and 150 Hz, 123 Hz and 150 Hz, 124 Hz and 150 Hz, 125 Hz and 150 Hz, 126 Hz and 150 Hz, 127 Hz and 150 Hz, 128 Hz and 150 Hz, 129 Hz and 150 Hz, 130 Hz and 150 Hz, 131 Hz and 150 Hz, 132 Hz and 150 Hz, 133 Hz and 150 Hz, 134 Hz and 150 Hz, 135 Hz and 150 Hz, 136 Hz and 150 Hz, 137 Hz and 150 Hz, 138 Hz and 150 Hz, 139 Hz and 150 Hz, 140 Hz and 150 Hz, 141 Hz and 150 Hz, 142 Hz and 150 Hz, 143 Hz and 150 Hz, 144 Hz and 150 Hz, 145 Hz and 150 Hz, 146 Hz and 150 Hz, 147 Hz and 150 Hz, 148 Hz and 150 Hz, 149 Hz and 150 Hz, 150 Hz and 150 Hz, 60 Hz and 100 Hz, 61 Hz and 100 Hz, 62 Hz and 100 Hz, 63 Hz and 100 Hz, 64 Hz and 100 Hz, 65 Hz and 100 Hz, 66 Hz and 100 Hz, 67 Hz and 100 Hz, 68 Hz and 100 Hz 69 Hz and 100 Hz, 70 Hz and 100 Hz, 60 Hz and 99 Hz, 61 Hz and 99 Hz, 62 Hz and 99 Hz, 63 Hz and 99 Hz, 64 Hz and 99 Hz, 65 Hz and 99 Hz, 66 Hz and 99 Hz 67 Hz and 99 Hz, 68 Hz and 99 Hz, 69 Hz and 99 Hz and 70 Hz and 99 Hz, and 61 Hz and 98 Hz, 62 Hz and 98 Hz, 63 Hz and 98 Hz, 64 Hz and 98 Hz, 65 Hz and 98 Hz, 66 Hz and 98 Hz, 67 Hz and 98 Hz, 68 Hz and 98 Hz, 69 Hz and 98 Hz and 70 Hz and 98 Hz.

In a preferred embodiment the signal causes the driver assembly to vibrate at a frequency or sweep through a range of frequencies between about 40 Hz and 150 Hz, more preferably between 50 Hz and 125 Hz, most preferably between about 60 Hz and 115 Hz. In accordance with the present disclosure, according to some embodiments, a therapeutic response has been achieved utilizing a frequency range between 65 Hz and 100 Hz.

It is further contemplated that these frequencies may be doubled and still achieve the therapeutic lowering of blood pressure in accordance with the present disclosure, according to some embodiments. It is further contemplated that these frequencies may be halved and still achieve the therapeutic lowering of blood pressure in accordance with the present disclosure, according to some embodiments.

In additional embodiment of the present disclosure, the driver assembly may vibrate or sweep or step or transition between frequencies of between: 60 Hz, 61 Hz, 62 Hz, 63 Hz, 64 Hz, 65 Hz, 66 Hz, 67 Hz, 68 Hz, 69 Hz, 70 Hz, 71 Hz, 72 Hz, 73 Hz, 74 Hz, 75 Hz, 76 Hz, 77 Hz, 78 Hz, 79 Hz, 80 Hz, 81 Hz, 82 Hz, 83 Hz, 84 Hz, 85 Hz, 86 Hz, 87 Hz, 88 Hz, 89 Hz, 90 Hz, 91 Hz, 92 Hz, 93 Hz, 94 Hz, 95 Hz, 96 Hz, 97 Hz, 98 Hz, 99 Hz and 100 Hz.

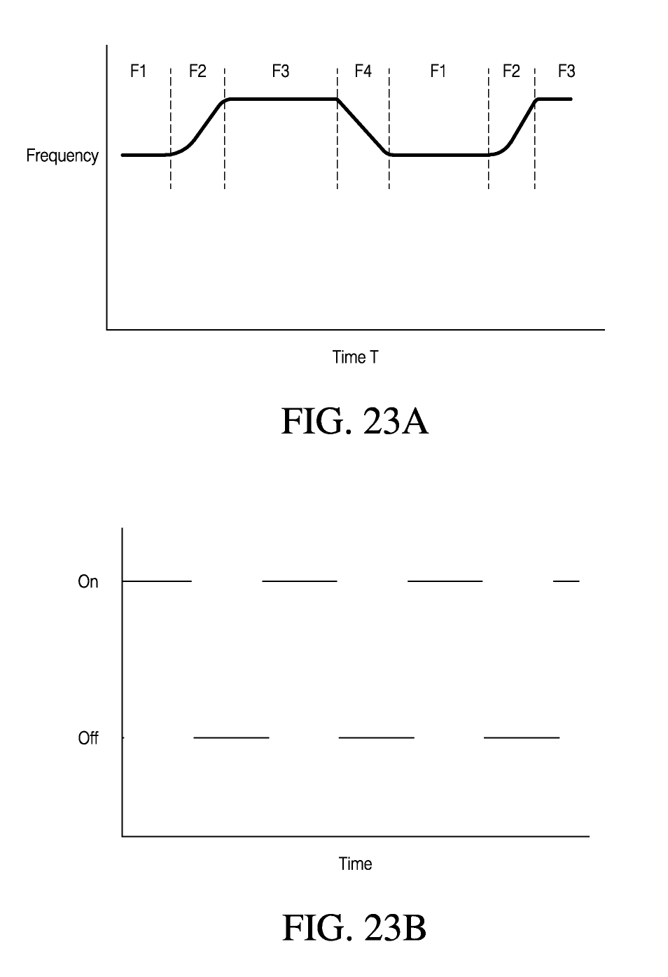

Referring now to FIGS. 23A and 23B, there is shown a further embodiment wherein the therapy is provided utilizing a combination of single frequencies and a sweeping frequency. For example, the driver assembly would be driven to vibrate at a single frequency, F1, for a period of time, then driven to sweep through a range of frequencies, F2, then driven at a single frequency, F3, different than the first single frequency, F1, and then finally driven backwards through the sweep of frequencies above, F4. This cycle may be repeated for a set period of time, turned off for a period of time and then repeated again, until an overall time period of therapy is reached.

Other contemplated frequencies in accordance with the present disclosure, according to some embodiments are: from 65.4 Hz to 98 Hz on one therapy providing device and from 65.4 Hz to 98 Hz on the other device, such frequencies result in a Binaural frequency of zero between the devices. Another pair of frequencies are from 65.4 Hz to 96 Hz on one therapy providing device and from 65.4 Hz to 98 Hz on the other device, such frequencies result in a Binaural frequency of 1 to 2 Hz on between the devices. Another pair of frequencies are from 40 Hz to 98 Hz on one therapy providing device and from 40 Hz to 98 Hz on the other device, such frequencies result in a Binaural frequency of zero between the devices. Another pair of frequencies are from 40 Hz to 80 Hz on one therapy providing device and from 40 Hz to 80 Hz on the other device, such frequencies result in a Binaural frequency of zero between the devices. And lastly, another pair of frequencies are from 40 Hz to 79 Hz on one therapy providing device and from 41 Hz to 81 Hz on the other device, such frequencies result in a Binaural frequency of between 1 and 2 Hz between the devices.

Additionally it is contemplated that in use, wherein two therapy provided devices are utilized, the frequencies provided by each of the two therapy providing devices may have different slopes. Such that as the therapy providing device(s) transition through the frequencies, one of the device may move between the frequencies faster or slower than the other device.

It is further contemplated that multiple signals utilizing separate frequencies may be transmitted by the program to the speaker. For example, one signal may be transmitted at one frequency and a second signal at another frequency. The signals may be transmitted simultaneously, independently or in an alternating fashion. If at least two therapy providing devices 200 are utilized, then one therapy providing device 200 may receive a first signal and the other receives a second signal.

In one embodiment, at least two therapy providing devices 200 are utilized. In use a signal will be sent to one of the two therapy providing devices 200, causing the speaker to emit a signal having a chosen frequency or range of frequencies. The signal is transmitted to a first therapy providing device 200 for a pre-determined period of time. After such time, the signal is terminated. Upon termination of the first signal, a second signal is generated and transmitted to the other therapy providing device. This second signal causes the speaker to emit a signal having a chosen frequency or range or frequencies. The chosen frequency may be the same as that transmitted to the first therapy providing device or it may be at a different frequency. The second signal will be transmitted to the second therapy providing device for a pre-determined period of time. After such time, the signal is terminated. The program will continued to run, however, during this time no signal will be transmitted to either therapy providing device 200, thereby creating a pause between activation of the therapy providing devices 200. After the pre-determined time period of the pause has passed, the program will then enter a loop and repeat the process described above. This pattern of therapy will repeat for as long as the program has been instructed to do so.

In the embodiment where two therapy providing devices 200 are utilized, each of the devices deliver a waveform to the user's left and right clavicle. The waveform is transmitted from the speaker in each of the therapy providing devices to the user's clavicles. The waveform is transmitted through the clavicle on the left and right side, where both waves meet at the sternum to create a standing wave.

Further still, in accordance with the present disclosure, according to some embodiments, the amplitude of the signal can be adjusted to adjust the sound pressure generated by the driver assembly 220 of the therapy providing device 200. It is contemplated that the amplitude may be doubled or increased even more to deliver the therapy in accordance with the present disclosure, according to some embodiments. In accordance with the disclosure, the therapy providing device 200 may be configured to provide a sound pressure between: 0 to 150 decibels, 0 to 100 decibels, 0 to 99 decibels, 0 to 98 decibels, 0 to 97 decibels, 0 to 96 decibels, 0 to 95 decibels, 0 to 94 decibels, 0 to 93 decibels, 0 to 92 decibels, 0 to 91 decibels, 0 to 90 decibels, 0 to 89 decibels, 0 to 88 decibels, 0 to 87 decibels, 0 to 86 decibels, 0 to 85 decibels, 0 to 84 decibels, 0 to 83 decibels, 0 to 82 decibels, 0 to 81 decibels, 0 to 80 decibels, 0 to 79 decibels, 0 to 78 decibels, 0 to 77 decibels, 0 to 76 decibels, 0 to 75 decibels, 0 to 74 decibels, 0 to 73 decibels, 0 to 72 decibels, 0 to 71 decibels, 0 to 70 decibels, 0 to 69 decibels, 0 to 68 decibels, 0 to 67 decibels, 0 to 66 decibels, 0 to 65 decibels, 0 to 64 decibels, 0 to 63 decibels, 0 to 62 decibels, 0 to 61 decibels, 0 to 60 decibels, 0 to 59 decibels, 0 to 58 decibels, 0 to 57 decibels, 0 to 56 decibels, 0 to 55 decibels, 0 to 54 decibels, 0 to 53 decibels, 0 to 52 decibels, 0 to 51 decibels, 0 to 50 decibels, 0 to 49 decibels, 0 to 48 decibels, 0 to 47 decibels, 0 to 46 decibels, 0 to 45 decibels, 0 to 44 decibels, 0 to 43 decibels, 0 to 42 decibels, 0 to 41 decibels, 0 to 40 decibels, 0 to 39 decibels, 0 to 38 decibels, 0 to 37 decibels, 0 to 36 decibels, 0 to 35 decibels, 0 to 34 decibels, 0 to 33 decibels, 0 to 32 decibels, 0 to 31 decibels, 0 to 30 decibels, 0 to 29 decibels, 0 to 28 decibels, 0 to 27 decibels, 0 to 26 decibels, 0 to 25 decibels, 0 to 24 decibels, 0 to 23 decibels, 0 to 22 decibels, 0 to 21 decibels, 0 to 20 decibels, 0 to 19 decibels, 0 to 18 decibels, 0 to 17 decibels, 0 to 16 decibels, 0 to 15 decibels, 0 to 14 decibels, 0 to 13 decibels, 0 to 12 decibels, 0 to 11 decibels, 0 to 10 decibels, 0 to 9 decibels, 0 to 8 decibels, 0 to 7 decibels, 0 to 6 decibels, 0 to 5 decibels, 0 to 4 decibels, 0 to 3 decibels, 0 to 2 decibels, 0 to 1 decibels, 0 to 0.5 decibels, 0 to 0.25 decibels, 10 to 100 decibels, 20 to 100 decibels, 30 to 100 decibels, 40 to 100 decibels, 50 to 100 decibels, 60 to 100 decibels, 70 to 100 decibels, 80 to 100 decibels, 90 to 100 decibels, 10 to 75 decibels, 20 to 75 decibels, 30 to 75 decibels, 40 to 75 decibels, 50 to 75 decibels, 60 to 75 decibels, 70 to 75 decibels, 10 to 65 decibels, 20 to 65 decibels, 30 to 65 decibels, 40 to 65 decibels, 50 to 65 decibels and 60 to 65 decibels, 20 to 30 decibels, 30 to 40 decibels, 40 to 50 decibels, 50 to 60 decibels, 60 to 70 decibels, 70 to 75 decibels, 80 to 90 decibels, 50 to 75 decibels and 50 to 65 decibels.

Further still, in accordance with the present disclosure, according to some embodiments, the standing wave may be of half-octave, double octave, or reflective incidence. Thus the frequencies delivered at the collarbone may independently collide across the breastbone or sternum and create a new frequency which is of a different or same frequency as the generating waves.

In accordance with the present disclosure, according to some embodiments, the frequency selected for therapy may be held constant while the sound pressure level can be increased or decreased, alternatively, the sound pressure level may be held constant and the frequency varied. The measurement of a sound pressure level is related to the displacement of a portion of the driver 220. The portion of the driver 220 may be displaced between: 0 mm and 20 mm, 0 mm to 10 mm, 0 mm to 9 mm, 0 mm and 8 mm, 0 mm to 7 mm, 0 mm to 6 mm, 0 mm to 5 mm, 0 mm and 4 mm, 0 mm and 3 mm, 0 mm and 2 mm, 0 mm and 1 mm, 0 mm and 0.5 mm, 0 mm to 0.05 mm, 0 mm to 0.005 mm, 0 mm to 0.0005 mm, 0.5 mm to 0.05 mm, 0.5 mm to 0.005 mm, 0.05 mm to 0.005. If the driver 220 is selected to be the haptic speaker 220′, then the portion of the haptic speaker 220′ being displaced is the coil of the haptic speaker.

In accordance with the present disclosure, according to some embodiments, it is contemplated that each therapy providing device may be activated to provide therapy for a time period between about 1 second and 24 hours. In other embodiments, the therapy providing devices may be activated to provide therapy for a time period of between about 1 second and 12 hours, 1 second and 11 hours, 1 second and 10 hours, 1 second and 9 hours, 1 second and 8 hours, 1 second and 7 hours, 1 second and 6 hours, 1 second and 5 hours, 1 second and 4 hours, 1 second and 3 hours 1 second and 2 hours, and 1 second and 1 hour, 1 second and 45 minutes, 1 second and 30 minutes, 1 second and 20 minutes, 1 second and 15 minutes, 1 second and 10 minutes, 1 second and 5 minutes and 1 second and 1 minute.

The overall therapy process may be conducted for a time period between 1 second and 24 hours, 1 second and 23 hours, 1 second and 22 hours, 1 second and 21 hours, 1 second and 20 hours, 1 second and 19 hours, 1 second and 18 hours, 1 second and 17 hours, 1 second and 16 hours, 1 second and 15 hours, 1 second and 15 hours, 1 second and 14 hours, 1 second and 13 hours, 1 second and 12 hours, 1 second and 11 hours, 1 second and 10 hours, 1 second and 9 hours, 1 second and 8 hours, 1 second and 7 hours, 1 second and 6 hours, 1 second and 5 hours, 1 second and 4 hours, 1 second and 3 hours, 1 second and 2 hours, 1 second and 1 hour, 1 second and 45 minutes, 1 second and 30 minutes, 1 second and 15 minutes, 1 second and 10 minutes, 1 second and 5 minutes, 1 second and 1 minute.

In an alternative embodiment, instead of activating one therapy providing device 200 at a time to conduct the therapy, both therapy providing devices 200 may be activated at the same time.

In use, two therapy providing devices may be utilized, wherein the therapy providing devices would be disposed on the left and right clavicle. It is contemplated that the left and right therapy providing devices may be programmed or be configured to delivery different therapies. For example the left and right therapy providing devices may be configured to deliver different frequency ranges of therapy chosen from the ranges disclosed above. Additionally, the devices may be activated, programmed or configured to deliver therapy out of phase with each other. For example, the left therapy providing device may include a delay in the therapy, such that the delay in the therapy may cause a standing wave to be generated between the two devices.

It is further contemplated that a single therapy providing device may be disposed on the sternum to provide therapy as provided herein. Further still, two therapy providing devices maybe disposed on the sternum in order to provide the therapy as contemplated herein.

In accordance with the present disclosure, according to some embodiments, the therapy device may be factory programmed to utilize a certain frequency or range of frequencies to provide therapy. Alternatively, the frequencies may be selected and programmed or chosen from memory by a health care provider based upon a patient's response to a specific frequency or range of frequencies.

It is further contemplated that the computing device may be additionally in communication with other sensors, such as a blood pressure monitor, heart rate monitor, pulse oximetry monitor, electrocardiogram (EKG/ECG), or glucose sensor.

In one embodiment the computing device 800 would receive data from the blood pressure monitor, or other sensor, such that the user's blood pressure would be recorded before, during and after the application of therapy in accordance with the present disclosure, according to some embodiments. This data, along with the therapy data could be provided to the user and/or a health care provider. Based upon the data, the frequency or range of frequencies selected for therapy could be adjusted. The adjustments may be made automatically by the program, or by a health care provider or by the user themselves.

In another embodiment, the processor of the therapy providing device 200 may be in communication with other sensors, such as those described above, wherein the other sensors would be coupled in communication with the therapy providing device. The processor within the therapy providing device 200 can receive data from various other sensors, such as a blood pressure monitor. The data received from the blood pressure monitor may be utilized by the program within the memory of the electronics module to further control the therapy providing device 200.

The signals generated by the program and transmitted to the therapy providing device are preferably in the form of a sine wave. However, other wave forms may be utilized, such as a square waveform, sawtooth waveform or triangle waveform.

It is further contemplated that additional sensors maybe utilized with the methods and devices in accordance with the present disclosure, according to some embodiments. For example, a blood pressure monitor may be affixed to the patient as described above. Other sensors, such as a sleep sensor, movement sensor, pulse oximetry sensor, temperature sensor, heart rate monitor, EKG, microphone, digital stethoscope, light sensor, sleep apnea device (CPAP) or camera may be used in combination with the therapy system 100 in accordance with the present disclosure, according to some embodiments. The sensors listed above could be used separately or in combination to provide additional data to the user or a health care provider as to the health of the user as well as to the response of the user to the therapy provided by the therapy system 100.

It is further contemplated that any of the above sensors could be incorporated into the therapy providing device 200 in accordance with the present disclosure, according to some embodiments. If incorporated into the therapy providing device 200, the data from each of the additional sensors could be utilized by the program to alter the therapy provided based upon data received from the various sensors. In an alternative embodiment, the data from each of the additional sensors could be stored on the resident memory contained within the therapy providing device 200. The therapy providing device 200 could then be turned into a service center after a period of time, wherein the data contained within the memory can be retrieved and analyzed. In yet another embodiment, the data stored within the memory can be downloaded from the therapy providing device 200 each time the therapy providing device is placed on the inductive charging pad. The data can then be transmitted to a collection center and analyzed. Additionally, the data could be uploaded to a server or other internet/network connected personal computer, such that the data could be viewed by the user, a health care provider or others.

In another embodiment, the device will store the number of uses and durations of usage to allow the health care practitioner to determine compliance of the patient. As in sleep apnea devices, reimbursement is only allowed if the patient is compliant in using the device for a certain percentage of use by tracking and recording the usage of the therapy providing device of the present disclosure, according to some embodiments, this data could be utilized for reimbursement purposes.

In another embodiment, the therapy providing system 100 of the present disclosure, according to some embodiments could be associated with a home health system, such as Honeywell's HOMMED system. In this embodiment, the therapy providing system 100 in accordance with the present disclosure, according to some embodiments would be coupled to a monitoring system. In this embodiment, a health care provider could remotely monitor users as well as their response to the therapy being provided. Further still, the therapy providing system 100 may be configured to recognize an emergency, such as excessively high blood pressure, excessively low blood pressure, high heart rate or low heart rate and generate an alert, such as an alarm or notification to an emergency response unit to request help for the user.

In accordance with the present disclosure, according to some embodiments, the therapy providing device 200 as described herein is disposed adjacent to or thereabout the clavicle just above the brachial plexus of the user. It is contemplated that the therapy providing device 200 may be placed at other locations on the user such as the sternum, jaw, scapula, kneecap, wrist or skull. When activated, the driver assembly 220 of the therapy providing device 200 generates a frequency in the form of a sound wave; this sound wave is transmitted to the clavicle and the skin adjacent the clavicle. The sound waves transmitted to the clavicle are transmitted in the form of vibrations. The vibrations travel through the clavicle and into the skin, the arteries, vessels, nerves, sensory corpuscles, airways, bones near the clavicle, ligaments and tendons. As a result, the vibrations are eventually transmitted to the baroreceptors, the nociceptors, the proprioreceptors and other somatosensory sensors. Here, the vibrations interact with the baroreceptors and other sensors in a manner to lower blood pressure. In a preferred embodiment, the clavicle is chosen because it's easily accessible location as well as its ability to transmit sound or vibrations. The clavicle is easy to identify by a health care provider and a patient as it resides close to the surface of the skin regardless of body mass.

In accordance with the methods and devices of the present disclosure, according to some embodiments, activation of both, the carotid and aortic baroreceptors as well as other somatosensory sensors can be achieved. It is believed that activation of both the carotid and aortic baroreceptors is beneficial in achieving lower blood pressure. It is believed that the methods provided according to the present disclosure, according to some embodiments mimic exercise, and therefore achieve a lowering of blood pressure. In accordance with the present disclosure, according to some embodiments, the vibration provided by the therapy providing devices 200 activates a baroresponse resulting in a lowering of blood pressure.

In accordance with the disclosure, the therapy may be provided at night time either right before the patient enters a sleep cycle or during a sleep cycle of the patient. It may be beneficial to provide the therapy in accordance with the present disclosure, according to some embodiments at night time as, not to be limited by theory, it is believed that in some cases, one of the most important times to lower blood pressure is during the night. By providing therapy at night time in accordance with some embodiments of the present disclosure, the therapy can be utilized to address nighttime hypertension. Additionally, at night time, systemic drug levels can be at their lowest, therefore there is a need for additional blood pressure control at this time. The therapy providing device of some embodiments of the present disclosure may be configured to provide two or more therapy providing sessions during a night time. As such, the therapy session after the first therapy session may be configured to initially begin at a lower volume/vibration or extension of the vibration source. By beginning the second or subsequent therapy sessions at a lower setting, the intention is to not wake the user from their sleep, over a period of pre-determined time, the volume/vibration or extension of the vibration source may increase.

In accordance with another embodiment of the present disclosure, it is believed that through the use of a single therapy providing device instead of two therapy providing devices can be utilized to lower only Diastolic blood pressure, wherein the use of both therapy providing devices can be utilized to lower both Systolic and Diastolic blood pressure. In some embodiments, systems and methods as described herein could lower systolic, diastolic, and/or mean arterial blood pressure by about or at least about 5 mm Hg, 10 mm Hg, 15 mm Hg, 20 mm Hg, 25 mm Hg, 30 mm Hg, 35 mm Hg, 40 mm Hg, 45 mm Hg, 40 mm Hg, or more with respect to pre-treatment blood pressures. In some embodiments, systems and methods as described herein could lower systolic, diastolic, and/or mean arterial blood pressure by about or at least about 3%, 4%, 5%, 6%, 7%, 8%, 9%, 10%, 15%, 20%, 25%, 30%, 35%, 40%, 45%, 50%, or more with respect to pre-treatment blood pressures.

It is contemplated that the therapy providing devices of some embodiments of the present invention may be utilized to raise blood pressure, ease snoring, and/or cause a relaxing sensation. If it is desired that the therapy system 100 of the present disclosure, according to some embodiments is utilized to ease snoring, the therapy providing device can include a program, where the program utilizes sensors, such as: a motion sensor, an accelerometer sensor or using a microphone of the therapy providing device 200 as previously described to detect snoring of the user. The program can include an algorithm to distinguish a user's snoring sounds from ambient sounds. Once the program determines that the user is snoring, the therapy providing device 200 would be activated to deliver acoustic vibrations to the user to cause the user to wake or change sleeping position, thereby alleviating the snoring. In this embodiment, if the therapy providing system 100 is only being utilized to address a user's snoring, then only one therapy providing device 200 is needed to be used, wherein the single therapy providing device 200 may be disposed on the user at locations different than those described herein, for example the single therapy providing device may be disposed on the user's chest/sternum, wrist, skull, jawbone, hand, back of the neck or forehead. However, if the therapy providing device is delivering both hypertensive therapy as well as addressing a user's snoring then two therapy providing devices 200 can be utilized as described herein, wherein the therapy system 100 can be configured to run both the therapy program and the snoring program simultaneously or the two programs can be combined into a single program.

In accordance with the disclosure, the therapy may be provided prior to a user's sleep cycle and again in the morning either before they awake or shortly after they have woken up.

In accordance with the disclosure, the therapy providing device may be programmed with frequencies, wherein other frequencies may be utilized to raise blood pressure at such times whereby raising the blood pressure would be therapeutic and beneficial to a patient. It may be desirable to raise blood pressure after childbirth or to counteract episodes of hypotension.

It is further contemplated that the device and methods according to the present disclosure, according to some embodiments may be utilized at any time. For example, it may be desirable to utilize the device during the day time, where the device could be utilized in combination with a blood pressure monitor, or alternatively, incorporate a blood pressure monitor for closed loop control. In this embodiment, the program would monitor the user's blood pressure and apply therapy on an as needed basis. The user could select to turn the system off if desired, for example if they are planning to engage in physical activity which will raise their blood pressure.

It is further contemplated that the therapy providing device of the present disclosure, according to some embodiments may be utilized to train or re-train a user's body to respond to its own nerve signals. It is contemplated that the therapy providing device of the present disclosure, according to some embodiments may only need to be utilized for a period of time, and such, over time the user's body would regain control over the user's blood pressure. It is contemplated that the therapy providing device of the present disclosure, according to some embodiments may only be needed to be utilized for a period of time such as, 1 day, 2 days, 3 days, 4 days, 5 days, 6 days, 7 days, 8 days, 9 days, 10 days, 11 days, 12 days, 13 days, 14 days, 15 days, 16 days, 17 days, 18 days, 19 days, 20 days, 21 days, 22 days, 23 days, 24 days, 25 days, 26 days, 27 days, 28 days, 29 days, 30 days, 31 days, 2 months, 3 months, 4 months, 5 months, 6 months, 7 months, 8 months, 9 months, 10 months, 11 months, 1 year or more. It is contemplated that the user may reduce the amount of time they utilize the therapy providing device over time. For example, the user may be required to utilize the therapy providing device on a daily basis initially, and over time, they may begin to need to utilize the therapy providing device less as their bodies own system becomes trained or re-trained to control the user's blood pressure.

### Test Results

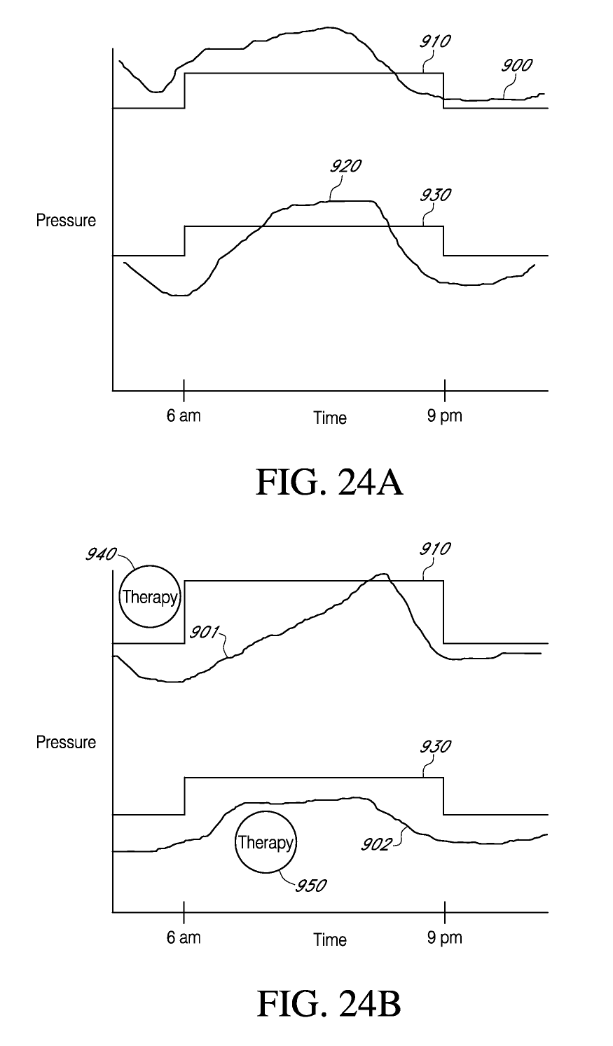

In accordance with the present disclosure, according to some embodiments, and referring to FIGS. 24A and 24B, the following blood pressure results were achieved through use of the device and methods described herein. FIGS. 24A and 24B illustrate ambulatory blood pressure readings over a 24 hour period. Line 910 is the European Society of Hypertension (ESH), the UK National Institute for Health and Clinical Excellence (NICE) and American Society of Hypertension (ASH) recommended limits for Systolic blood pressure. Between the hours of 10 pm and 7 am a blood pressure of below 125 mmHg is considered to be at goal. During the daytime between the hours of 7 am and 10 pm, a blood pressure below 140 mmHg is considered to be at goal. Line 930 is the ESH, NICE and ASH recommended limits for Diastolic blood pressure, similar to the Systolic line 910, between the hours of 10 pm and 7 am an at-goal Diastolic pressure is considered to be 80 mmHg, and between the hours of 7 am to 10 pm a measurement of 90 mmHg is considered to be at goal.

As shown in FIG. 24A, lines 900 and 920 represent twenty-four (24) hour ambulatory blood pressure measurements of an individual, wherein blood pressure measurements were taken every fifteen (15) minutes. The user presented in FIG. 24B would be considered to be hypertensive—that is to have high blood pressure. This can be determined by looking specifically at lines 900 and lines 920, wherein any time these lines are above the recommend guideline pressures, lines 910 and 930 the user would be considered to be hypertensive.

Referring now to FIG. 24A there is shown a graph of the same user after having received therapy in accordance with the present disclosure, according to some embodiments. In this instance, the user received therapy at twice for two hours (2 hours) each time as depicted items 940 and 950. Comparing the user's actual blood pressure measurements, lines 900 and 920 of FIG. 24A, with the user's treated actual blood pressure measurements, lines 901 and 902 of FIG. 24A, it can be clearly seen that the user's blood pressure was significantly lowered through the application of therapy utilized the device and methods of the present disclosure, according to some embodiments.

To achieve the results depicted in FIG. 24A, two therapy providing devices were utilized, one on the left clavicle and one on the right clavicle. A frequency between 60 and 100 Hz was delivered by the speaker of each therapy providing device. The therapy providing devices were utilized for a total of 4 hours of therapy, wherein the frequency of 65 Hz was played for 8 seconds, followed by a sweep of frequencies from 65 Hz to 98 Hz lasting 1 second, afterwards 98 Hz was played for 8 seconds, followed by a sweep of frequencies from 98 Hz to 65 Hz lasting for 1 second. Therapy was provided, repeatedly for 120 minutes following this cycle. After 120 minutes the therapy was suspended for a period of 4 hours. After 4 hours of silence, an additional 120 minutes of therapy was delivered utilizing the cycle above.

Blood pressure measurements were taken before the application of the therapy, whereby the user's Systolic blood pressure averaged 131 mmHg at night time and 144 mmHg during the day. Diastolic blood pressure was 72 mmHg at night time and 87 mmHg during the day. After using the therapy for one evening (one 8 hour session as described above), Systolic blood pressure averaged 116 mmHg at nighttime and 131 mmHg at daytime and diastolic blood pressure averaged 66 mmHg at nighttime and 80 mmHg at daytime.

### Method of Action

In accordance with the present disclosure, according to some embodiments, as described in detail above and with reference to the included publications, and not to be limited by theory, it is understood that baroreceptors and nerves affect blood pressure through a measured response generated by stretching or contraction of the arterial wall.

Nerve fibers, including baroreceptors, have the following input-output characteristics; threshold pressure, saturation, post-excitatory depression (PED), Asymmetric Rate Sensitivity and hysteresis.

As long as pressure within an artery remains below a certain level, no nerve firing occurs, this is referred to as the nerve threshold pressure. Above the threshold pressure, the fiber responds by producing action potentials, i.e. as signal. Individual fibers within humans and animals possess a wide range of pressure threshold values.

As pressure increases within the artery, the firing rate of individual fibers increases. However, at certain pressure, further increases in input yield no further increase in output frequency, thereby reaching the saturation of the baroreceptor nerve.

If pressure input within the artery is stepped from a low pressure, which is higher than the threshold pressure, to a higher pressure, then returning to a lower pressure level, will result in a brief period of shutoff, that is there will be no firing of the baroreceptor nerve, also referred to as post-excitatory depression (PED). The baroreceptor nerve will return to its original firing rate after time.

Baroreceptor nerve frequency response to rising pressure is more pronounced than the response to falling pressure, otherwise known as asymmetric rate sensitivity.

Lastly, periodic inputs produce looping in pressure-frequency plots, another indication of the asymmetry between responses to rising and falling pressures otherwise referred to as hysteresis.

In accordance with the present disclosure, according to some embodiments, utilization of the devices in accordance with the methods described herein cause an activation of the nervous system which affect blood pressure. The nerve terminal endings respond to stretch or acoustic vibration, and produce a frequency-modulated train of action potentials which can override the natural frequencies to elicit a response. Wherein the therapy provided by the disclosure, utilizes acoustic vibration of specific frequencies applied at specific time intervals to activate the body's nervous system to elicit a blood pressure response. The therapy of the present disclosure, according to some embodiments is applied in a cyclic manner as it is believed that the baroreceptors may become saturated if stimulated for too long of a period of time. If the therapy was applied continuously it is believed that the baroreceptors would stop responding.

According to a method of the present disclosure, according to some embodiments, the therapy providing device is disposed adjacent to a user's clavicle. The clavicle, being a dermal bone, is capable of transmitting vibrations. The clavicle lies above the cervicoaxillary which holds auxiliary arteries, veins, airways and the brachial plexus of nerves that supply the upper limb of the arm. Vibrating the clavicle is believed to create micro-pulsations which travel to the Aortic Baroreceptors and the Carotid Bulb Baroreceptors. These micro-pulsations are believed to be perceived as an increase in heart rate by the baroreceptors which then send a signal to the brain, thereby causing the body to lower blood pressure. It is further contemplated that the acoustic vibrational energy provided by the therapy device(s) of the present disclosure, according to some embodiments may stimulate the vagus nerve thereby causing a lowering in blood pressure. It is additionally believed that the acoustic vibrational energy of the therapy device(s) of the present disclosure, according to some embodiments is transmitted to the user's lungs, where the vibrational energy activates and/or inhibits the sympathetic and/or parasympathetic nervous system.

Selective stimulation of primary nerve endings can be obtained with careful control of the amplitude, displacement and the mode of application of the vibration or micro-pulsations.

It is believed that the therapy providing device in accordance with the present disclosure, according to some embodiments when disposed on a user's clavicle, thorax, jaw, patella and activated to produce a vibration or a plurality of vibrations using a transition, sweeping or stepping pattern for a period of time is believed to cause a baroresponse thereby causing an effect on blood pressure.

It is also contemplated in accordance with some embodiments of the present invention that the therapy providing device may be utilized in accordance with the disclosure herein to affect a user's sleep quality, such as the reduction or cessation of snoring or sleep apnea. Further still the therapy providing device may further benefit users who suffer from migraine headaches.

It is contemplated that various combinations or subcombinations of the specific features and aspects of the embodiments disclosed above may be made and still fall within one or more of the inventions. Further, the disclosure herein of any particular feature, aspect, method, property, characteristic, quality, attribute, element, or the like in connection with an embodiment can be used in all other embodiments set forth herein. Accordingly, it should be understood that various features and aspects of the disclosed embodiments can be combined with or substituted for one another in order to form varying modes of the disclosed inventions. Thus, it is intended that the scope of the present inventions herein disclosed should not be limited by the particular disclosed embodiments described above. Moreover, while the invention is susceptible to various modifications, and alternative forms, specific examples thereof have been shown in the drawings and are herein described in detail. It should be understood, however, that the invention is not to be limited to the particular forms or methods disclosed, but to the contrary, the invention is to cover all modifications, equivalents, and alternatives falling within the spirit and scope of the various embodiments described and the appended claims. Any methods disclosed herein need not be performed in the order recited. The methods disclosed herein include certain actions taken by a practitioner; however, they can also include any third-party instruction of those actions, either expressly or by implication. For example, actions such as “positioning a device for producing vibrational energy over a bony structure” include “instructing the positioning of a device for producing vibrational energy over a bony structure.” The ranges disclosed herein also encompass any and all overlap, sub-ranges, and combinations thereof. Language such as “up to,” “at least,” “greater than,” “less than,” “between,” and the like includes the number recited. Numbers preceded by a term such as “approximately”, “about”, and “substantially” as used herein include the recited numbers, and also represent an amount close to the stated amount that still performs a desired function or achieves a desired result. For example, the terms “approximately”, “about”, and “substantially” may refer to an amount that is within less than 10% of, within less than 5% of, within less than 1% of, within less than 0.1% of, and within less than 0.01% of the stated amount.

What is claimed is:

1. A device for modulating blood pressure, comprising:
a vibration source configured to apply vibratory energy to the clavicle through the skin surface; and
a bandage comprising at least a first layer and a second layer, at least one of the layers having an aperture formed therethrough sized to receive a portion of the vibration source, the bandage being adapted to adhesively attach to a skin surface over a clavicle of a subject, wherein at least one of the layers comprises at least one biologically active compound, wherein the biologically active compound is spaced apart from the vibration source forming a biologically compound-free area on the skin when the bandage is attached to the skin surface.

2. The device of claim 1, wherein the biologically active compound comprises an anti-hypertensive agent.

3. The device of claim 2, wherein the anti-hypertensive agent is configured to be absorbed transdermally.

4. The device of claim 3, wherein the anti-hypertensive agent comprises nitropaste.

5. The device of claim 3, wherein the anti-hypertensive agent comprises clonidine.

6. The device of claim 1, wherein the vibration source is configured to apply vibratory energy at a frequency of between about 35 Hz and about 150 Hz.

7. The device of claim 1, wherein at least one of the first layer and the second layer of the bandage comprises an adhesive.

8. The device of claim 1, wherein the adhesive forms a discontinuous layer.

9. The device of claim 8, wherein the discontinuous layer comprises a plurality of spaced-apart strips.

10. The device of claim 8, wherein the discontinuous layer comprises a plurality of spaced-apart dots.

11. A device for modulating blood pressure, comprising:
a vibration source configured to apply vibratory energy to the clavicle through the skin surface; and
a bandage comprising at least a first layer and a second layer, at least one of the layers having an aperture formed therethrough sized to receive a portion of the vibration source, the bandage being adapted to adhesively attach to a skin surface over a clavicle of a subject, wherein at least one of the first layer and the second layer of the bandage comprises an adhesive forming a discontinuous layer comprising an adhesive-free zone.

12. The device of claim 11, wherein the discontinuous layer comprises a plurality of spaced-apart strips.

13. The device of claim 11, wherein the discontinuous layer comprises a plurality of spaced-apart dots.

14. A method for modulating blood pressure, comprising:
positioning a device comprising a vibration source and at least one biologically-active compound on the skin surface of a patient;
activating the vibration source such that the vibration source modulates the patient's baroreceptors, thereby decreasing the blood pressure of the patient; and
absorbing the biologically-active compound transdermally through the skin surface of the patient.

15. The method of claim 14, wherein the biologically active compound comprises an anti-hypertensive agent, and wherein absorbing the anti-hypertensive agent decreases the blood pressure of the patient.

16. The method of claim 14, wherein the skin surface of a patient is proximate a clavicle of the patient.
